(19) United States
(12) Patent Application Publication
(10) Pub. No.: US 2025/0059496 A1
(43) Pub. Date: Feb. 20, 2025

(54) PRODUCTION OF ALCOHOLS AND THEIR PRECURSORS BY GENETICALLY MODIFIED ETHANOLOGENIC BACTERIA

(71) Applicant: Chemical Evolution Ltd., Calgary (CA)

(72) Inventors: Dustin LILLICO, Calgary (CA); Trevor RANDALL, Calgary (CA); Alejandra ENRIQUEZ, Calgary (CA); Markus WEISSENBERGER, Calgary (CA)

(21) Appl. No.: 18/781,788

(22) Filed: Jul. 23, 2024

(30) Foreign Application Priority Data
Aug. 4, 2023 (CA) 3208555

(51) Int. Cl. C12N 1/20; C12N 9/42; C12N 15/74; C12P 7/06

(52) U.S. Cl. CPC C12N 1/20; C12N 9/2437; C12N 9/2445; C12N 15/74; C12Y 302/01004; C12Y 302/01021; C12P 7/065

(57) ABSTRACT

A genetically modified ethanologenic organism which comprises:
a. an exoglucanase (cex-like) polynucleotide sequence with at least 70% sequence coverage to SEQ 1 or SEQ 68, and at least 70% sequence identity to SEQ 1 or SEQ 68; and
b. a β-glucosidase 1 (bg11) polynucleotide sequence with at least 70% sequence coverage to SEQ 3 or SEQ 14, and at least 70% sequence identity to SEQ 3 or SEQ 14.

### REFERENCE TO AN ELECTRONIC SEQUENCE LISTING

The contents of the electronic sequence listing (20241106_SequenceListing_ST26_23156197US1.xml; Size: 128,189 bytes; and Date of Creation: Nov. 6, 2024) is herein incorporated by reference in its entirety.

### FIELD OF THE INVENTION

The present invention relates to a method to produce alcohols and alcohol precursors through genetically manipulated ethanologenic bacteria that degrades cellulose and ferments the remaining sugar source.

### BACKGROUND OF THE INVENTION

Biofuel is increasingly becoming a necessity in order to wean off the human consumption of fossil fuels in aspects of everyday life, transport and home heating being the largest two industries of focus. As an alternative energy source to oil and coal, the main feedstock for the production of bioethanol and other bioalcohols is starch which can yield its sugar much more readily than cellulose. This is due to the difference in structure as starch contains glucose molecules connected through α-1,4 linkages and cellulose comprises of glucose molecules attached through β-1,4 linkages. The β-1,4 linkages allow for crystallization of the cellulose, leading to a more rigid structure, which is more difficult to break down using physical, chemical, and biological processes.

The limitation that comes from solely concentrating the production of bioethanol on extracting the sugars from starches is that it prevents the utilization of the larger portion of biomass which comes in the form of lignocellulosic biomass (contains lignin, cellulose, and hemicellulose) present in almost every plant on earth. A delignification reaction allows the recovery of cellulose from those lignocellulosic plants. Further degradation of the cellulose generates cellobiose and/or glucose which can be utilized for the production of ethanol using various biology-based processes.

Seen as a sustainable alternative to gasoline and with the goal of alleviating many countries' dependence on foreign oil, the bioethanol industry is still hampered by its dependence on corn or sugar cane as main sources of fuel, as they are both rich in starch. It is estimated that about 45% of all corn production in the U.S. is directed to ethanol fuel production. This is a situation which has disastrous consequences when the prices of gasoline decline significantly low making corn-based bioethanol unsustainable on a price viewpoint.

Across the world, many other large ethanol-producing countries, including China and Brazil, have shown some struggles in ethanol production from biomass as many companies are carrying large debts from the implementation of such processes in conjunction with large processing plants having to be shut down or decrease production.

In Asia, palm oil prices have recently increased to their highest levels in years, which, in turn, will hamper the ability of Indonesia and Malaysia to produce local biofuel. Oil palm trunk is a valuable and plentiful resource in those countries to generate biofuels and biochemicals. Oil palm trunk contains a large amount of starch which is more readily solubilized in water, compared to cellulose. The starch can then be heated and hydrolyzed to glucose by amylolytic enzymes without pre-treatment. However, the conventional Oil palm trunk treatment requires high capital and operational costs and is therefore prohibitive to market entry. Moreover, the treatment carries a high probability of microbial contamination during starch processing which significantly reduces purity and poses additional complications in refining procedures.

In Europe, the biofuel industry (including biodiesel and bioethanol production) depends heavily on food-based feedstocks like vegetable oils (i.e., rapeseed, palm oil, soy) for biodiesel and corn, wheat, and sugar beet for bioethanol. Simultaneously, concerns have been raised that making fuel out of crops displaces other crops and can inflate food prices. These concerns are leading to policy changes that incentivize a shift away from food-based biofuels.

The pivot from starches to cellulose for the production of glucose is preferable as it will cease to use a food source to generate glucose. However, the costs to do so are currently prohibitive. Cellulosic ethanol as it is called relies on the non-food component of a plant to be used to generate ethanol. This would allow the replacement of the current more widespread approach of making bioethanol by using corn or sugarcane. The diversity and abundance of these types of cellulose-rich plants would allow for food resources to remain largely intact and capitalize on the waste generated from these food resources (such as cornstalk and stover) to generate ethanol. Other cellulose sources such as straws, algae and even trees fall under cellulose-rich biomass sources which can be used in generating ethanol if a commercially viable process is developed.

The reason why starches are preferred to cellulose-rich sources to generate ethanol is that the extraction of glucose from cellulose is substantially more difficult and resource intensive. To better understand the conditions associated with this increased difficulty, it is worthwhile describing the structural similarities and differences between starch and cellulose.

Cellulose and starch are polymers which have the same repeat units of glucose. However, the differences between starch and cellulose can be seen in the way the repeating glucose monomers are connected to one another. In starch, the glucose monomers are oriented in the same direction. In cellulose, each successive glucose monomer is rotated 180 degrees in respect to the previous glucose monomer. This, in turn, ensures that the bonds between each monomeric glucose differs between starch and cellulose. In starch, the bonds (otherwise known as links between the monomers) are referred to as α-1,4 linkages, in cellulose these bonds are referred to as β-1,4 linkages (see FIG. 1).

The difference between these bonds impacts the chemical characteristics of starch and cellulose. Starch can dissolve in warm water while cellulose does not. Starch can be digested by humans, cellulose cannot. In general, starch is structurally weaker than cellulose partly due to its geometrical make-up which is less crystalline than cellulose. Starch is, at its core, a method for plants to store energy due to the reversibility of the α-1,4 linkage, therefore extracting sugars from starch is much easier than to do so from cellulose as the latter's core function is to provide structural support.

As the main component of lignocellulosic biomass, cellulose is a biopolymer consisting of many glucose units connected through β-1,4-glycosidic bonds (see FIG. 1). Glucose has two isomers: α-glucose (present in starches as branched polymers) and β-glucose (present in cellulose connected via a β-1,4-glycosidic bond with one β-glucose monomer rotated by 180 degrees relative to its neighbour). A cellulose molecule can comprise between hundreds to thousands of glucose units. Since the cellulose molecules are linear, due in part to intermolecular hydrogen bonding, neighboring cellulose molecules can be very closely packed, and, in turn, provide the structural strength necessary to support plants.

### Hydrolysis of Cellulose

The hydrolysis of cellulose is the rate limiting step in the conversion of cellulose into biofuel. The processes currently using cellulose as a starting material for bioethanol production require the conversion of cellulose into cellobiose, further processing to generate glucose, and the final generation of ethanol. The fermentation of glucose using ethanologenic organisms is what leads to the production of ethanol. While that last step in biofuel production has been mastered for some time, the rate limiting step of cellulose hydrolysis is the most crucial one which hinders a wider acceptance of biofuels. The difficulty in overcoming this conversion of cellulose into glucose lies with the fact that cellulose has a crystalline structure which renders its conversion to glucose quite difficult because of the close packing of multiple cellulose polymers. This close packing imparts cellulose its inherent stability under a variety of chemical conditions. For example, cellulose polymers are generally insoluble in water, as well as a number of organic solvents. Furthermore, cellulose is also generally insoluble when exposed to weak acids or bases.

In general, there are three main approaches to hydrolyze cellulose: mechanical or physical, chemical and enzymatic. The chemical method resorts to the use of concentrated strong acids to hydrolyze cellulose under conditions of high temperature and pressure. Many different types of acids, such as HCl and H₂SO₄, have been used in the past to achieve this. The use of one of these acids usually results in at least one of the following drawbacks: corrosion of the reaction vessel, difficulty of disposing of the discharged reactants, and others. The biofuel industry is generally reticent to use chemically hydrolyzed cellulose because of the presence of toxic by-products in the resulting glucose. These by-products, if introduced in the fermentation step, will negatively affect the delicate balance of the fermenting yeast.

### Cost of Enzymatic Hydrolysis

It is known that the costs to extract biofuel from cellulose are higher than when doing so from starch. It is estimated that, on average, depending on location and availability of biomass, the cost for cellulose conversion is about 50% more that starch conversion to glucose. This means that there currently is a clear barrier to producers for using cellulose rather than corn or other starch resources to generate glucose from biomass.

It is generally understood that roughly half of the total cost of producing biofuel from cellulose stems from the price of the enzymes (cellulases and hemicellulases). The generation of enzymes for enzymatic hydrolysis of cellulose is a time-consuming process and large volumes of enzyme are required to render the process commercially viable. One possible approach is to improve the rate of the hydrolysis reaction which, in turn, would result in a decrease in the overall cost of the process.

The enzymatic approach to hydrolyzing cellulose uses enzymes to carry out the hydrolysis reaction. Enzymes, such as cellulases (comprising endoglucanases; exoglucanases; and β-glucosidases) are used for the conversion of cellulose into glucose, however each requires extensive controls in place to maximize the reaction rates the enzymatic approach is expected to provide. Conditions such as temperature, pH, salinity, concentration of substrate and product are all factors that may affect enzyme activity with even small deviations of these parameters from the enzyme's optimal conditions resulting in loss of function. Overall, with many controls needing to be taken into consideration with the enzymatic hydrolysis of cellulose along with strict enzyme-specific functional conditions, such a method can render the process cost prohibitive in some cases and/or limit their implementation.

The enzymatic hydrolysis of cellulose is, as seen from the above, limited by the structure of cellulose itself but also by the approaches taken to degrade it into a biofuel. The production of a robust, low-cost process from cellulose has not yet been achieved. In this sense, genetically modified organisms have been employed for part of the entirety of the cellulose to ethanol pathway.

U.S. Pat. No. 4,496,656A describes a process for production of cellulase according to the present invention thus comprises culturing a cellulase-producing microorganism belonging to Cellulomonas uda CB4 in a cellulose-containing medium and recovering the cellulase produced from the culture broth. According to the present invention, because the bacteria belonging to Cellulomonas uda CB4 is capable of producing a cellulase having a high activity, not found in the reports of the prior art, in a culture medium, it is possible to produce a cellulase having a high crystalline cellulose decomposing activity comparable to those produced from a mold within a short cultivation period of two days.

In the paper titled “Expression of a cellulase gene in Zymomonas mobilis” by Misawa et al. (J. Biotechnology, 1988, 7 (3), 167-177), it was reported that a cellulase (CMCase) gene of Cellulomonas uda CB4 was introduced into Zymomonas mobilis NRRL B-14023 on pZA22, a cloning vector for Zymomonas, by conjugal transfer. Z. mobilis carrying this gene synthesized cellulase immunologically identical with that of C. uda CB4, by transcriptional read-through from the promotor of the chloramphenicol resistance (chloramphenicol acetyltransferase) gene within pZA22. A strong promotor containing a translation initiation signal was obtained from Z. mobilis NRRL B-14023 chromosomal DNA. By gene fusion between this Zymomonas promotor fragment and the truncated cellulase gene, the activity of cellulase synthesized in Z. mobilis reached 0.78 units per ml culture, six-fold higher than that by transcriptional read-through from the promotor of the chloramphenicol resistance gene.

In the paper titled “Expression of an endoglucanase gene of Pseudomonas fluorescens var. cellulosa in Zymomonas mobilis” by Lejeune et al. (FEMS Microbiology Letters, 1988, 49 (3), 363-366), the authors reported that by using a broad host-range, mobilizable plasmid vector, the endoglucanase gene eglX from Pseudomonas fluorescens var. cellulosa was cloned and expressed in the bacterial ethanologen Zymomonas mobilis. The enzyme was intracellular in this new host. It was produced throughout the growth phase and was not repressed by glucose. We postulate that transcription of the eglXgene was initiated from a promoter of the vector in Z. mobilis.

In the paper titled “Heterologous Expression and Extracellular Secretion of Cellulolytic Enzymes by Zymomonas mobilis” by Linger et al. (Appl Environ Microbiol. 2010 October; 76(19): 6360-6369), the authors reported that by using a technique known as consolidated bioprocessing (CBP), the initial steps toward achieving a single organism to convert pretreated lignocellulosic biomass to ethanol in the fermentation host Zymomonas mobilis were investigated. This was achieved by expressing heterologous cellulases and subsequently examining the potential to secrete these cellulases extracellularly. Numerous strains of Z. mobilis were found to possess endogenous extracellular activities against carboxymethyl cellulose, suggesting that this microorganism may harbor a favorable environment for the production of additional cellulolytic enzymes. The heterologous expression of two cellulolytic enzymes, E1 and GH12 from Acidothermus cellulolyticus, was examined. Both proteins were successfully expressed as soluble, active enzymes in Z. mobilis although to different levels. While the E1 enzyme was less abundantly expressed, the GH12 enzyme comprised as much as 4.6% of the total cell protein. Additionally, fusing predicted secretion signals native to Z. mobilis to the N termini of E1 and GH12 was found to direct the extracellular secretion of significant levels of active E1 and GH12 enzymes. The subcellular localization of the intracellular pools of cellulases revealed that a significant portion of both the E1 and GH12 secretion constructs resided in the periplasmic space. The results obtained strongly suggest that Z. mobilis can support the expression and secretion of high levels of cellulases relevant to biofuel production, thereby serving as a foundation for developing Z. mobilis into a CBP platform organism.

In the paper titled “Evaluation of Cellulase Production by Zymomonas mobilis” by Todhanakasem and Jittjang (Bioresources, 2017, 12(1), 1165-1178), the authors report the use of Z. mobilis as a potential microbe for consolidated bioprocessing to convert lignocellulosic biomass to fermentable sugars while at the same time producing ethanol. To achieve this goal, Z. mobilis must be evaluated for the production of cellulolytic enzyme. This work reports on the potential of intracellular and extracellular crude extracts from Z. mobilis ZM4 and TISTR 551 to hydrolyze various cellulosic materials including carboxymethylcellulose (CMC), delignified rice bran, microcrystalline cellulose, and filter paper. Crude intracellular extracts from ZM4 and TISTR 551 showed high endoglucanase activity with CMC substrates at an optimal pH of 6 to 7 and temperature range of 30 to 40° C. The endoglucanase activity from the crude extracts was significantly higher than the exoglucanase activity. Of the high crystalline celluloses substrates tested, the best results were obtained for the hydrolysis of delignified rice bran by crude intracellular enzyme extracts of Z. mobilis TISTR 551.

In the paper by Vasan et al. titled “Cellulosic ethanol production by Zymomonas mobilis harboring an endoglucanase gene from Enterobacter cloacae” (Bioresource Technol. 2011, 102(3), 2585), the authors report the use of a 2.25-kb fragment conferring cellulase activity from Enterobacter cloacae (isolated from the gut of the wood feeding termite, Heterotermes indicola), cloned in Escherichia coli. The cloned fragment contained a 1083-bp ORF which could encode a protein belonging to glycosyl hydrolase family 8. The cellulase gene was introduced into Zymomonas mobilis strain Microbial Type Culture Collection centre (MTCC) on a plasmid and 0.134 filter paper activity unit (FPU)/ml units of cellulase activity was observed with the recombinant bacterium. Using carboxymethyl cellulose and 4% NaOH pretreated bagasse as substrates, the recombinant strain produced 5.5% and 4% (v/v) ethanol respectively, which was threefold higher than the amount obtained with the original E. cloacae isolate. The recombinant Z. mobilis strain could be improved further by simultaneous expression of cellulase cocktails before utilizing it for industrial level ethanol production.

In light of the above, it is clear that there is an unmet need for a process to generate alcohol and alcohol precursors from a lignocellulosic biomass material that is not reliant on enzymatic cocktails due to their cost. In that respect, the use of a single organism that contains cellulolytic as well as fermentation capabilities is much more highly attractive as it will potentially lower the costs of bioethanol production.

### SUMMARY OF THE INVENTION

The embodiments of the present invention are related to novel organisms for the production of alcohols and alcohol precursors using cellulose-based materials. More particularly, the invention encompasses methods for the genetic modifications of an ethanologenic organism which allows the organism to produce alcohols and alcohol precursors in association with cellulose degradation through the introduction of genes isolated from prokaryotic and/or eukaryotic organisms. These genes associated with cellulose degradation are then integrated or introduced into another prokaryotic organism. Through the inclusion of cellulose degrading genes into an organism that natively does not contain them, this invention extends metabolic pathways beyond those present natively in the prokaryotic organism itself for the purpose of producing ethanol.

According to a first aspect of the present invention, there is provided polynucleotide sequences and their corresponding polypeptide sequences from microorganisms that allow for the expression of cellulose hydrolytic proteins in bacteria for the purpose of producing an alcohol or alcohol precursor. Gene and promoter sequences were introduced into the prokaryotic cell using techniques such as two-step allelic exchange or using expression vectors. Natural promoters and ribosomal binding sites (RBS) from the bacterial expression strain were attached upstream of the gene's start codon to induce gene expression within the bacterium. Methods to transfer these genes from cloning vectors into the organisms of interest are also briefly outlined and are modelled after the protocol seen for the knock in or knock out of genes in a different bacterium, Pseudomonas aeruginosa, using the aforementioned two-step allelic exchange, however these defined methods are not meant to limit the scope of the invention and thus CRISPR and other undefined cloning methods can also be used.

According to a preferred embodiment of the present invention, there is provided a genetically modified ethanologenic organism which comprises a β-glucosidase 1 (bgl1) polynucleotide sequence, wherein said bgl1 polynucleotide sequence has at least 70% sequence coverage to SEQ 3 or SEQ 14, and at least 70% sequence identity to SEQ 3 or SEQ 14.

According to a preferred embodiment of the present invention, there is provided a genetically modified ethanologenic organism which comprises a β-glucosidase 1 (bgl1) polynucleotide sequence, wherein said bgl1 polynucleotide sequence is selected from the group consisting of: SEQ 3; SEQ 14; SEQ 15; SEQ 16; SEQ 17; SEQ 18; SEQ 19; SEQ 20; and SEQ 21.

According to a preferred embodiment of the present invention, SEQ 3 or SEQ 14, upon transcription and translation, provides a β-glucosidase 1 (BGL1) polypeptide sequence SEQ 4. According to a preferred embodiment of the present invention, there is provided a genetically modified ethanologenic organism which comprises a BGL1 polypeptide sequence which has at least 70% sequence coverage to SEQ 4, and at least 35% sequence identity to SEQ 4.

According to a preferred embodiment of the present invention, there is provided a genetically modified ethanologenic organism which comprises a β-glucosidase 1 (BGL1) polypeptide sequence wherein said BGL1 polypeptide sequence is selected from the group consisting of: SEQ 4; SEQ 22; SEQ 23; SEQ 24; SEQ 25; SEQ 26; SEQ 27; SEQ 28; SEQ 29; SEQ 30; SEQ 31; SEQ 32; SEQ 33; SEQ 34; SEQ 35; SEQ 36; SEQ 37; SEQ 38; SEQ 39; and SEQ 40.

According to a preferred embodiment of the present invention, there is provided a genetically modified ethanologenic organism which comprises an exoglucanase (cex-like) polynucleotide sequence, wherein said cex-like polynucleotide sequence has at least 70% sequence coverage to SEQ 1 or SEQ 68, and at least 70% sequence identity to SEQ 1 or SEQ 68.

According to a preferred embodiment of the present invention, there is provided a genetically modified ethanologenic organism which comprises an exoglucanase (cex-like) polynucleotide sequence, wherein said cex-like polynucleotide sequence is selected from the group consisting of: SEQ 1, SEQ 41, SEQ 42, SEQ 43, SEQ 44, SEQ 45, SEQ 46, SEQ 47, SEQ 48, and SEQ 68.

According to a preferred embodiment of the present invention, SEQ 1 or SEQ 68, upon transcription and translation, provides an exoglucanase (CEX-like) polypeptide sequence. According to a preferred embodiment of the present invention, there is provided a genetically modified ethanologenic organism which comprises a CEX-like polypeptide sequence which has at least 70% sequence coverage to SEQ 2, and at least 35% sequence identity to SEQ 2.

According to a preferred embodiment of the present invention, there is provided a genetically modified ethanologenic organism which comprises an exoglucanase (CEX-like) polypeptide sequence, wherein said CEX-like polypeptide sequence is selected from the group consisting of: SEQ 2; SEQ 49; SEQ 50; SEQ 51; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; and SEQ 67.

According to a preferred embodiment of the present invention, there is provided a genetically modified ethanologenic organism which comprises:
an exoglucanase (cex-like) polynucleotide sequence, wherein said cex-like polynucleotide sequence has at least 70% sequence coverage to SEQ 1 or SEQ 68, and at least 70% sequence identity to SEQ 1 or SEQ 68; and
a β-glucosidase 1 (bgl1) polynucleotide sequence, wherein said bgl1 polynucleotide sequence has at least 70% sequence coverage to SEQ 3 or SEQ 14 and at least 70% sequence identity to SEQ 3 or SEQ 14.

Preferably, said organism comprises:
an exoglucanase (cex-like) polynucleotide sequence selected from the group consisting of: SEQ 1; SEQ 41; SEQ 42; SEQ 43; SEQ 44; SEQ 45; SEQ 46; SEQ 47; SEQ 48; and SEQ 68; and
a β-glucosidase 1 (bgl1) polynucleotide sequence selected from the group consisting of: SEQ 3; SEQ 14; SEQ 15; SEQ 16; SEQ 17; SEQ 18; SEQ 19; SEQ 20; and SEQ 21.

According to a preferred embodiment of the present invention, there is provided a genetically modified ethanologenic organism which upon transcription and translation under the control of the native of a native or synthetic promoter and Ribosomal binding site (RBS), comprises:
an exoglucanase (CEX-like) polypeptide sequence, wherein said CEX-like polypeptide sequence has at least 70% sequence coverage to SEQ 2, and at least 35% sequence identity to SEQ 2; and
a β-glucosidase 1 (BGL1) polypeptide sequence, wherein said BGL1 polypeptide sequence has at least 70% sequence coverage to SEQ 4, and at least 35% sequence identity to SEQ 4.

Preferably, said organism comprises:
said exoglucanase (CEX-like) polypeptide sequence is selected from the group consisting of: SEQ 2; SEQ 49; SEQ 50; SEQ 51; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; and SEQ 67; and
said β-glucosidase 1 (BGL1) polypeptide sequence is selected from the group consisting of: SEQ 4; SEQ 22; SEQ 23; SEQ 24; SEQ 25; SEQ 26; SEQ 27; SEQ 28; SEQ 29; SEQ 30; SEQ 31; SEQ 32; SEQ 33; SEQ 34; SEQ 35; SEQ 36; SEQ 37; SEQ 38; SEQ 39; and SEQ 40.

Preferably, said organism further comprises a promoter and RBS sequence which drives gene expression, wherein the genetic material for the promoter/RBS sequences comprises at least one or another of the following: SEQ 5; SEQ 6; SEQ 7; SEQ 8; SEQ 9; SEQ 10; SEQ 11; SEQ 12; and SEQ 13. Preferably, the organism is a bacterium. More preferably, the organism is selected from the group consisting of the genera Aspergillus, Mucor, Zymomonas, Escherichia, Clostridia, Bacillus, and Pseudomonas. According to a preferred embodiment of the present invention, the organism is a prokaryotic organism. Preferably, the organism belongs to the bacterial genus Zymomonas.

According to a preferred method of the present invention, the ethanologenic organism is selected from the genus of Aspergillus, Mucor, Zymomonas, Escherichia, Clostridia, Bacillus, and Pseudomonas, however this list by no means is meant to limit the scope of the invention. Preferably, the ethanologenic organism is the bacteria Zymomonas mobilis.

According to another aspect of the present invention, ethanologenic bacterial cells were genetically modified by inserting a bacterial promoter sequence into the bacterial genome, immediately followed by a cellulose hydrolytic gene(s)-related to the degradation of cellulose. In some embodiments, the native promoter and polynucleotide sequence could also include the promoter and ribosomal binding site (RBS) including various combinations from the following genes: Glyceraldehyde-3-phosphate dehydrogenase, type I (gap) (P_{gap}), Thiamine pyrophosphate protein TPP binding domain-containing protein (pdc) (P_{pdc}), the EF-Tu transcription factor (P_{tuf}), Phosphopyruvate hydratase (eno) (Pₑₙₒ), 2-Dehydro-3-deoxyphosphogluconate aldolase/4-hydroxy-2-oxoglutarate aldolase (eda) (P_{eda}), glucose-6-phosphate dehydrogenase (zwf) (P_{zwf}), ROK family protein (frk) (P_{frk}), Carboxymethylenebutenolidase (clcD1) (P_{clcD1}), as well as Glucosamine-fructose-6-phosphate aminotransferase (glmS) (P_{glmS}), however this list of possible promoters is in no way meant to limit the scope of the invention. In preferred embodiments, the promoter and RBS polynucleotide sequence to regulate cellulose degradation for the production of an alcohol or alcohol precursor is the native promoter for the Glyceraldehyde-3-phosphate, dehydrogenase, type 1 (gap) gene (P_{gap}), obtained from Z. mobilis.

According to a preferred embodiment of the present invention, cex-like genes can be acquired from, but by no means is limited to, the following prokaryotic species: Cellulomonas uda, Cellulomonas palmilytica, Cellulomonas cellasea, Saccharothrix syringae, Promicromonospora iranensis, Glycomyces paridis, Micromonospora fulviviridis, Couchioplanes caeruleus, Streptomonospora alba, Phytoactinopolyspora halotolerans, Cellulomonas wangsupingiae, Xylanimonas cellulosilytica, Actinoplanes lutulentus, or Micromonospora saelicesensis. These listed organisms are exemplary only and are in no way meant to limit what organisms these genes can be acquired from, nor to limit the scope of the invention.

According to a preferred embodiment of the present invention, bgl1 genes can be acquired from, but by no means is limited to, the following eukaryotic species: Aspergillus niger, Aspergillus luchuensis, Penicillium vulpinum, Sanghuangporus baumii, Penicillium rolfsii, Trichoderma gamsii, Halenospora varia, Daldinia childiae, Fusarium solani, Glaciozyma antarctica, Monascus purpureus, and Trichophyton interdigitale. These listed organisms are exemplary only and are in no way meant to limit what organisms these genes can be acquired from, nor to limit the scope of the invention.

According to a preferred embodiment of the present invention, the cex-like and bgl1 genes are obtained from the organism Cellulomonas uda (cex-like) and Aspergillus niger (bgl1), respectively. In other embodiments, a native promoter sequence can be used to bolster the production of proteins and/or molecules utilized in the production of an alcohol or alcohol precursor through the hydrolysis of cellulose.

According to another embodiment of the present invention, the ethanologenic bacterial cells have been genetically modified to have improved degradation of cellulose through the addition of both prokaryotic and eukaryotic gene(s) which when transcribed and translated generate cellulose hydrolytic enzymes. In some embodiments, the transcription of these genes is driven by a promoter sequence, and can include, but is not limited to, any combination of P_{gap}-cex-like or P_{gap}-bg11 gene cassettes which have been integrated into a bacterial cell.

### BRIEF DESCRIPTION OF THE ACCOMPANYING FIGURE

The invention may be more completely understood in consideration of the following description of various embodiments of the invention in connection with the accompanying FIGURE, in which:

FIG. 1 is a schematic representation of the structure of cellulose.

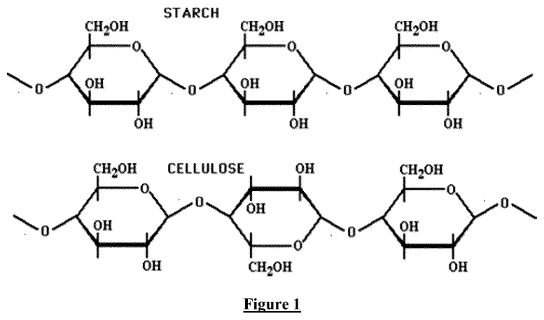

### DETAILED DESCRIPTION OF THE INVENTION

It is known to the person skilled in the art that some species of ethanologenic bacteria contain and express endogenous endoglucanase and endogenous exoglucanase enzymes with functional cellulase activities. According to an aspect of the present invention, there is provided genetic modifications to ethanologenic bacterial strains to enhance such innate capabilities and thereby increase the efficiency of cellulose degradation by said bacteria.

According to a preferred embodiment of the present invention, there are provided genetic modifications to ethanologenic bacterial strains which allow for the degradation of cellulose with the purpose of producing an alcohol or alcohol precursor from an inexpensive cellulosic feedstock using a bacterial species modified with a cex-like polynucleotide sequence (such as SEQ 1 or SEQ 68) or, upon transcription and translation of said cex-like polynucleotide, a CEX-like polypeptide (such as SEQ 2).

According to a preferred embodiment of the present invention, there are provided genetic modifications to ethanologenic bacterial strains which allow for the degradation of cellulose with the purpose of producing an alcohol or alcohol precursor from an inexpensive cellulosic feedstock using bacterial species modified with a bgl1 polynucleotide sequence (such as SEQ 3 or SEQ 14) or, upon transcription and translation of said bgl1 polynucleotide, a BGL1 polypeptide (such as SEQ 4).

According to an aspect of the present invention, there is provided genetic modifications to ethanologenic bacterial strains which includes a combination of cex-like and bgl1 polynucleotide sequences allowing for increased degradation of cellulose with the intent to produce an alcohol or alcohol precursor from inexpensive feedstock as a source of cellulose.

### Definitions

Within the context of this invention all terms and technical parameters described fall within their commonly known meanings as known by individuals within the field of science that the proposed invention is associated with unless otherwise stated. Furthermore, unless otherwise indicated, all techniques utilized within the context of this invention are commonly conducted within the fields of molecular biology, cell biology, biochemistry, and microbiology.

An alcohol or alcohol precursor is any molecule that is obtained as an intermediate, by-product, or co-product of the conversion of a cellulosic material to an alcohol. It is known to the person skilled in the art that said alcohol precursor can be an oligosaccharide, di-saccharide, monosaccharide, organic acid and their corresponding salts, aldehyde, ketone, etc.

Biofuels within the context of this invention is defined as any fuel for which is produced using biological material.

Bioethanol within the context of this invention is defined as ethanol that is produced from lignocellulosic biomass.

Cellulosic ethanol is defined within the context of this invention as ethanol that is produced through the degradation of cellulose.

Cellobiose is defined within the context of this invention as the dimer of glucose formed from the degradation of cellulose.

Cellulose hydrolysis within the context of this invention is defined as the degradation of cellulose via hydrolytic processes.

Hydrolysis within the context of this invention is defined as a chemical or microbiological reaction which facilities the breaking of chemical bonds between molecules.

A monomer is defined within this invention as a single molecule that can, under specific conditions, be combined with other molecules, including itself, to generate more complex structures called polymers.

A polymer is defined within the context of this invention as a material comprised of repeating subunits of the same or different molecule.

A biopolymer is defined within the context of this invention as a polymer that is typically produced by the cells of living organisms. By way of example, cellulose is considered a biopolymer.

A polynucleotide within the context of this invention is defined as the collection of individual nucleotides in any organization or size.

A polypeptide within the context of this invention is defined as the combination of multiple peptides of any organization or size that relates to the amino acid sequence. The term polypeptide and protein within the context of this invention can be used interchangeably.

A vector within the context of this invention refers to the composition of a polynucleotide within the intended purpose of introducing nucleic acids into one or more organism types. Vectors are further defined based on their functional purpose and can be designated as expression vectors, cloning vectors, plasmids, or shuttle vectors.

The term expression within the context of this invention refers to the generation of a polypeptide sequence which is produced based on its polynucleotide sequence or gene.

The term promoter in the context of this invention is used to describe the nucleic acid sequence for the regulation and binding of polymerases for the purpose of the transcribing of a gene. This promoter can be native to an organism, or a non-endogenous promoter can be introduced into an organism to alter the regulation of gene expression.

The term gene refers to a sequence of DNA that encodes for a specific polypeptide sequence. A gene can include both sequences between coding regions (introns) and the encoding sequence itself (exon).

The term recombinant in the context of this invention refers to the modification or alteration of a sequence associated with either a polypeptide or polynucleotide sequence. Recombination can be utilized for altering expression and coding segments of a gene of interest that would produce a non-native or non-naturally occurring product.

The term homology or homologous refers to the level of similarity between two or more polypeptide or polynucleotide sequences.

The terms transfection, transformation, or introduced refer to the addition of polynucleotide sequence(s) that would normally be considered exogenous to organism. This may include the addition of a polynucleotide directly to the genome of an organism or the transfer of a plasmid and/or vector.

Within the context of this patent the terms native or natural refers to polypeptide and/or polynucleotides present within the organism prior to any modification. These native or naturally occurring polypeptides and/or polynucleotides would be present or produced by the organism without any external alterations.

The term metabolic pathway refers to the subsequential biochemical reactions involved in the formation of a biologically relevant product within an organism.

Within this invention the terms knock-in and knock-out refer to the addition or removal of DNA sequences within an organism and can also be interchangeable with the terms insertion and deletion, respectively.

The term promoter within the context of this invention refers to a sequence of DNA that is responsible for the initiation of DNA transcription and thus polypeptide formation.

A coding sequence within the context of this invention refers to a sequence of polynucleotides or DNA that facilitates the generation of a protein through transcription and translational processes.

Genetic modification or related statements herein refer to the alteration of the genetic code of an organism which includes the insertion or deletion of DNA sequences within an organism. Within the context of this invention, genetic modification could include insertion and maintenance of an expression vector into the organism, or the direct modification of the organism's genome by directly adding or deleting genes through a process like two-step allelic exchange or CRISPR.

The term ribosomal binding site (RBS) refers to the region within a polynucleotide sequence that allows for the appropriate binding of the ribosome to facilitate the translation of a polynucleotide sequence to produce the corresponding polypeptide sequence, which includes the terms protein, enzyme, and plasmid.

The term cloning vector herein refers to a polynucleotide sequence or plasmid that can be replicated within a host organism for storage or amplification purposes. A cloning vector may contain all the necessary regulatory sequences needed to facilitate the transcription and translation of a protein.

The term unmodified promoter is defined as a promoter sequence which has been unaltered or exists within the host organism itself.

The term two-step allelic exchange is referred to the process by which a gene of interest is either interested or deleted from an organism through specific selective conditions. The insertion or deletion of a specific gene of interest is done so through the utilization of distinct polynucleotide sequences which allow for the exchange of genetic material between two sources.

The term CRISPR cloning is defined as a process by which the gene of interest is inserted or removed from an organism's genome using the CRISPR-CAS9 cloning system.

The terms upstream and downstream refer to regions of polynucleotides which are found prior to or after a specific gene of interest within a plasmid and/or genome of an organism.

The term enzyme within this invention defines a polypeptide sequence, specifically in the form of a protein, that can modify a biological molecule or take part within its generation through direct or indirect interactions. The process by which an enzyme influences the modification and/or production of a biological molecule and/or product is termed enzymatic activity.

The term open reading frame (ORF) refers to the collection of nucleotides which are found in between the start and stop codons of a polypeptide encoding DNA sequence.

The term codon(s) refers to 3 adjacent nucleotides in a polynucleotide sequence that are used by the cell to “decode” the polynucleotide sequence when the polynucleotide sequence is translated to make the polypeptide sequence and are responsible defining the order of protein residues in a polypeptide sequence based on this code. Based on a 3-letter code, and 4 different nucleotide bases, these codons include 64 different combinations that are able to be used by the cell, which with some redundancy codes for 22 possible protein residues, as well as 1 start and 3 stop codons.

A start and stop codon refer to a nucleotide codon sequence comprised of three specific nucleotides in succession of each other which allows for the identification of the initiation (start) and termination (stop) for the transcription of a polypeptide sequence.

The term encoded refers to the polypeptide sequence that is obtained from a polynucleotide sequence after the polynucleotide sequence had been transcribed and translated.

The combined term transcribed and translated (or the combined process of transcription and translation) refers to the process by which a polynucleotide sequence is used to produce a polypeptide sequence.

Metabolic engineering herein refers to the alteration of an organism's metabolic pathway potential. This can include both the deactivation and/or altering of pre-existing metabolic pathways of an organism or the inclusion of additional metabolic processes.

Sequence alignment herein refers to a bioinformatic technique by which two polynucleotide sequences or two polypeptide sequences are arranged or aligned in such a way as to identify regions of similarity between a reference sequence (the sequence that is known) and the quarry sequence (the sequence to be compared to the reference sequence). Those skilled in the art know that alignment algorithms such as, but by no means limited to, the BLAST, ALIGN, or CLUSTAL algorithms can be used to obtain this information for polynucleotide or polypeptide sequences, respectively.

Percentage sequence identity or percentage identity herein refers to the similarity between 2 sequences that have been processed through a sequence alignment, to provide insight into how similar aligned sequences are at either the nucleotide or peptide level for polynucleotide or polypeptide sequences, respectively. The percentage identity is used to determine the similarity of a query sequence to a reference sequence.

Percentage sequence coverage or percentage coverage refers to the number of aligned nucleotides or peptides in a query sequence relative to the length of the reference sequence. The percentage coverage provides an indication of how much of the reference polynucleotide or polypeptide sequence is covered by the query sequence, allowing for instance the lengths of the found genes or proteins to be compared. The BLASTN algorithm was used herein as one method to determine the percentage identity and percentage coverage between one or even multiple different polynucleotide sequences with respect to an inputted reference sequence, allowing for the determination of the percentage identity and percentage coverage of one or many query sequences to said reference sequence. One of ordinary skill in the art will recognize that search results from a BLASTN search will be influenced by the search parameters used in the search. Therefore, for all BLASTN searches done with respect to this invention to identify other sequences which have been catalogued in the NCBI polynucleotide databases relative to a reference include the following parameters:
Search set parameters are comprising of “standard databases (nr ect)”, with the specific database used being the “Nucleotide collection (nr/nt)”, and no exclusions or limitations were placed on the search (all default parameters)
Program selection algorithm parameters includes the highly similar sequences (known as the megablast algorithm) (the default parameter)
Algorithm parameters altered include the Max target sequences, which was set at 5000, otherwise all default parameters are used for relevant searches (other parameters in “General parameters”, and all parameters in “Scoring parameters” and “Filters and masking” are default)

The BLASTP algorithm was used herein as one method to determine the percentage identity and percentage coverage between one or even multiple different polypeptide sequences with respect to an inputted reference sequence, allowing for the determination of the percentage identity and percentage coverage of one or many query sequences to said reference sequence. One of ordinary skill in the art will recognize that search results from a BLASTP search will be influenced by the search parameters used in the search. Therefore, for all BLASTP searches done with respect to this invention to identify other sequences which have been catalogued in the NCBI polypeptide databases relative to a reference include the following parameters:
Search set parameters are comprising of “standard databases (nr ect)”, with the specific database used being the “Non-redundant protein sequences (nr)”, and no exclusions or limitations were placed on the search (all default parameters)
Program selection algorithm parameters includes the BLASTP (known as the protein-protein BLAST algorithm) (the default parameter)
Algorithm parameters altered include the Max target sequences, which was set at 5000, otherwise all default parameters are used for relevant searches (other parameters in “General parameters”, and all parameters in “Scoring parameters” and “Filters and masking” are default). Notable default parameters include an “Expect Threshold and word size of 0.05 and 5, respectively in the general parameters, the usage of the BLOSUM62 matrix with gap costs of Existence:11 and Extension: 1 for the Scoring parameters, and no filter or masking components selected.

The phrases substantially similar or substantially identical in the context of at least 2 nucleic acid sequences or at least 2 polypeptide sequences typically means that a polynucleotide, polypeptide, or region or domain of a polypeptide has a percentage coverage of at least 70%, 75%, 80%, 85%, 90%, 95%, 97%, 99%, or even 99.5%, and at least 25%, 30%, 35%, 40%, 45%, 50%, 55%, 60%, 65%, 70%, 75%, 80%, 85%, 86%, 87%, 88%, 89%, 90%, 91%, 92%, 93%, 94%, 95%, 96%, 97%, 98%, 99%, or even 99.5% percentage identity to the reference sequence. Some polynucleotide or polypeptide sequences that fall in this category are sequences that share genetic or protein homology to the reference sequence.

The terms genetic and protein homology, or homologous sequences refer to polynucleotide sequences or translated polypeptide sequences that have a similar or identical function in the cell. For example, 2 different proteins share a similar or identical function even though they were isolated from 2 different organisms. Polynucleotide sequences with homology are generally understood to have similar or identical biochemical functionality.

The term endoglucanase refers to a protein with a certain enzymatic activity to degrade cellulose by attacking random internal 1,4-β-linkages between the glucose units in cellulose.

The term exoglucanase refers to a protein with a certain enzymatic activity to degrade cellulose by releasing cellobiose or glucose from the nonreducing end of the cellulose polymer.

The term glucosidase refers to a protein with enzymatic activity to degrade cellobiose to glucose.

The term ethanologenic organism refers within this invention to an organism that can produce ethanol as part of its native metabolic processes.

### Enzymes and Promoters for Cellulosic Ethanol Production

Example polypeptide sequences for enzymes involved in cellulose degradation that can be integrated into prokaryotic organisms are provided in the sequence listing. The expression and production of these sequences within the cell are partially driven by the genetic polynucleotide promoter and ribosomal binding site sequences as provided in the sequence listings: SEQ 5 (P_{gap}), SEQ 6 (P_{pdc}), SEQ 7 (P_{tuf}), SEQ 8 (P_{enc}), SEQ 9 (P_{eda}), SEQ 10 (P_{zwf}), SEQ 11 (P_{frk}), SEQ 12 (P_{clcD1}), and SEQ 13 (P_{glms}). The invention is not limited to the use of these polynucleotide sequences and their respective polypeptide sequences. Those of ordinary skill in the art know that organisms of a wide variety of species commonly express and utilize homologous proteins, which contain insertions, substitutions and deletions in the polypeptide sequences of the polynucleotide sequences listed above, and effectively provide a similar function. For example, the protein sequences for CEX-like from Cellulomonas uda or Couchioplanes caeruleus or Promicromonospora iranensis; or BGL1 from Aspergillus niger or Penicillium vulpinum or Halenospora varia may differ to different degrees from the polypeptide sequences seen between these organisms yet maintain similar or identical functions of the protein within the organism with respect to enzymatic function. Protein sequences comprising such variations are included within the scope of the present invention and are considered substantially or sufficiently similar to the referenced polynucleotide sequences above and upon transcription and translation their respective polypeptide sequences. Although it is not intended that the present invention is limited by any theory by which it achieves its advantageous result, it is believed and supported by biochemical knowledge that the identity between polypeptide sequences that is necessary to maintain proper functionality is related to maintaining the tertiary structure (3D) of the polypeptide. This maintenance of the tertiary structure is associated with the specific interactive/catalytic portions of the protein sequence and will therefore have the desired activity, and it is contemplated that a protein including these interactive sequences in the proper special context will have this activity.

The person of ordinary skill in the art knows that many different amino acids contain properties between each other and can serve similar functions in the final polypeptide sequence. Thus, when one amino acid is changed with another amino acid from this group, such as a non-polar amino acid, an uncharged polar amino acid, a charged polar acidic amino acid, or a charged polar basic amino acid, some polypeptide functionality is generally maintained. For example, it is known that the uncharged polar amino acid serine may be substituted for the uncharged polar amino acid threonine in a polypeptide without substantially altering the protein structure and functionality. Whether a given substitution will affect the functionality of the enzyme may be determined without undue experimentation using synthetic techniques and screening assays known to one of ordinary skill in the art.

The person of ordinary skill in the art will recognize that changes in the protein sequence, resulting from individual single or multi-nucleotide substitutions, deletions, or additions to a polynucleotide will lead to changes in the resulting translated polypeptide sequence. Small mutations, such as the change of an amino acid from one to another, or the addition or elimination of single amino acids, or a small to moderate percentage of amino acids from the encoded polypeptide sequence can be considered “sufficiently similar” when the alteration results in the substitutions of an amino acid with a chemically similar amino acid. Thus, any number of amino acid residues in a polypeptide chain, selected from a group of integers from 1-50, can be so altered. Thus, for example, 1, 2, 3, 5, 10, 12, 20, 32, 41, or even 50 alterations can be made. Conservatively modified variants typically provide similar biological activity to the unmodified variants and typically provide similar biological activity as the unmodified polypeptide sequence from which they are derived. For example, modification of CEX-like and BGL1 to yield functional proteins generally have a sequence identity of at least 40%, 50%, 60%, 70%, 80%, or 90%, preferably a sequence identity of greater than 50%, of the native protein to allow processing of its native substrate. Tables of conserved substitution provide lists of functionally similar amino acids. Amino acids in polypeptide chains that are similar to one another include the following groups: (1) Serine (S), Threonine (T); (2) Aspartic acid (D), Glutamic acid (E); (3) Asparagine (N), Glutamine (Q); (4) Alanine (A), Leucine (L), and Isoleucine (I).

### Suitable Polynucleotide and Polypeptide Sequences for CEX-Like and BGL1

The person of ordinary skill in the art will recognize that many different organisms will have functionally similar polynucleotide and polypeptide sequences (or homology between the sequences), however there may be differences between these sequences when compared to a reference sequence. As examples, suitable polynucleotides and their corresponding polypeptide sequences for cellulose degradation can be seen below. Note that the following sequences by no means are meant to limit the scope of the invention. In fact, any substantially similar polynucleotide sequences or substantially similar produced polypeptide sequences for the CEX-like and BGL1 genes/proteins with similar function or similarity to these genes/proteins in the cellulose degradation pathway can also be used for producing alcohol and alcohol precursors including cellulosic ethanol production.

According to a preferred embodiment of the present invention, the β3-glucosidase 1 (bgl1) polynucleotide sequence SEQ 3 or SEQ 14 is one β-glucosidase utilized for producing alcohol and alcohol precursors including cellulosic ethanol production. In this embodiment, bgl1 is under the transcriptional control of a native promoter and a ribosomal binding site. However, in other embodiments of the invention, bgl1 polynucleotide sequences that are homologous and/or substantially similar to SEQ 3 or SEQ 14 may also be used in the present invention to produce cellulosic ethanol. The bgl1 polynucleotide sequences for β-glucosidase 1 in these embodiments will have at least 70% sequence coverage, or more preferably greater than 75%, 85%, 90%, 95%, 97% or most preferentially greater than 99% sequence coverage to SEQ 3 or SEQ 14, and sequence identities of at least 70%, or more preferentially greater than 75%, 80%, 90%, or 95% sequence identity, and most preferentially greater than 99% sequence identity to SEQ 3 or SEQ 14. The bgl1 polynucleotide sequences may include, but by no means are limited to, the following sequences: SEQ 3; SEQ 14; SEQ 15; SEQ 16; SEQ 17; SEQ 18; SEQ 19; SEQ 20; and SEQ 21.

According to a preferred embodiment of the present invention, the β3-glucosidase 1 (BGL1) polypeptide SEQ 4 is utilized to produce producing alcohol and alcohol precursors including cellulosic ethanol by the cell, whereby SEQ 4 is produced from the transcription and translation of the β3-glucosidase 1 (bgl1) polynucleotide SEQ 3 or SEQ 14. However, in other embodiments of the invention, BGL1 polypeptide sequences that are homologous and substantially similar to SEQ 4 may also be used in the present invention to produce cellulosic ethanol. BGL1 polypeptide sequences in these embodiments will have at least 70% sequence coverage to SEQ 4, or more preferentially greater than 75%, 80%, 85%, 90%, 95%, 97%, 98%, or most preferentially greater than 99% sequence coverage to SEQ 4, and a sequence identity of at least 35% to SEQ 4, or more preferably greater than 40%, 45%, 50%, 60%, 70%, 75%, 80%, 85%, 90%, 95%, or 97% sequence identity, or most preferentially greater than 99% sequence identity to SEQ 4. These polypeptide sequences may include, but are by no means limited to, the following sequences: SEQ 4; SEQ 22; SEQ 23; SEQ 24; SEQ 25; SEQ 26; SEQ 27; SEQ 28; SEQ 29; SEQ 30; SEQ 31; SEQ 32; SEQ 33; SEQ 34; SEQ 35; SEQ 36; SEQ 37; SEQ 38; SEQ 39; and SEQ 40.

According to a preferred embodiment of the present invention, the 1,4-beta-cellobiohdryolase (cex-like) polynucleotide sequence SEQ 1 or SEQ 68 is one exoglucanase utilized for producing alcohols and alcohol precursors including cellulosic ethanol production. In this embodiment, cex-like is under the transcriptional control of a native promoter and a ribosomal binding site. However, in other embodiments of the invention, cex-like polynucleotide sequences that are homologous and/or substantially similar to SEQ 1 or SEQ 68 may also be used in the present invention to produce cellulosic ethanol. The cex-like polynucleotide sequences for 1,4-beta-cellobiohydrolase in these embodiments will have at least 70% sequence coverage, or more preferably greater than 75%, 80%, 85%, 90%, 95%, 98% or most preferentially greater than 99% sequence coverage to SEQ 1 or SEQ 68, and sequence identities of at least 70%, or more preferentially greater than 75%, 80%, 90%, or 95% sequence identity, and most preferentially greater than 99% sequence identity to SEQ 1 or SEQ 68. These cex-like polynucleotide sequences may include, but by no means are limited to, the following sequences: SEQ 1; SEQ 41; SEQ 42; SEQ 43; SEQ 44; SEQ 45; SEQ 46; SEQ 47; SEQ 48; and SEQ 68.

According to a preferred embodiment of the present invention, the exoglucanase (CEX-like) polypeptide SEQ 2 is utilized to produce producing alcohols and/or alcohol precursors including cellulosic ethanol by the cell, whereby SEQ 2 is produced from the transcription and translation of the exoglucanase (cex-like) polynucleotide SEQ 1 or SEQ 68. However, in other embodiments of the invention, CEX-like polypeptide sequences that are homologous and substantially similar to SEQ 2 may also be used in the present invention to produce cellulosic ethanol. The CEX-like polypeptide sequences for exoglucanase in these embodiments will have at least 70% sequence coverage to SEQ 2, or more preferentially greater than 75%, 80%, 85%, 90%, 95%, 97%, 98%, or most preferentially greater than 99% sequence coverage to SEQ 2, and a sequence identity of at least 35% to SEQ 2, or more preferably greater than 40%, 45%, 50%, 60%, 70%, 75%, 80%, 90%, or 95% sequence identity, or most preferentially greater than 99% sequence identity to SEQ 2. These CEX-like polypeptide sequences may include, but are by no means limited to, the following sequences: SEQ 2; SEQ 49; SEQ 50; SEQ 51; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; and SEQ 67.

Embodiments of this invention are described herein, including the best mode known to the inventors for carrying out the invention. Variations of those embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

### Sequences:

SEQ 1:
Polynucleotide sequence of exoglucanase
(cex-like) (Cellulomonas uda)
ATGACCCCCTCATCCATGTCCAGACGCGCGCGCGTCGCGTCCGCG
CTCGCGATCGTCACGCTCGGCGCGACCATCGCGACGACGATCCCC
GCCCAGGCCGCCGGCAGCACGCTGCAGGCCGCGGCCTCCGAGAGC
GGCCGGTACTTCGGTACCGCCATCGCGGCGTTCAAGCTCAACGAC
AGCACGTACTCGTCCATCGCGAACCGTGAGTTCAACATGATCACG
GCCGAGAACGAGATGAAGATGGACGCGACGGAGCCGTCGCAGAAC
AACTTCAGCTACTCCAGCGGCGACCAGATCCTCAACTGGGCGCGC
AGCAACGGCAAGCGGGTTCGTGGGCACGCGCTCGCGTGGCACTCG
CAGCAGCCGGGCTGGATGCAGAACATGTCCGGCACCCAGCTGCGC
AACGCGATGCTCAACCACGTCACTCAGGTCGCGACGCACTACAAG
GGCAAGATCTACGCCTGGGACGTCGTGAACGAGGCGTACGCGGAC
AGCGGCGGCGGCCGTCGCGACTCGAACCTGCAGCGCACCGGCGAC
GACTGGATCGAGGCGGCGTTCCGCGCGGCCCGCGCCGCGGACCCG
GGCGCCAAGCTCTGCTACAACGACTACAACACGGACAACTGGACC
TGGGCCAAGACGCAGGGCGTCTACAACATGGTCAAGGACTTCAAG
GCCCGTGGGGTGCCGATCGACTGCGTCGGTTTCCAGTCACACTTC
AACTCGGGCAGCCCGTACCCGAGCAACTACCGGACCACGCTGCAG
AACTTCGCCGCGCTCGGCGTCGAGGTGCAGATCACCGAGCTCGAC
ATCGAGGGCTCGGGCCAGCAGCAGGCCCAGACGTACGCCAACGTG
GTCGCCGACTGCCTCGCCGTGAAGGCCTGCACCGGCATCACGGTG
TGGGGCGTGCGCGACTCCGACTCGTGGCGCTCCTCGGGTACCCCG
CTGCTGTTCGACGGCTCGGGCAACAAGAAGGCCGCGTACACCTCC
ACGCTCGACGCGCTGAACCGCGGCGGCGTCCCGACGGACCCGACG
ACGCCGCCCACGGACCCGACCACGCCGCCCACGGACCCGACGACC
CCGCCCACGGACCCGACCACCCCGCCGACCGACCCCACCACGCCT
CCGACCGACCCGACGGGCCGGTGCACGGCGAGCCTGGCGATCGCG
AACGCGTGGCCCGGCGGGTACCAGGCGACCGTGACCGTCAAGGCG
GGCTCGTCGTCGATCAACGGCTGGCGCGTCACGCTGCCCAGCGGT
GTCAGCACGAGCAACCTCTGGAACGGCGTGCTCGCCAACGGTGTG
GTGACCAACGCGCCGTACAACGGCTCGGTCGGCGCCGGCCAGTCG
ACGACCTTCGGGTTCGTCGGCAACGGCAGCGCTCCGAGCGCAGGC
TCCGTGACCTGCGCCTGA
SEQ 2:
Polypeptide sequence of exoglucanase (CEX-like)
(Cellulomonas uda)
MTPSSMSRRARVASALAIVTLGATIATTIPAQAAGSTLQAAASES
GRYFGTAIAAFKLNDSTYSSIANREFNMITAENEMKMDATEPSQN
NFSYSSGDQILNWARSNGKRVRGHALAWHSQQPGWMQNMSGTQLR
NAMLNHVTQVATHYKGKIYAWDVVNEAYADSGGGRRDSNLQRTGD
DWIEAAFRAARAADPGAKLCYNDYNTDNWTWAKTQGVYNMVKDFK
ARGVPIDCVGFQSHFNSGSPYPSNYRTTLQNFAALGVEVQITELD
IEGSGQQQAQTYANVVADCLAVKACTGITVWGVRDSDSWRSSGTP
LLFDGSGNKKAAYTSTLDALNRGGVPTDPTTPPTDPTTPPTDPTT
PPTDPTTPPTDPTTPPTDPTGRCTASLAIANAWPGGYQATVTVKA
GSSSINGWRVTLPSGVSTSNLWNGVLANGVVTNAPYNGSVGAGQS
TTFGFVGNGSAPSAGSVTCA*
SEQ 3:
Polynucleotide sequence of β-glucosidase 1 (bgl1)
(Aspergillus niger)
ATGAGGTTCACTTTGATCGAGGCGGTGGCTCTGACTGCCGTCTCG
CTGGCCAGCGCTGATGAATTGGCCTACTCCCCTCCGTATTACCCC
TCCCCTTGGGCCAATGGCCAGGGTGACTGGGCGGAAGCATACCAG
CGCGCTGTTGATATCGTCTCGCAGATGACATTGGCTGAGAAGGTC
AATTTGACTACGGGAACTGGATGGGAATTGGAATTATGTGTTGGT
CAGACTGGAGGTGTTCCCCGATTGGGAATTCCGGGAATGTGTGCA
CAGGATAGCCCTCTGGGTGTTCGTGACTCCGACTACAACTCTGCG
TTCCCCGCCGGTGTCAACGTGGCCGCAACCTGGGACAAGAATCTG
GCTTACCTGCGTGGCCAGGCTATGGGTCAGGAGTTTAGTGACAAG
GGTGCTGATATCCAATTGGGTCCAGCTGCCGGCCCTCTCGGTAGA
AGTCCCGACGGCGGTCGTAACTGGGAGGGCTTCTCCCCCGACCCG
GCCCTCAGTGGTGTGCTCTTTGCAGAGACAATCAAGGGTATTCAG
GATGCTGGTGTGGTTGCAACGGCTAAGCACTACATCGCCTACGAG
CAGGAGCATTTCCGTCAGGCGCCTGAAGCTCAAGGCTACGGATTC
AATATTACCGAGAGTGGAAGCGCGAACCTCGACGATAAGACTATG
CATGAGCTGTACCTCTGGCCCTTCGCGGATGCCATCCGTGCAGGT
GCCGGTGCTGTGATGTGCTCGTACAACCAGATCAACAACAGCTAT
GGCTGCCAGAACAGCTACACTCTGAACAAGCTGCTCAAGGCTGAG
CTGGGTTTCCAGGGCTTTGTCATGAGTGATTGGGCGGCTCACCAT
GCCGGTGTGAGTGGTGCTTTGGCGGGATTGGACATGTCTATGCCG
GGAGACGTCGATTACGACAGTGGCACGTCTTACTGGGGTACCAAC
TTGACCATCAGTGTGCTCAACGGGACGGTGCCCCAATGGCGTGTT
GATGACATGGCTGTCCGCATCATGGCCGCCTACTACAAGGTCGGC
CGTGACCGTCTGTGGACTCCTCCCAACTTCAGCTCATGGACCAGA
GATGAATACGGCTTCAAGTACTACTATGTCTCGGAGGGACCGTAT
GAGAAGGTCAACCAGTTCGTGAATGTGCAACGCAACCATAGCGAG
TTGATCCGCCGTATTGGAGCAGACAGCACGGTGCTCCTCAAGAAC
GATGGCGCTCTTCCCTTGACTGGAAAGGAGCGCTTGGTCGCCCTT
ATCGGAGAAGATGCGGGTTCCAATCCTTATGGTGCCAACGGCTGC
AGTGACCGTGGGTGCGACAATGGAACATTGGCGATGGGCTGGGGA
AGTGGCACTGCCAACTTTCCCTACTTGGTGACCCCCGAGCAGGCC
ATCTCGAACGAGGTGCTCAAGAACAAGAATGGCGTATTCACTGCG
ACCGATAACTGGGCTATTGATCAGATTGAGGCGCTTGCTAAGACC
GCCAGTGTCTCTCTTGTCTTTGTCAACGCCGACTCTGGTGAGGGT
TATATCAATGTCGACGGAAACCTGGGTGACCGCAGGAACCTGACC
CTGTGGAGGAACGGCGACAATGTGATCAAGGCTGCTGCTAGCAAC
TGCAACAACACGATCGTTATTATTCACTCTGTCGGCCCAGTCTTG
GTTAACGAGTGGTACGACAACCCCAATGTTACCGCTATTCTCTGG
GGTGGTCTTCCCGGTCAGGAGTCTGGCAACTCCCTCGCCGACGTG
CTCTACGGCCGTGTCAACCCCGGTGCCAAGTCGCCCTTCACCTGG
GGCAAGACTCGTGAGGCCTACCAAGATTACTTGTACACCGAGCCC
AACAACGGCAACGGAGCGCCCCAGGAAGACTTCGTCGAGGGCGTC
TTCATTGACTACCGCGGATTTGACAAGCGCAACGAGACTCCTATC
TATGAGTTCGGCTATGGTCTGAGCTACACCACCTTCAACTACTCG
AACCTTCAGGTGGAGGTTCTGAGCGCCCCTGCGTACGAGCCTGCT
TCGGGCGAGACTGAGGCAGCGCCGACTTTCGGAGAGGTCGGAAAT
GCGTCGGATTACCTCTACCCCGATGGACTGCAGAGAATCACCAAG
TTCATCTACCCCTGGCTCAACAGTACCGATCTTGAGGCGTCTTCT
GGGGATGCTAGCTATGGGCAGGATGCCTCAGACTATCTTCCCGAG
GGAGCCACCGATGGCTCTGCGCAACCGATCCTGCCTGCCGGTGGT
GGTGCTGGCGGCAACCCTCGCCTGTACGACGAGCTCATCCGCGTG
ACGGTGACTATCAAGAACACCGGCAAGGTTGCGGGTGATGAAGTT
CCTCAACTGGTAAGTAGACAGCAAATTCGAACCAAGTCGGACCAA
GCTAATGAATCGCAGTATGTTTCTCTTGGCGGCCCTAACGAACCC
AAGATCGTGCTGCGTCAATTCGAGCGTATCACGCTGCAGCCGTCG
GAAGAGACGCAGTGGAGCACGACTCTGACGCGCCGTGACCTTGCG
AACTGGAATGTTGAGACGCAGGACTGGGAGATTACGTCGTATCCC
AAGATGGTGTTTGTCGGAAGCTCCTCGCGGAAGCTGCCGCTCCGG
GCGTCTCTGCCTACTGTTCACTAAGAATTCGCC
SEQ 4:
Polypeptide sequence of β-glucosidase 1 (BGL1)
(Aspergillus niger)
MRFTLIEAVALTAVSLASADELAYSPPYYPSPWANGQGDWAEAYQ
RAVDIVSQMTLAEKVNLTTGTGWELELCVGQTGGVPRLGIPGMCA
QDSPLGVRDSDYNSAFPAGVNVAATWDKNLAYLRGQAMGQEFSDK
GADIQLGPAAGPLGRSPDGGRNWEGFSPDPALSGVLFAETIKGIQ
DAGVVATAKHYIAYEQEHFRQAPEAQGYGFNITESGSANLDDKTM
HELYLWPFADAIRAGAGAVMCSYNQINNSYGCQNSYTLNKLLKAE
LGFQGFVMSDWAAHHAGVSGALAGLDMSMPGDVDYDSGTSYWGTN
LTISVLNGTVPQWRVDDMAVRIMAAYYKVGRDRLWTPPNFSSWTR
DEYGFKYYYVSEGPYEKVNQFVNVQRNHSELIRRIGADSTVLLKN
DGALPLTGKERLVALIGEDAGSNPYGANGCSDRGCDNGTLAMGWG
SGTANFPYLVTPEQAISNEVLKNKNGVFTATDNWAIDQIEALAKT
ASVSLVFVNADSGEGYINVDGNLGDRRNLTLWRNGDNVIKAAASN
CNNTIVIIHSVGPVLVNEWYDNPNVTAILWGGLPGQESGNSLADV
LYGRVNPGAKSPFTWGKTREAYQDYLYTEPNNGNGAPQEDFVEGV
FIDYRGFDKRNETPIYEFGYGLSYTTFNYSNLQVEVLSAPAYEPA
SGETEAAPTFGEVGNASDYLYPDGLQRITKFIYPWLNSTDLEASS
GDASYGQDASDYLPEGATDGSAQPILPAGGGAGGNPRLYDELIRV
TVTIKNTGKVAGDEVPQLVSRQQIRTKSDQANESQYVSLGGPNEP
KIVLRQFERITLQPSEETQWSTTLTRRDLANWNVETQDWEITSYP
KMVFVGSSSRKLPLRASLPTVH*
SEQ 5:
Polynucleotide sequence of the P_{gap} promoter +
gap ribosomal binding site (Zymomonas mobilis)
TCGACAATTTTACGCGTTTCGATCGAAGCAGGGACGACAATTGGC
TGGGAACGGTATACTGGAATAAATGGTCTTCGTTATGGTATTGAT
GTTTTTGGTGCATCGGCCCCGGCGAATGATCTATATGCTCATTTC
GGCTTGACCGCAGTCGGCATCACGAACAAGGTGTTGGCCGCGATC
GCCGGTAAGTCGGCACGTTAAAAAATAGCTATGGAATATAGTAGC
TACTTAATAAGTTAGGAGAATAAAC
SEQ 6:
Polynucleotide sequence of the Ppdc promoter +
pdc ribosomal binding site (Zymomonas
mobilis)
ATAATCACTTAATCCAGAAACGGGCGTTTAGCTTTGTCCATCATG
GTTGTTTATCGCTCATGATCGCGGCATGTTCTGATATTTTTCCTC
TAAAAAAGATAAAAAGTCTTTTCGCTTCGGCAGAAGAGGTTCATC
ATGAACAAAAATTCGGCATTTTTAAAAATGCCTATAGCTAAATCC
GGAACGACACTTTAGAGGTTTCTGGGTCATCCTGATTCAGACATA
GTGTTTTGAATATATGGAGTAAGCA
SEQ 7:
Polynucleotide sequence of the P_{tuf} promoter +
tuf ribosomal binding site Zymomonas
mobilis)
GCGATGGTGCCGCTGGCTAACATGTTCGGCTATGTGAACCAGCTC
CGTTCCTTCACCCAGGGACGCGCCCAGTATTCGATGCAATTTTCG
CATTATGACGAAGTCCCGGCTAACGTCGCGGATGAGTTAAAATCG
AAGATGGCTTAATTAAATACTGGCATAAACCGAAAAATGTCGTTA
TGAGCGCGCCGGAGAAGCGCGGCGCGCTCAATACAATAGTGATAA
AAGCGGTAACAAAAAGAGGTAACTA
SEQ 8:
Polynucleotide sequence of the Pₑₙₒ promoter +
eno ribosomal binding site (Zymomonas
mobilis)
AATCATTGGCAGCTGATGCTACATTCGCCACAGTTTTGTTTTCGG
CCATTGTCTATACTCCAGTTACTCAATACGTAACAATAATCAGTT
TATCCTAACTATAGAATCGCATGAGAAGCGATAACGTTTCACCAT
AAGCAATATATTCATTGCAACAGTGGAATTGCCTTATGCGTCAAG
GAAGGATAGATCATTGACGGACTGAGTTCAAAAAGAGACTCGTCT
AAAAGATTTTAAGAAAGGTTTCGAT
SEQ 9:
Polynucleotide sequence of the P_{eda} promoter +
eda ribosomal binding site (Zymomonas
mobilis)
ACTATAGAATATAAGTTATGTTCCATTCGCAGAATAGATATAGAT
CAGCCTCTATGGATATGCTATATATCGCCCATTCCATTTAAGAAT
AATAATAAACCATCATGCTGTTTATTTAATATTTTTATTACAGTG
AATTGAAGAAATATTTTCTTGATAAAAATTATTAAAAATCTATCA
CCGACGATCCGTCTCTATTTCAAGATAGATAATAATTTGTTTAAC
CTGTTGATTATGCGAGATAATTTTA
SEQ 10:
Polynucleotide sequence of the P_{zwf} promoter +
zwf ribosomal binding site (Zymomonas
mobilis)
GCAGCATTAAGTATCTTAGGTGGCTTGATTGTTGCTCGCTTCGTG
CCGGAAACCAAAGGTCGGAGCCTGGATGAAATCGAGGAGATGTGG
CGCTCCCAGAAGTAGTTAAACTTGCTTTGGCTGAATCCTTTTGTC
TTTTTTAGATAAGTCTTAACCAATTATACTTTTTGTTTACAACGA
TGGTATAAAGCGGGCGGACAGGCTAAAAACAGGCTAAAAGGATTC
GGCCTCTGTTTTAAGGACGAGAATA
SEQ 11:
Polynucleotide sequence of the P_{frk} promoter +
frk ribosomal binding site (Zymomonas
mobilis)
TTAAAAAATAACTTCATTTTACTTTAAATTTTCCAAGAAAATATT
TCGAAAATATTTTTGATATCTTTCTTAATTAAGAAAGAAAACTTA
GTTATAATCCTACCAGTTGGACGAATCGCAGACGGTCGATTTCGA
TTTATTCAAAAGGCCTTTTGGCACAGAAGAAAAATCGAGGTCATC
GTCATAATTTAAAGCGAATGGACAGCATATACCTCCGTATTACGG
GGGGATTTTGTGAGTGGTGAGAATA
SEQ 12:
Polynucleotide sequence of the P_{clcD1} promoter +
clcD1 ribosomal binding (Zymomonas
mobilis)
AGGCACCAACGCATCAAGAACATCAGCCGCCAATCGGCCTGCTTT
CCGCATTCCGGCAAAAGCGTTTTCATCGTAAAGTTTGATGGCATG
CGTACGAATAGCCGCATCACCTTCTTTGACATGGATATATTCGGT
CATAACTTCTGCTATAACCAATAAGCTTTCCCTTTGCGAGCCGCA
TTAACATATTCCGTTTTTGGATTCGGAATATCTGCCGCATCAGAG
GAAAGAGGAAAAGGACGGACTGAAA
SEQ 13:
Polynucleotide sequence of the P_{glms} promoter +
glms ribosomal binding (Zymomonas
mobilis)
GAACGGGATCAATATCGCTCCGATCCGATCCAGAAAATCAAACCC
TTCCATCAGAATACCTCGGATAAAGTTACCCAATTGGAAAAACTA
GCCGCCCTTAAAAATCAAGGCATCCTGACAGAAGAAGAATTTGAA
AAAGAAAAAAGACGTATTTTGGCCTTATAAAAGACGAAAATATTT
CTTACATTGCCCCCATCTCAAAGACAGTCCGCCTTTCAAAATAGA
TATCACAAAATCGGGAAACAGAATT
SEQ 14:
Zymomonas mobilis codon optimized polynucleotide
sequence of β-glucosidase 1 (bgl1)
ATGAGATTCACTTTAATCGAAGCGGTTGCTCTTACTGCCGTCTCT
TTAGCCAGCGCTGATGAATTGGCCTATTCCCCGCCGTATTATCCC
TCACCTTGGGCCAATGGCCAGGGTGACTGGGCAGAAGCATATCAG
CGCGCTGTTGATATAGTCTCTCAGATGACATTGGCTGAAAAGGTG
AATTTGACAACGGGGACTGGATGGGAATTGGAATTATGTGTTGGT
CAGACAGGGGGTGTTCCCCGATTGGGGATTCCGGGAATGTGTGCA
CAGGATAGCCCTTTGGGTGTTCGTGATTCCGATTATAATTCTGCG
TTTCCAGCCGGTGTCAATGTGGCCGCAACCTGGGACAAAAATCTG
GCTTATCTGCGCGGGCAAGCAATGGGTCAGGAGTTTAGTGATAAA
GGTGCTGATATCCAATTGGGTCCAGCTGCCGGCCCTCTGGGTCGG
AGTCCCGATGGCGGTCGTAATTGGGAAGGCTTTTCCCCGGATCCG
GCCTTATCCGGTGTGCTGTTTGCAGAAACAATTAAGGGTATTCAA
GATGCTGGTGTTGTTGCAACGGCTAAACATTATATTGCCTATGAG
CAGGAACATTTTCGTCAGGCGCCTGAAGCTCAAGGCTATGGATTC
AATATTACCGAGAGTGGAAGCGCGAACTTAGACGATAAAACGATG
CATGAGTTATATTTGTGGCCATTCGCGGATGCCATTCGTGCAGGT
GCCGGTGCTGTGATGTGCTCGTATAATCAGATTAATAACTCATAT
GGCTGTCAGAATAGCTACACACTGAATAAGCTGCTTAAGGCAGAA
TTAGGTTTCCAGGGCTTTGTCATGAGTGATTGGGCGGCTCATCAT
GCCGGTGTGTCAGGTGCTTTGGCAGGGTTGGACATGTCTATGCCG
GGGGACGTCGATTATGACTCAGGCACGTCTTATTGGGGTACCAAT
TTGACCATCTCAGTTCTGAACGGGACGGTTCCGCAATGGCGAGTT
GATGATATGGCTGTACGAATCATGGCCGCCTATTACAAAGTCGGC
CGTGACCGGCTGTGGACACCGCCAAACTTTAGCTCATGGACCAGA
GATGAATATGGCTTCAAATATTATTATGTCTCGGAAGGACCGTAT
GAAAAGGTCAATCAGTTTGTTAATGTGCAACGCAATCATAGCGAA
TTGATCCGCCGTATTGGAGCAGACAGCACGGTTCTGTTGAAAAAC
GATGGGGCTCTTCCGTTGACTGGAAAAGAACGCTTGGTCGCCCTT
ATCGGGGAAGATGCGGGTTCCAATCCTTATGGTGCCAACGGCTGC
AGTGATCGTGGGTGTGATAATGGAACATTGGCGATGGGCTGGGGA
AGTGGCACAGCCAATTTTCCCTACTTGGTGACCCCCGAACAGGCC
ATCTCCAATGAAGTTCTTAAGAATAAAAATGGCGTATTCACCGCA
ACCGATAATTGGGCTATTGATCAAATTGAGGCGTTAGCTAAAACC
GCCAGTGTATCTCTTGTCTTTGTCAATGCCGACTCTGGTGAAGGT
TATATTAATGTTGATGGGAACCTGGGTGATCGCCGTAACCTGACC
TTATGGAGAAATGGCGATAATGTCATCAAGGCTGCTGCTAGCAAC
TGCAACAATACGATCGTTATTATTCACTCTGTCGGCCCAGTCTTA
GTTAATGAATGGTATGACAATCCTAATGTTACCGCTATTTTATGG
GGTGGTTTACCTGGTCAGGAATCTGGCAATTCCCTTGCCGACGTG
TTATATGGCCGTGTCAACCCAGGTGCCAAATCGCCCTTCACCTGG
GGCAAAACTCGTGAAGCCTACCAAGATTATTTGTATACCGAGCCC
AATAACGGCAACGGAGCGCCACAGGAAGATTTTGTCGAAGGCGTC
TTTATTGATTATCGCGGATTTGATAAACGGAATGAAACACCTATC
TATGAATTTGGCTATGGTCTGAGCTATACCACCTTCAATTATTCG
AACCTTCAGGTGGAAGTTTTAAGCGCCCCTGCGTACGAACCTGCT
TCTGGCGAAACTGAGGCAGCGCCGACTTTTGGAGAAGTCGGAAAT
GCGTCGGATTATCTTTACCCCGATGGTCTGCAAAGAATCACCAAA
TTCATATATCCCTGGTTGAACTCAACCGATCTTGAGGCATCTTCT
GGGGATGCTAGCTATGGGCAGGATGCCTCAGACTATCTTCCAGAG
GGGGCCACCGATGGCTCTGCGCAACCGATTCTGCCTGCCGGTGGT
GGTGCTGGCGGCAATCCTCGCCTGTATGACGAACTTATCCGCGTT
ACGGTTACTATTAAAAATACCGGCAAAGTTGCAGGTGATGAAGTT
CCTCAACTGGTATCCAGACAACAAATTCGAACCAAATCGGATCAA
GCTAATGAATCGCAGTATGTTTCTCTTGGCGGCCCTAATGAACCC
AAAATCGTGCTGCGTCAATTCGAGCGGATCACGCTGCAACCGTCG
GAAGAAACGCAATGGAGCACGACTCTGACGCGCCGGGATCTTGCG
AATTGGAATGTTGAAACGCAGGATTGGGAAATTACGTCCTATCCG
AAGATGGTGTTTGTCGGAAGCTCCTCGCGGAAATTACCGCTTCGG
GCGTCTCTGCCTACAGTTCATTAA
SEQ 15:
Polynucleotide sequence of β-glucosidase 1
(bgl1) homologue (Aspergillus welwitschiae)
ATGAGGTTCACTTTGATCGAGGCGGTGGCTCTGACTGCCGTCTCG
CTGGCCAGCGCTGATGAATTGGCCTACTCCCCTCCGTATTACCCC
TCCCCTTGGGCCAATGGCCAGGGTGACTGGGCGGAAGCATACCAG
CGCGCTGTTGATATCGTCTCGCAGATGACATTGGCTGAGAAGGTC
AATTTGACTACGGGAACTGGATGGGAATTGGAATTATGTGTTGGT
CAGACTGGAGGTGTTCCCCGATTGGGAATTCCGGGAATGTGTGCA
CAGGATAGCCCTCTGGGTGTTCGTGACTCCGACTACAACTCTGCG
TTCCCCGCCGGTGTCAACGTGGCCGCAACCTGGGACAAGAATCTG
GCTTACCTTCGTGGCCAGGCTATGGGTCAGGAGTTTAGTGACAAG
GGTGCTGATATCCAATTGGGTCCAGCTGCCGGCCCTCTCGGTAGA
AGTCCCGACGGCGGTCGTAACTGGGAGGGCTTCTCCCCCGACCCG
GCCCTCAGTGGTGTGCTCTTTGCAGAGACAATCAAGGGTATTCAG
GATGCTGGTGTGGTTGCAACGGCTAAGCACTACATCGCCTACGAG
CAGGAGCATTTCCGTCAGGCGCCTGAAGCTCAAGGCTACGGATTC
AATATTACCGAGAGTGGAAGCGCGAACCTCGACGATAAGACTATG
CATGAGCTGTACCTCTGGCCCTTCGCGGATGCCATCCGTGCAGGT
GCCGGTGCTGTGATGTGCTCGTACAACCAGATCAACAACAGCTAT
GGCTGCCAGAACAGCTACACTCTGAACAAGCTGCTCAAGGCTGAG
CTGGGTTTCCAGGGCTTTGTCATGAGTGATTGGGCGGCTCACCAT
GCCGGTGTGAGTGGTGCTTTGGCGGGATTGGACATGTCTATGCCG
GGAGACGTCGATTACGACAGTGGCACGTCTTACTGGGGTACCAAC
TTGACCATCAGTGTGCTCAACGGGACGGTGCCCCAATGGCGTGTT
GATGACATGGCTGTCCGCATCATGGCCGCCTACTACAAGGTCGGC
CGTGACCGTCTGTGGACTCCTCCCAACTTCAGCTCATGGACCAGA
GATGAATACGGCTTCAAGTACTACTATGTCTCGGAGGGACCGTAT
GAGAAGGTCAACCAGTTCGTGAATGTGCAACGCAACCATAGCGAG
TTGATCCGCCGTATTGGAGCAGACAGCACGGTGCTCCTCAAGAAC
GATGGCGCTCTTCCCTTGACTGGAAAGGAGCGCTTGGTCGCCCTT
ATCGGAGAAGATGCGGGTTCCAATCCTTATGGTGCCAACGGCTGC
AGTGACCGTGGGTGCGACAATGGAACATTGGCGATGGGCTGGGGA
AGTGGCACTGCCAACTTTCCCTACTTGGTGACCCCCGAGCAGGCC
ATCTCGAACGAGGTGCTCAAGAACAAGAATGGCGTATTCACTGCG
ACCGATAACTGGGCTATTGATCAGATTGAGGCGCTTGCTAAGACC
GCCAGTGTCTCTCTTGTCTTTGTCAACGCCGACTCTGGTGAGGGT
TATATCAATGTCGACGGAAACCTGGGTGACCGCAGGAACCTGACC
CTGTGGAGGAACGGCGACAATGTGATCAAGGCTGCTGCTAGCAAC
TGCAACAACACGATCGTTATTATTCACTCTGTCGGCCCAGTCTTG
GTTAACGAGTGGTACGACAACCCCAATGTTACCGCTATTCTCTGG
GGTGGTCTTCCCGGTCAGGAGTCTGGCAACTCCCTCGCCGACGTG
CTCTACGGCCGTGTCAACCCCGGTGCCAAGTCGCCCTTCACCTGG
GGCAAGACTCGTGAGGCCTACCAAGATTACTTGTACACCGAGCCC
AACAACGGCAACGGAGCGCCCCAGGAAGACTTCGTCGAGGGCGTC
TTCATTGACTACCGCGGATTTGACAAGCGCAACGAGACTCCTATC
TATGAGTTCGGCTATGGTCTGAGCTACACCACCTTCAACTACTCG
AACCTTCAGGTGGAGGTTCTGAGCGCCCCTGCGTACGAGCCTGCT
TCGGGCGAGACTGAGGCAGCGCCGACTTTCGGAGAGGTCGGAAAT
GCGTCGGATTACCTCTACCCCGATGGACTGCAGAGAATCACCAAG
TTCATCTACCCCTGGCTCAACAGTACCGATCTTGAGGCGTCTTCT
GGGGATGCTAGCTATGGGCAGGATGCCTCAGACTATCTTCCCGAG
GGAGCCACCGATGGCTCTGCGCAACCGATCCTGCCTGCCGGTGGT
GGTGCTGGCGGCAACCCTCGCCTGTACGACGAGCTCATCCGCGTG
TCGGTGACTATCAAGAACACCGGCAAGGTTGCGGGTGATGAAGTT
CCTCAACTGTATGTTTCTCTTGGCGGCCCTAACGAACCCAAGATC
GTGCTGCGTCAATTCGAGCGTATCACGCTGCAGCCGTCGGAAGAG
ACGCAGTGGAGCACGACTCTGACGCGCCGTGACCTTGCGAACTGG
AATGTTGAGACGCAGGACTGGGAGATTACGTCTTATCCCAAGATG
GTGTTTGTCGGAAGCTCCTCGCGGAAGCTGCCGCTCCGGGCGTCT
CTGCCTACTGTTCAC
SEQ 16:
Polynucleotide sequence of β-glucosidase 1
(bgl1) homologue (Aspergillus luchuensis)
ATGAGGTTCACTTTGATTGAGGCGGTGGCTCTCACTGCTGTCTCG
CTGGCCAGCGCTGATGAATTGGCTTACTCCCCACCGTATTACCCA
TCCCCTTGGGCCAATGGCCAGGGCGACTGGGCGCAGGCATACCAG
CGCGCTGTTGATATTGTCTCGCAGATGACATTGGCTGAGAAGGTC
AATCTGACCACAGGAACTGGATGGGAATTGGAGCTATGTGTTGGT
CAGACTGGCGGGGTTCCCCGATTGGGAGTTCCGGGAATGTGTTTA
CAGGATAGCCCTCTGGGCGTTCGCGACTCCGACTACAACTCTGCT
TTCCCTTCCGGTATGAACGTGGCTGCAACCTGGGACAAGAATCTG
GCATACCTCCGCGGCAAGGCTATGGGTCAGGAATTTAGTGACAAG
GGTGCCGATATCCAATTGGGTCCAGCTGCCGGCCCTCTCGGTAGA
AGTCCCGACGGTGGTCGTAACTGGGAGGGCTTCTCCCCCGACCCG
GCCCTAAGTGGTGTGCTCTTTGCAGAGACCATCAAGGGTATCCAA
GATGCTGGTGTGGTCGCGACGGCTAAGCACTACATTGCCTACGAG
CAAGAGCATTTCCGTCAGGCGCCTGAAGCCCAAGGTTATGGATTT
AACATTTCCGAGAGTGGAAGCGCGAACCTCGACGATAAGACTATG
CACGAGCTGTACCTCTGGCCCTTCGCGGATGCCATCCGTGCGGGT
GCTGGCGCTGTGATGTGCTCCTACAACCAGATCAACAACAGCTAT
GGCTGCCAGAACAGCTACACTCTGAACAAGCTGCTCAAGGCCGAG
CTGGGTTTCCAGGGCTTTGTCATGAGTGATTGGGCGGCTCACCAT
GCTGGTGTGAGTGGTGCTTTGGCAGGATTGGATATGTCTATGCCA
GGAGACGTCGACTACGACAGTGGTACGTCTTACTGGGGTACAAAC
CTGACCGTTAGCGTGCTCAACGGAACGGTGCCCCAATGGCGTGTT
GATGACATGGCTGTCCGCATCATGGCCGCCTACTACAAGGTCGGC
CGTGACCGTCTGTGGACTCCTCCCAACTTCAGCTCATGGACCAGA
GATGAATACGGCTACAAGTACTACTATGTGTCGGAGGGACCGTAC
GAGAAGGTCAACCACTACGTGAACGTGCAACGCAACCACAGCGAA
CTGATCCGCCGCATTGGAGCGGACAGCACGGTGCTCCTCAAGAAC
GACGGCGCTCTGCCTTTGACTGGTAAGGAGCGCCTGGTCGCGCTT
ATCGGAGAAGATGCGGGCTCCAACCCTTATGGTGCCAACGGCTGC
AGTGACCGTGGATGCGACAATGGAACATTGGCGATGGGCTGGGGA
AGTGGTACTGCCAACTTCCCATACCTGGTGACCCCCGAGCAGGCC
ATCTCAAACGAGGTGCTCAAGAACAAGAATGGTGTATTCACCGCC
ACCGATAACTGGGCTATCGATCAGATTGAGGCGCTTGCTAAGACC
GCCAGTGTCTCTCTTGTCTTTGTCAACGCCGACTCTGGCGAGGGT
TACATCAATGTCGACGGAAACCTGGGTGACCGCAGGAACCTGACC
CTGTGGAGGAACGGCGATAATGTGATCAAGGCTGCTGCTAGCAAC
TGCAACAACACCATTGTTATCATTCACTCTGTCGGCCCAGTCTTG
GTTAACGAATGGTACGACAACCCCAATGTTACCGCTATTCTCTGG
GGTGGTCTGCCCGGTCAGGAGTCTGGCAACTCTCTTGCCGACGTC
CTCTATGGCCGTGTCAACCCCGGTGCCAAGTCGCCCTTTACCTGG
GGCAAGACTCGTGAGGCCTACCAAGATTACTTGGTCACCGAGCCC
AACAACGGCAATGGAGCCCCCCAGGAAGACTTCGTCGAGGGCGTC
TTCATTGACTACCGCGGATTCGACAAGCGCAACGAGACCCCGATC
TACGAGTTCGGCTATGGTCTGAGCTACACCACTTTCAACTACTCG
AACCTTGAGGTGCAGGTTCTGAGCGCCCCCGCGTACGAGCCTGCT
TCGGGTGAGACTGAGGCAGCGCCAACTTTTGGAGAGGTTGGAAAT
GCGTCGAATTACCTCTACCCCGACGGACTGCAGAAAATCACCAAG
TTCATCTACCCCTGGCTCAACAGTACCGATCTCGAGGCATCTTCT
GGGGATGCTAGCTACGGACAGGACTCCTCGGACTATCTTCCCGAG
GGAGCCACCGATGGCTCTGCGCAACCGATCCTGCCTGCTGGTGGC
GGTCCTGGCGGCAACCCTCGCCTGTACGACGAGCTCATCCGCGTG
TCGGTGACCATCAAGAACACCGGCAAGGTTGCTGGTGATGAAGTT
CCCCAACTGTATGTTTCCCTTGGCGGCCCCAACGAGCCCAAGATC
GTGCTGCGTCAATTCGAGCGCATCACGCTGCAGCCGTCAGAGGAG
ACGAAGTGGAGCACGACTCTGACGCGCCGTGACCTTGCAAACTGG
AATGTTGAGAAGCAGGACTGGGAGATTACGTCGTATCCCAAGATG
GTGTTTGTCGGAAGCTCCTCGCGGAAGCTGCCGCTCCGGGCGTCT
CTGCCTACTGTTCACTAA
SEQ 17:
Polynucleotide sequence of β-glucosidase 1
(bgl1) homologue (Aspergillus sclerotioniger)
ATGAGGTTCAGTTGGATCGAGGTGGCGGCTCTGACAGCCGCCTCG
GTGGTCAGCGCTGATGAATTGGCGTACTCTCCCCCTTTCTACCCT
TCTCCCTGGGCCAATGGCAAGGGTGACTGGACAGACGCGTACAAG
CGTGCGGTCGACATTGTTTCGCAGATGACACTGGCCGAAAAGGTC
AATCTGACAACGGGAACTGGATGGGAATTGGAAAAGTGTGTCGGT
CAGACCGGCGGTGTTCCTCGATTGGGAATACCGGGCATGTGTGGT
CAGGATAGTCCTCTGGGTGTTCGTGACTCTGACTACAACTCGGCT
TTCCCTGCTGGCATCAACATTGCCGCTTCCTGGGACAAGGACCTC
GCGTACCTCCGTGGCAAGGCCATGGGCCAGGAGTTCAGTGACAAG
GGTGCTGATATTCAATTGGGCCCCGTTGCTGGTCCCCTCGGTAGA
AGTCCCGACGGCGGTCGTAACTGGGAGGGGTTTGCTCCAGACCCG
GCCCTGACTGGTGTGCTCTTTGCAGAGACCATCAAGGGTATCCAG
GATGCTGGCGTGGTTGCGACGGCTAAGCACTTCATCGGCTATGAG
CAAGAGCATTTCCGCCAGGCACCTGAGGCTCAAGGATATGGGTAC
AACATCACCGAGAGTGGCAGCGCGAACATCGACGATAAGACCATG
CACGAGCTGTACCTCTGGCCCTTTGCGGATGCTATCCGCGCAGGT
GTTGGCGCGGTCATGTGCTCCTACAACCAGATCAACAACAGCTAC
GGTTGCCAGAACAGCTATGCTCTGAACAAGCTCCTTAAGTCTGAA
CTGGAATTCCAGGGCTTCGTGATGAGCGACTGGGAGGCCCACCAC
AGTGGAGTCGGTGCTGCTTTGGCCGGTCTGGACATGTCTATGCCC
GGAGATGTCACCTACGACAGTGGCACGTCTTACTGGGGTGCCAAC
CTGACCATCAGTGTCCTGAACGGCACGGTTCCCCAGTGGCGTGTT
GATGATATGGCTGTCCGTATCATGGCCGCCTACTACAAGGTCGGT
CGGGATCGTCTGTGGACGCCTCCCAACTTCAGTTCATGGACCCGC
GATGAATACGGCTACAAGTACTACTATTCCTCCGAGGGACCGTAT
GAGAAGGTCAACCAGTTTGTCAACGTGCAGCGCAACCATAGCGGG
TTGATCCGCCGTATTGGAGCTGACAGCACGGTTCTTTTGAAGAAC
GAGAACGTTCTGCCCCTGACTGGCAAGGAGCGCCTGGTCGGTCTT
ATCGGAGAAGACGCGGGCTCCAACGCTTACGGCGCCAACGGCTGC
AGTGACCGTGGATGCGACAACGGTACTCTGGCTATGGGCTGGGGC
AGTGGTACCTCGAACTACCCTTACTTGATCACTCCCGAGCAGGCG
ATCCAGGCTGAGGTGATCAAGAACAAGGGCAATGTATTTGCCGTG
ACCGACAACTGGGCCATTGACCAGATTGAGGCACTCGCCAAGCAA
TCCAGTGTCTCTCTTGTCTTTGTCAACGCCGACTCTGGTGAAGGT
TACATCGATGTCGATGGCAACATGGGTGACCGCAACAACATTACG
CTCTGGAGGAACGGTGACAACGTGGTCAAGGCCGCTGCCAACAAC
TGCAACAACACCATTGTCATCATCCACTCCGTCGGCCCGGTCCTC
GTCACCGAGTGGTACGACCACCCCAATGTCACCGGCATACTGTGG
GCTGGCTTGCCCGGCCAGGAGTCTGGCAACTCGCTCGCCGATGTG
CTCTACGGCCGTGTCAACCCGGGTGCCAAGTCGCCCTTCACCTGG
GGTAAGACCAGGGAGTCCTACCGCGACTACCTGATCACCGAGCCC
AACAATGGCGATGGAGCCCCACAGGAAGACTTCACCGAGGGCATC
TTCATCGACTACCGCGGGTTCGACAAGCGCAATGAGACCCCAATC
TACGAGTTCGGCTATGGCCTGAGCTACACCACCTTCAACTACTCG
AATTTGAACGTGCAGGTCCTGAATGCCTCGTCGTACACTCCTTCT
TCTGGCGAGACTGAGGCTGCCCCGACTTTTGGAGAGATCGGCAAC
GCGTCGGACTACCTCTACCCTAGCGGACTGAAGAAGATTACCGAC
TTCATCTACCCCTGGCTTAACAGCACGGACCTCCTGGAAGCTTCC
GGCGACGCCAGCTACGGTCTGAACGCGTCGGAGTATATCCCCGAG
GGAGGCACCGATGGCTCTGCCCAGCCGATCCTGGCTGCTGGTGGT
GGCCCTGGCGGTAACCCCGGTCTGTATGACGAGCTGATTCGCGTT
TCGGCGACCATCAAGAACACCGGCAAGCTTGCGGGTGACGAGGTT
CCTCAACTGTACGTTTCGCTTGGAGGTCCCGATGACGCCAAAATT
GTGTTGCGCAAGTTCGACCGCATCTCTCTCAAGCCATCGCAGGAG
GTTGAGTGGAGCACGACTCTGACACGACGCGACCTGGCAAACTGG
GATGTTGCGGCGCAGGACTGGACCATCACGTCTTATCCCAAGACG
GTGTATGTCGGTAGCTCTTCGCGGAAGCTGCCGCTCCGCGCGTCA
CTGCCTACTGTCCAATAG
SEQ 18:
Polynucleotide sequence of β-glucosidase 1
(bgl1) homologue (Aspergillus tanneri)
ATGAGGTTTGGTTGGTTTGAGGCGGCGGTCGTGACCGCTGCCTCA
GTGGTCAGTGCCCAGGATGATCTTGCTTTCTCTCCTCCGTACTAT
CCTTCCCCGTGGGCCAATGGCCAGGGAGAATGGGCCGACGCGTAT
GAACGTGCCGTCGACATCGTCTCCCAGATGACGTTGGCGGAGAAG
GTTAACCTCACTACTGGAACAGGGTGGATGTTGGATAAATGTGTG
GGTCAGACAGGAAGTGTTCCCAGACTCGGACTATTAAGTCTCTGC
TTGCAAGACAGTCCCTTGGGTATTCGGTTTTCGGATTACAATTCG
GCATTCCCCGCAGGTGTTAATGTCGCCGCAACATGGGACAAGCAA
CTGGCGTACCTCCGCGGTAGGGCAATGGGTGAGGAATTCAGCGAC
AAGGGAATCGACGTTCAATTAGGCCCTGCTGCTGGGCCTCTCGGC
AGACACCCCGATGGTGGTCGGAACTGGGAAGGTTTCTCCCCTGAC
CCTGCCCTTACCGGTGTACTTTTCGCAGAAACCATAAAAGGCATC
CAGGATGCTGGTGTGATCGCGACGGCCAAGCATTACATTTTGAAT
GAACAGGAGCATTTCCGTCAAGTTCCCGAAGCCCTTGCGGCTGGA
TTCAATATTTCGGACTCCCTGAGCTCCAATCTAGACGACAAAACC
ATGCATGAGCTGTACCTCTGGCCCTTCGCAGATGCAGTGCGCGCG
GGTGTGGGTGCTGTGATGTGCTCCTACACTCAGATCAATAACAGT
TACGGCTGCCAGAATAGCGAAACTCTGAATAAACTTCTCAAGGCG
GAGCTTGGCTTCCAGGGTTTTGTCATGTCGGACTGGAGTGCGCAC
CACAGCGGTGTCGGCTCTGCCTTGGCGGGGCTGGATATGTCAATG
CCCGGGGATATCGCCTTCAATGACGGCAATTCGTACTTCGGGGCA
AACTTGACGATTGGCGTCCTCAACGGAACCATCCCTCAGTGGAGA
GTGGACGACATGGCTGTCCGCATCATGGCTGCTTACTATAAGGTT
GGCCGGGATCGCCACCACGCGCCTCCCAACTTCAGCTCATGGACC
AGGGACGAATACGGCTACGAGCACGCTGCAGTTTCGGAGGGCGCG
TATGAAAGGGTGAACCAATTTGTCGACGTGCAGCGGGACCATGCG
GAAATCATCCGTCGTGTGGGTGCCGAGAGCATTGTTCTCTTGAAG
AACGAGAACGCTCTGCCGTTGAGTGGGAAGGAGAAGAAGGTCGTT
ATCCTCGGAGAAGATGCGGGATCCAACCCTTGGGGTGCCAATGGC
TGCGAAAACCGTGGATGTGACAATGGGACCCTTGCCATGGCTTGG
GGCAGTGGCACTACAGAGTTCCCCTACCTCGTCACTCCCGAACAG
GCGATCCAGAACGAGGTCCTCCGAGGTCGTGGTAATGTATTTAGT
GTCACCGACAACGGGGCTCTGAGTCAGATGGCGGCGCTTGCCTCT
CAATCTAGTGTCGCTCTTGTCTTTGTCAATGCCGACTCGGGTGAG
GGATTCATCACCGTGGATGGAAACGAGGGAGATCGCAAGAACCTC
ACTCTCTGGAAGAACGGAGAGAACGTGATCAAAACCGCTGCCCAA
AACTGCAACAACACCGTCGTGATCATCCACTCCGTCGGAGCCGTT
CTGGTTGACGAATGGTATGACCACCCCAATATTACCGGCATTCTT
TGGGCGGGTCTGCCTGGTCAGGAGTCCGGGAACTCCCTCGCCGAC
GTGCTGTACGGCCGTGTCAACCCTGGCGGTAAGACCCCCTTCACC
TGGGGTCGAACCCGCGAATCGTATGGGGCACCCCTGGTCACCGAT
GCGAACAACGGCCACGGTGCGCCCCAGTCGGACTTCGAAGAAGGG
GTGTTTATTGACTACCGTCATTTCGACAAGTTCAACGAGACTCCC
ATCTACGAGTTTGGCTACGGCCTTAGTTACACCACTTTCGGCTAC
TCGGGACTGCAAATTGAGCCCCTGAACGCGCCCAAGTACGTCCCC
AACTCCGGGAAGACCGAAGCGGCGCCAACTTTCGGCGATGTTGCA
GAGGTCTCAGACTATGTGTATCCAAAGGGACTGCGGAGAATCCGT
GAGTTCATCTACCCCTGGCTGAATTCCACCAATCTGAAGAAGTCC
TCCGGCGATGCCACTTATGGAGGGGAGGACTCGACGTACATCCCC
GATGACGCCACCGATGGTTCTCCTCAGGACCTTCTGCCTGCCAGC
GGTGGTCCCGGAGGCAATCCAGGTCTCTACCAGGATCTGGTGCGG
GTGTCGGTTACGATCACGAACACGGGCAACGTGGCCGGCTACGAC
GTGCCTCAGCTTTATGTTTCCCTTGGTGGCCCCAACGAGCCGAAG
GTGGTACTCCGCAAATTCGATCGATTTAAGCTGGATCCGTCGCAG
CAGGTGGTGTGGTCGACCACGCTGAATCGTCGCGACCTGTCCAAC
TGGGACGTGACGGGGCAGGACTGGGTCATCACCGAGTATCCCAAG
AAGGTCTTCGTTGGTAGCTCGTCCCGGAAATTGCCGCTACATGCT
TCCTTGCCGGAGATGGAGTAA
SEQ 19:
Polynucleotide sequence of β-glucosidase 1
(bgl1) homologue (Aspergillus glaucus)
ATGAAGCTCGGCTGGTTGGAGTTTGTTGCCGTCACGGCCTCGGTG
GCCCAGGCTAAGGACCTCGCGTACTCTCCCCCCTACTATCCCTCG
CCATGGGCCGATGGTCAACCCGCCGAATGGTCCAACGCGTACAAA
CGCGCCGTCGACATCGTCTCCAACATGACTTTGGCGGAAAAGGTC
AACTTGACCACTGGTACTGGCTGGCAATTGGAAGAATGTGTTGGT
CAGACTGGTAGTGTTCCACGACTTGGTATCTGGGGTATCTGTTTG
CAGGACTCGCCTCTTGGTATCCGTTATGGCGATCACAGCTCTGGC
TTCCCCGCTGGTCTCAACGTCGCCGCGACCTGGGACCGCAAGCTC
GCCTACCTCCGTGGTGAGGCTATGGGTCAGGAATTCAGCGACAAA
GGAATTGACGTCCAATTGGGTCCCGTTGCTGGCCCTATCGGCAGA
TCTCCCGACGGTGGTCGCAACTGGGAGGGTTTCGCCCCCGACCCG
GTTCTGACCGGTGTGCTCATGGCCGAGACTATCAAGGGTATTCAG
GATGCCGGTGTCATCGCTACTGCTAAGCACTACATCGGCAACGAG
CAGGAGCATTTCCGTCAGGTTTCCGAGGCTCTCGACTATGGTTAC
AACATCACTGAGACTGCCAGCTCCAACATCGACGACAAGACCATG
CACGAGCTCTACTTGTGGCCTTTCGCCGATGCTGTTCGTGCTGGT
GTCGGTTCCGTCATGTGCTCGTACAACCAGATCAACAACAGCTAT
GGTTGCCAGAACAGTCATTTGCTGAACAAGCTGCTCAAGCACGAG
CTGGGCTTCCAGGGCTTCGTTATGACCGACTGGGGTGCTCACCAT
AGCGGTGTCGCCTCCACCCTTGCTGGTACTGACATGTCGATGCCC
GGTGATATCTCGTTTGACGATGGTATGTCGTACTTCGGTCCCAAC
CTGACTGTTGCTGTTCTCAACGGTACTGTTCCCGAGTGGCGTGTC
GACGACATGGCCGTTCGTATCATGTCTGCTTTCTACAAGGTTGGC
CGTGACCGCCAGCGCACGCCTCCCAACTTCAGCTCCTGGACCAAC
GACGAGTACAGCTACGCCCACTCCGCCGTCCAGGAAGGCTGGAAG
CAGGTCAACCAGCACATCAATGTCCAGCGCAACCACTCCGAGATC
ATCCGTGAGGTTGGCGAAGCCAGCACTGTTCTCTTGAAGAACAAG
GGTGCTCTTCCTCTGACTGGCGACGAGGGCTCCGTTGGTATTCTA
GGCGAAGATGCCGGGTCCAACGCGTATGGTGCCAACGGCTGCGAG
GACCGTGGATGCGACAATGGTACCCTTGCGATGGCCTGGGGTAGC
GGTTCCGCCGAATTCCCTTACCTGGTTACTCCCGAGCAGGCTATC
CAGAACGAGATTCTCAACCGGGACGTCAAGCACCCCGTCTTCGCC
GTCACTGACAACTGGGCTTTGGATCAGATGGCCTCCATTGCCTCT
CAATCTGATGTTTCTCTTGTCTTCGTCAACGCCGATGCTGGTGAG
GGTTTCCTCGTCGTGGATGGTAATGAGGGTGACCGCAATAACATC
ACCCTCTGGAAGAACGGTGAGAATGTCATCAAGACTGTCAGCGAG
AACTGCAACAACACCATTGTGATCATGCACACCGTCGGACCCGTC
CTCATCGACCAGTGGTACGACAACCCCAACATCACCGCCATTGTC
TGGGCTGGTCTGCCTGGCCAGGAATCCGGAAACGCCATTGCCAAC
GTTCTCTACGGCCGTGTCAACCCTGGTGGCAAGAGCCCCTTCACC
TGGGGTAAGAGCCGCGAGGCATACGGTGCCCCGCTCCTCACCGAG
ACAAACAACGGCATCGGCGCTCCTCAGGTCGACTTTACTGAGGGT
CAGTTCATCGACTACCGGCGCTTCGACAAGTACAACGAGACCCCT
ATCTACGAGTTTGGCTACGGTCTGAGTTATACCACCTTCAAGTAC
TCCAACCTCCACGTCCAGGCCCTGAACGCCTCCAAGTACGTTCCC
ACCACTGGCAAGACCAGTGCCGCGCCCAAGCTTGGTGAGGTTGGC
AAGGCTTCGAACTACGTCTTCCCCAATGGATTCGAGCGCACCACC
AAGTTCATCTACCCCTGGTTGAACTCGACCGATCTGAAGAAGTCC
GCCAACGACCCCGAGTACGGCCTCGAGATCTCAAAGTACATCCCC
GAGAACGCTCAGGACGAATCCTCCCAGGCCCGTTTGCCAGCCAGC
GGCGGCCAAGGAGGCAACACCGGTCTCTACGACGAACTCTTCCGC
GTCTCCGCCACGATCAAGAACACCGGCAAGGTCGCAGGTGACGAA
GTTCCCCAGCTGTACGTCTCTCTCGGCGGCCCCAACGAGCCCAAG
GTTGTCCTGCGCAACTTTGACCGCATCGCCCTCCAGCCTGGTCAG
GAGGTCGTGTGGTCGAAGACTCTTACCCGTCGTGACCTTTCCAAC
TGGGACGTTGCCGCTCAGGACTGGGCTATCACGCAGCATACCAAG
AAGGTGTTTGTTGGGAGCTCGTCGCGCAAGCTGCCTTTGCGGGCT
TCGTTACCTCGCGTGCAGTAG
SEQ 20:
Polynucleotide sequence of β-glucosidase 1
(bgl1) homologue (Penicillium argentinense)
ATGAAGCTCGGGTTGCTCGAAGCCGCCGTCTTGACGGCCGCCTCG
GCCGCCACTGCGTCGGACCTGGCATACTCGCCCCCGTACTATCCG
TCCCCGTGGATGACCGGCGAGGGCGACTGGGCCGAGGCCTACCGC
CGCGCCGTCGACTTCGTCTCCAACCTCACACTGGCTGAAAAGGTC
AACTTGACCACTGGTGCGGGCTGGGAGCAAGAACGCTGCGTCGGG
GAGACGGGTGGTATTCCCCGACTGGGAATGTGGGGCATGTGCTTG
CAAGACTCGCCCCTGGGCATCCGCGACAGTGACTACAACTCCGGT
TTCGCCGCGGGCGTCAATGTTGCCGCCACATGGGACAAGAGACTC
GCCTACCAGCGTGGTCTGGCGATGGGCGAGGAGCACCGCGACAAG
GGTGTCGATGTGCAGCTCGGTCCTGTGGCCGGCCCACTGGGACGA
AGCCCCGATGGCGGCCGTGGCTGGGAAGGGTTCTCGCCTGATCCT
GTCCTCACCGGTGTTATGATGGCGGAGACTATCAAGGGTATTCAG
GATGCTGGTGTCATTGCTTGCGCCAAGCACTATATCGGAAACGAG
CAGGAACACTTCCGCCAGTCGGGCGAGGCTCAGGGCTATGGGTAC
AACATCACTGAGAGTGTGAGCTCCAACATCGACGACAAGACTATG
CACGAACTGTACCTCTGGCCCTTTGTCGACTCTATCCGCGCCGGT
GTCGGCTCTGTCATGTGCTCGTACAATCAGATCAACAACAGCTAT
GGATGCCAGAACAGCGAGACCTTGAACAGGTTGCTCAAGGCTGAG
CTGGGTTTCCAGGGCTTCGTCATGTCGGATTGGGGTGCGCACCAT
AGCGGTGTCAGCGCTACCCTTGCTGGTCTGGATATGTCCATGCCC
GGTGATGTCATCCTCGGTAGCCCGTACTCCTTCTGGGGCACGAAC
CTGACCATCTCCGTGCTGAACGGTACCGTCCCCGAATGGCGTATC
GACGATATGGCCGTCCGCATCATGTCGGCCTACTACAAGGTCGGC
CGTGACCGTGTCCGTGTCCCTCCCAACTTCAGCTCCTGGACTCGT
GATGAGTATGGCTTTGAGCACTTCATGGTCAGCGAGAACTACATC
AAGCTCAACGAGCGCGTCAATGTCCAGCGCGACCACGCCGCGGGT
ATCCGCAAGCTTGGCTCTGACAGCACCGTTCTGCTGAAGAACAAG
GGTGCTCTGCCCTTGACTCACAATGAGAAGTTCGTTGGTATTCTG
GGTGAGGATGCTGGTTCCAACCCTGCTGGCGCTAATGGCTGTGCG
GACCGTGGTTGCGATGACGGCACCCTTGCTATGGGCTGGGGTAGT
GGTACTGCTAACTTCCCTTACCTGATTACCCCCGAGCAAGCCATT
CAGAACGAGGTTCTGAACTATGGCAATGGCCAGACAAATGTGTTT
GCCGTGACCAACAACACCAACACGGAGCAGATTGCTGCCATTGCT
GCGCAGTCGAGCGTCGCTCTCGTCTTCGTAAACGCCGACTCCGGC
GAAGGCTTCATCAATGTCGACGGCAACGAGGGTGACCGCAAGAAC
CTAACCCTCTGGAAGAACGGCGAACACCTCATCAAAACAGCTGCG
GCAAACTGCAACAACACCGTCGTCATCATGCACACCCCCGGCGCC
GTCCTAATCAGCGACTGGTACGAGCACGACAACATCACCGCCATC
CTCTGGGCCGGTCTACCGGGCCAAGAAAGCGGCCGCAGTATCACA
GACATTCTCTACGGGCGCGTGAACCCAGGCGGCAAAACACCCTTC
ACCTGGGGAAAGACTCGCAAAGACTACGGCGCACCGCTCCTCACA
CAACCCAACAACGGCCATGGTGCGCCGCAGCAAGACTTCACAGAG
GGAGTCTTCATCGACTACCGCCGCTTCGACAAGGAGCAGGAAGAG
CCTGTCTACGAATTCGGGTACGGACTCAGCTACACGAATTTCGAA
TTCAGCGATCTGCACATCAAGTCCCTTAACGCAGACAAGTATGTC
CCGACAACGGGGACAACCAAGCCTGCCCCCGTCTTCGGCAAAATC
GGCCAGGCATCCGACTATCTCTTCCCGAAGGGCATCCACCGTGTC
ACCCAATACCTCTACCCCTACCTGAACAGCACAAACCTCAAGTCC
TCCTCCGGCGACCCCTACTACGGCGCTAAGACAGAGGAGTACATC
CCCGCCGGCGCAACCAACGGCTCCGCGCAAACCCGCCTCCCATCC
AGCGGTGCCAACGGCGGTAATGCGGGTCTCTTCGAGGATCTGTAC
CAGGTTACCGTGACAATTACCAACACCGGCTCTGTGCAGGGCGAC
GAAGTTCCCCAGTTGTACGTTAGTCTGGGCGGGGAGAATAACCCC
GTTAAGGTCCTGCGTGCGTTTGACAGGATCACCATTGCGCCTGGC
CAGAAGGCACAGTGGACTACTACTCTTACCCGCCGCGATTTGTCG
AGTTGGGATGTTGCAAAACAGAATTGGGTAGTTACTCCTGCGCAG
AAGAAGGTGTATGTTGGAAATTCGTCGCGGAGACTTCCGCTTGAG
TCGGAGTTGCCCGCTTCGAAATGA
SEQ 21:
Polynucleotide sequence of β-glucosidase 1
(bgl1) homologue (Aspergillus wentii)
ATGAGGTTCGGTTGGTTGGAGGTAGCCGCTCTTACGGCTGCCTCC
GTGGTCAGTGCCAAGGATGACCTGGCATTCTCCCCTCCCTTCTAC
CCCTCTCCGTGGGCAAACGGTGAGGGTGAATGGGCAGACTCGTAC
AAGCGCGCTGTCGAGTTTGTTTCGAACTTGACCTTGGCTGAGAAG
GTCAACCTTACAACTGGTTCTGGCTGGCAGCAAGAGAGATGTGTT
GGTGAGACGGGCGAAGTTTCTAGACTTGGATTCTGGGGTATTTGT
TTGCAGGATTCTCCCCTGGGTATTCGTTTTGGCGACCACTCCTCC
GGCTTCCCCTCCGGCCTCAACGTTGCTGCAACCTGGGACAAAAAG
CTTGCCTACCTCCGTGGTAAGGCAATGGGCGAGGAATTCCGTGAC
AAGGGCATTGATGTTCAACTGGGACCTGTCGCTGGTCCCCTTGGC
GCTTTCCCTGATGGTGGTCGAAACTGGGAGGGTTTCGCTCCCGAC
CCTGTGCTGACTGGTTTCCTGATGTCGGAGACTATCAAGGGTATC
CAAGATGCTGGTGTTATTGCTACTGCTAAGCATTACATCGGTAAT
GAGCAGGAGCATTTCCGCCAAAGCGGCGAGGCTAAGGGTTATGGT
TACAACATTACTGAAAGTTCGAGCTCAAACATTGATGACAAGACA
ATGCACGAGTTGTATCTCTGGCCTTTTGCTGATGCCGTTCGTGCT
GGTGTCGGCGCGTTCATGTGCTCATACAACCAGATCAACAACAGC
TATGGCTGCTCCAACAGCTACCTGATGAACAAACTCCTCAAGTCT
GAACTAGGCTTCCAAGGATTTGTCATGAGTGATTGGGGTGCACAC
CACAGCGGTGTTGGCGCTGCTTTGGCTGGTTTGGATATGTCCATG
CCTGGTGACACTGTCATGGGCGACCCCTACACCTTCTGGGGAACC
AACCTGACCATCTCGGTTCTCAACGGCACCGTCCCCGAGTGGCGT
GTGGATGACATGGCCGTCCGTATCATGGCTGCTTACTACAAGGTG
GGCCGCGATGAGGTTCGCACCCCTCCCAACTTCAGCTCGTGGACC
ACCGAAGAATTCGGATACGCGCATTATGCTGCTCAGGAAGGATAC
GAGAAGACCAACTGGAATGTCAATGTCCGCCGCAACCACGCCAAG
GTCATCCGCGAAATCGGTTCGGCCAGCACGGTTCTTCTGAAGAAC
AATGGCGTGCTCCCCCTGACCGGTGATGAGGACTATGTCGGAATT
CTGGGCGAGGACGCCGGAGCTAACCCCTATGGTGCCAACGGCTGT
GAAGACCGTGGCTGCGACAATGGTACTCTCGCTATGGCTTGGGGC
AGTGGTAGTGCGGAATTCCCTTACCTTGTGACTCCCGAGCAGGCC
ATCCAGAACGAGGTTCTCAAGGGCGAGGGCTCTGTGTTCGCTGTG
ACCGATAACTGGGCTCTCGAGCAGATGGCTTCTATTGCTTCGCAG
TCCTCTATCTCTCTCGTCTTTGTCAATGCCGACTCCGGAGAAGGT
TTCCTCAATGTGGACGGAAACATGGGTGACCGCAAGAACTTCACC
CTCTGGAAAAATGGCGAGAATGTGATCAAGACTGTCACTGAGAAC
TGCAACAACACCGTTGTGGTCATGCACACTGTTGGCCCGGTCCTG
ATCAAGGACTGGTATGACAACCCTAACATCACTGCCATTGTCTGG
GCTGGTTTGCCTGGCCAGGAAAGCGGAAACTCTCTCGCTGATGTG
CTTTACGGCCGTGTCAACCCTAGTGGCAAGAGCCCATTCACCTGG
GGCAAGACTCGCGAGGCTTATGGTCCTTCTCTGCTCACCACCCAG
AACAACGGCAATGACGCTCCCCAGCAAGATTTCACCCAGGGTGTC
TTCATTGACTACCGCCGATTTGACAAGTTCAACGAGACTCCCATC
TACGAGTTCGGATACGGTCTGAGTTACACCACCTTCAACTACTCC
AACCTCGAAGTTCGATCCCTGAATGCATCGCGGTACACCCCAACC
ACTGGCAAGACTGATGCCGCGCCTTCTCTGGGCGAGGCTGGCAAG
GCCTCGGACTTTTTGTTCCCCAAGGGACTGAACCGCATCATCGGC
TACATTTATCCGTGGTTGAACTCGACCGACCTGAAGTCGGCGTCC
GGAGAGAAGAACTATGGCATGAAGGCTTCTGAGTACATTCCCGAG
GGAGCCACCGATGGATCCGCCCAGGAGCTCCTGCCGGCCGGCGGT
GGCCCTGGTGGTAACCCTGGTCTGTATGAAGACCTCATCGAGGTT
TCTGCTACTATTACCAACACTGGCAAGGTTGCTGGTGACGAAGTC
CCACAGCTGTATGTCTCCCTTGGTGGTGCCGACGACCCCGTTCTT
GTCCTCCGTCAGTTCGACCGTCTCCACATCGAGGCTGGAAAGCAG
GCAGTGTGGAAGACGACTCTTACCCGCCGTGACCTCGCCAACTGG
GACGTTGCGGCGCAAGACTGGACCATCACGAAGAGCGCGAAGAAG
GTGTACGTGGGCAGCTCTTCGCGGAACCTGCCCCTCAAGGCCAAG
CTTCCCACCGTTCAGTAG
SEQ 22:
Polypeptide sequence of β-glucosidase 1 (BGL1)
homologue (Penicillium vulpinum)
MSTIIAPVSKELFEYLRTHAFPEICDSFLPHAFFYQQRFLCCLCE
ILSSIFTAMKFEWLTVAGLTAAANANPLAYSPPYYPSPWMTGAGE
WSDAYTRAVDFVSNLTLAEKVNLTTGAGWEQERCVGETGGIPRLG
MWGMCMQDSPLGVRLSDYTSGFPSGINVAATWDKRLAYQRGMAMG
EEHRDKGVDVQLGPVAGPLGKYPEGGRNWEGFSPDPVLTGVMMAE
TIKGMQDAGVIACAKHFIGNEQEHFRQSGEAQGYGYNISESVSSN
IDDKTMHELYLWPFVDSIRAGVGSIMCSYNQINNSYGCANSYALN
KLLKGELGFQGFVMSDWGAHHSGVSSTLAGLDMSMPGDTYLGSPS
SFWGANLTISVLNGTVPEWRIDDMAVRIMAAYYKVGRDRFRTPPN
FSSWTRDEYSFEHAAVSEGWAKVNERVNVQRDHAQIIRKIGSDST
VLLKNKGGALPLTHGEKFISILGEDAGSNAYGANGCGDRGCDNGT
LAMGWGSGTANFPYLITPEQAIQNEVLEYSVGKTSVFAVTDNWAL
TEMAALASQADVALVFVNADSGEGYINVDGNEGDRKNLTLWKNGE
EIIKTASQHCNNTIVVIHSTAAVLISDWYDNDNITAIVWAGLPGQ
ESGRSLVDVLYGRINPGGKTPFTWGKTRKDYGPPLLTVPNNGADA
PQDNFEDGVFIDYRRFDKDNTEPIYEFGYGLSYTNFSFSDLKVTP
LASSEYNEYKATTGKTKKAPVLGKAGKVSDNLFPEGIKPVRQYLY
PWLNSTDLRASSGDPAYGMDSKDYLPEGATDGSPQDLLPSSGASG
GNPDLFKNLYQVTATITNTGSVTGDEVPQLYVSLGGDDEPSKVLR
QFDRVTIAPGQTLQWTTTLTRRDVSNWDVASQNWVISDAPKKVYV
GNSSRKLPLSADLPPI
SEQ 23:
Polypeptide sequence of β-glucosidase 1 (BGL1)
homologue (Aspergillus terreus)
MKLSILEAAALTAASVVSAQVTFTLPVNLVLDGALTVRKQDDLAY
SPPYYPSPWADGHGEWSNAYKRAVDIVSQMTLTEKVNLTTGTGWE
LERCVGQTGSVPRLGIPSLCLQDSPLGIRMSDYNSAFPAGINVAA
TWDKTLAYQRGKAMGEEFSDKGIDVQLGPAAGPLGRSPDGGRNWE
GFSPDPALTGVLFAETIKGIQDAGVIATAKHYILNEQEHFRQVGE
AQGYGFNITETVSSNVDDKTMHELYLWPFADAVRAGVGAVMCSYN
QINNSYGCQNSLTLNKLLKAELGFQGFVMSDWSAHHSGVGAALAG
LDMSMPGDISFDSGTSFYGTNLTVGVLNGTIPQWRVDDMAVRIMA
AYYKVGRDRLWTPPNFSSWTRDEYGFAHFFPSEGAYERVNEFVNV
QRDHAQVIRRIGADSVVLLKNDGALPLTGQEKTVGILGEDAGSNP
KGANGCSDRGCDKGTLAMAWGSGTANFPYLVTPEQAIQNEVLKGR
GNVFAVTDNYDTQQIAAVASQSTVSLVFVNADAGEGFLNVDGNMG
DRKNLTLWQNGEEVIKTVTEHCNNTVVVIHSVGPVLIDEWYAHPN
VTGILWAGLPGQESGNAIADVLYGRVNPGGKTPFTWGKTRASYGD
YLLTEPNNGNGAPQDNFNEGVFIDYRRFDKYNETPIYEFGHGLSY
TTFELSGLQVQLINGSSYVPTTGQTSAAQTFGKVEDASSYLYPEG
LKRISKFIYPWLNSTDLKASTGDPDYGEPNFEYIPEGATDGSPQP
RLPASGGPGGNPGLYEDLFQVSVTVTNTGKVAGDEVPQLYVSLGG
PNEPKRVLRKFERLHIAPGQQKVWTTTLNRRDLANWDVVAQDWKI
TPYAKTIFVGTSSRKLPLAGRLPRVHLVSNQKMKHDVIFFQTPRL
LPRQRLNYSQSLRDTAHSHGTTLPSVRPSPETKKTNHQPHGTRRK
TEKKNMATTARASSPPGTRPFQPPTAALLVYPATLIIGSLFSVLS
PTAQGARASASDDAATAAAPVNYFARKNNIFNVYFVKIGWVWTTL
AFAAILLTQPAYTAPSAQRPRRLAQAAARYALATLVWWLTTQWFF
GPAIIDRGFVLTGGKCEARAEGTHAASSLPVSPLGMVSAAACKAA
GGAWTGGHDVSGHVFMLVLATAMLGFEMGGVFGVEGGKGVGVWSR
RFVGAVLGLSWWMLLMTAIWFHTWFEKLTGLLIALGTVYTVYILP
RRVVPWRNVVGIPGV
SEQ 24:
Polypeptide sequence of β-glucosidase 1 (BGL1)
homologue (Stachybotrys microspore)
MALASTFLSLYIFLFAYIFTGFACADPDSLYQRQDDGLLTSPPDY
PSPWADPEANGWEQAYAQARDFVSQLTLLEKVNLTTGVGWMSERC
VGNTGSIPRLGLRGLCLQDGPLGMRFTDYNSAFPAGVVAGATWSR
HLWHDRGRMMGEEQYGKGSDVLLGPASGPIGRAPTGGRNWEGFSV
DPYHSGVAMAQVVRGIQGAGVIATAKHFVANEQEHFRQAPEAVGY
GFNITESLSSNLDDKTLHELYAWPFQDAVRAGVGSIMCSYNQINN
SYGCQNSKLLNGILKDEYGFQGFVMSDWQAQHAGAASAAAGLDMS
MPGDTVFNTGYSFWGGNLTLGVINGTVPEWRIDDMALRIMAAFFK
VGRTVGGQPDINFSSWTRETLGYIHPMAQENLERVNFQVDVRGNH
ANHIRESAAKGSVILKNNGVLPLSSPKFVVVIGEDAGGNPAGPNG
CPDRNCDNGTLAMAWGSGTAQFPYLVTPDQALQRQALEDGTRYES
ILANNQWTAVQNILREPNTTTIVFADADSGEGFIDVDGNRGDRRN
LTLWKDGDALIKNVSSLTSNVIVVLHTVGPVLLTEWYDNPNITAI
VWAGVPGQESGNSLTDILYGRRSPGRSPFTWGRTRESYGADLLYE
PNNGEGAPQQDFSEGVFIDYRHFDRETAADPENTPIYEFGYGLSW
STFEYSNLQVAKRNVRPYQPTTGNTISAPVFGAPVDPDLSQYTFP
PGIRYIYRFIYPYLNTSSSGEDASGDPEYGQEADQFLPPGAIDGS
PQPRHPAGGQPGGNPQLWDVLYTVTATITNTGDRMTDEVPQLYIS
HGGDGEPVRVLRGFDRIERIAPGESVQFRADLTRHDLSNWDVVSQ
NWVITDDEKTVWVGSSSRNLPLAAPLR
SEQ 25:
Polypeptide sequence of β-glucosidase 1 (BGL1)
homologue (Rasamsonia emersonii)
MRNGLLKVAALAAASAVNGENLAYSPPFYPSPWANGQGDWAEAYQ
KAVQFVSQLTLAEKVNLTTGTGWEQDRCVGQVGSIPRLGFPGLCM
QDSPLGVRDTDYNSAFPAGVNVAATWDRNLAYRRGVAMGEEHRGK
GVDVQLGPVAGPLGRSPDAGRNWEGFAPDPVLTGNMMASTIQGIQ
DAGVIACAKHFILYEQEHFRQGAQDGYDISDSISANADDKTMHEL
YLWPFADAVRAGVGSVMCSYNQVNNSYACSNSYTMNKLLKSELGF
QGFVMTDWGGHHSGVGSALAGLDMSMPGDIAFDSGTSFWGTNLTV
AVLNGSIPEWRVDDMAVRIMSAYYKVGRDRYSVPINFDSWTLDTY
GPEHYAVGQGQTKINEHVDVRGNHAEIIHEIGAASAVLLKNKGGL
PLTGTERFVGVFGKDAGSNPWGVNGCSDRGCDNGTLAMGWGSGTA
NFPYLVTPEQAIQREVLSRNGTFTGITDNGALAEMAAAASQADTC
LVFANADSGEGYITVDGNEGDRKNLTLWQGADQVIHNVSANCNNT
VVVLHTVGPVLIDDWYDHPNVTAILWAGLPGQESGNSLVDVLYGR
VNPGKTPFTWGRARDDYGAPLIVKPNNGKGAPQQDFTEGIFIDYR
RFDKYNITPIYEFGFGLSYTTFEFSQLNVQPINAPPYTPASGFTK
AAQSFGQPSNASDNLYPSDIERVPLYIYPWLNSTDLKASANDPDY
GLPTEKYVPPNATNGDPQPIDPAGGAPGGNPSLYEPVARVTTIIT
NTGKVTGDEVPQLYVSLGGPDDAPKVLRGFDRITLAPGQQYLWTT
TLTRRDISNWDPVTQNWVVTNYTKTIYVGNSSRNLPLQAPLKPYP
GI
SEQ 26:
Polypeptide sequence of β-glucosidase 1 (BGL1)
homologue (Penicillium waksmanii)
MKLGWLEAATLAAASVASASDLAYSPPHYPSPWSTGDGEWAEAYR
RAVEFVSNLTLPEKVNLTTGAGWEQEKCVGETGGIPRLGMWGMCM
QDSPLGIRDSDYNSGFAAGVNVAATWDKRLAYQRGLAMGEEHRDK
GVDVQLGPVAGPLGRSPDGGRVWEGFSPDPVLTGVMMAQTIKGIQ
DAGVIACAKHFIGNEQEHFRQAGEAQGYGYNISESVSSNIDDKTM
HELYLWPFVDSVRAGVGSVMCSYNQINNSYGCSNSYTLNKLLKGE
LGFQGFVMSDWGAHHSGVGDALAGLDMSMPGDVILGSPYSFWGTN
LTISALNGTIPEWRLDDMAVRIMAAYYKVGRDRVRVPPNFSSWTR
DEYGYEHFIVSENQIKLNERVNVQRDHASGIRKLGSDSTVLLKNK
GALPLTHNERFVAILGEDAGSNPAGANGCSDRGCDDGTLAMGWGS
GTANFPYLITPEQAIQNEYLNYGNGQTNVFAVTNNSNTEQIAAMA
SQATVSLVFVNADSGEGYINVDGNEGDRKNLTLWKNGEQLIKTAA
ENCNNTIVIMHTPSAVLVGDWYDNENITAILWAGLPVSPIFSMVA
STPARRPPFTWGKTRDAYGAPLLTKPNNGQGAPQQDFSEGVFIDY
RQFDKADEEPIFEFGFGLSYTKFEFSDVHVTPLKADKYTKTTGRT
KPAPVLGKIGEASDYLFPSGIKRVTQYLYPWLNSTNLKESSGDPY
YGEKAEKYIPAHARDGSAQQLLPASGPSGGNAGLFEDLFQVTATV
TNTGSVVGDEVAQLYVSLGGEGDPVKVLRAFDRITIAPGQNAQWT
TTLTRRDLSNWDVASQNWVISDAPKKVYIGNSSRHLPVSIELPST
K
SEQ 27:
Polypeptide sequence of β-glucosidase 1 (BGL1)
homologue (Aspergillus udagawae)
MTLAEKVNLTTGTGIFMGPCAGQTGSALRFGIPNLCLHDSPLGVR
NSDHNTAFPAGITVGATFDKDLMYDRGAEMGEEFRGKGINVLLGP
SVGPIGRKPRGGRNWEGFGADPSLQAIGGAQTIKGIQSKGVIATI
KHYIGNEQEMYRMSNIGQRGYSSNIDDRTLHELYLWPFAEGVRAG
VGAVMAAYNDVNSSACSQNSKLLNEILKDELGFQGFVMTDWLGQY
GGVSSALAGLDMAMPGDGAVPLLGDAYWGSELSRSILNGSVPVSR
LNDMVTRIVATWYKMGQDGDYPLPNFSSNTQDATGPLYPGALFSP
SGVVNQYVNVQADHNITARAIARDAITLLKNDDNILPLRKNDSLK
IFGADAGPNPDGLNSCADQGCNKGVLTMGWGSGTSRLPYLVTPQQ
AIANISSNATFYITDSFPSNLAVGSGDIALVFINADSGENYITVE
GNPGDRTSAGLNAWHNGDKLVKDAAAKFSKVVVVIHTVGPILMEE
WIDLPSVKAVLVAHLPGQEAGWSLTDILFGDSSPSGHLPYTIPRS
ESDYPSSVGLLSQPLVQIQDTYTEGLYIDYRHFLKANITPRYPFG
HGLSYTTFSFSQPTLSVRTAVGSAYPPTRAPKGPTPSYPTTIPNP
SEVAWPKNFDRIWRYLYPYLDDPAGAAKNSSKTYPYPTGYTTVPK
PAPRAGGAEGGNPALFDVAFAVSVTVTNTGKRPGRAVAQLYVELP
NTLGVETPSRQLRQFAKTKVLAPGARETLTMEITRKDISVWDVVV
QDWKAPVQGQGVKFWLGESVLDMRAVCEVGGACTII
SEQ 28:
Polypeptide sequence of β-glucosidase 1 (BGL1)
homologue (Aspergillus transmontanensis)
MLLTVDAQRTIIRLSLRGQAMGEEFSDKGIDVQLGPAAGPLGAHP
DGGRNWEGFSPDPALTGVLFAETIKGIQDAGVIATAKHYIMNEQE
HFRQQPEAAGYGFNVSDSLSSNVDDKTIHELYLWPFADAVRAGVG
AVMCSYNQINNSYGCENSETLNKLLKAELGFQGFVMSDWTAHHSG
VGAALAGMDMSMPGDVTFDSGTSFWGANLTVGVLNGTIPQWRVDD
MAVRIMAAYYKVGRDTKYTPPNFSSWTRDEYGFAHNHVSEGAYER
VNEFVDVQRDHADLIRRIGAQSTVLLKNKGALPLSRKEKLVALLG
EDAGSNSWGANGCDDRGCDNGTLAMAWGSGTANFPYLVTPEQAIQ
NEVLQGRGNVFAVTDSWALDKIAAAARQASVSLVFVNSDSGEGYL
SVDGNEGDRNNITLWKNGDNVVKTAANNCNNTVVIIHSVGPVLIN
EWYDHPNVTGILWAGLPGQESGNSIADVLYGRVNPGAKSPFTWGK
TRESYGSPLVKDANNGNGAPQSDFTQGVFIDYRHFDKFNETPIYE
FGYGLSYTTFELSDLHVQPLNASQYTPTSGMTEAAKNFGEIGDAS
EYVYPEGLERIHEFIYPWINSTDLKASSDDSNYGWEDSEYIPEGA
TDGSAQPRLPASGGAGGNPGLYEDLFRVSVKVKNTGNVAGDEVPQ
LYVSLGGPNEPKVVLRKFERIHLAPSQEVVWTTTLTRRDLANWDV
SAQDWAVTPYPKTIYVGNSSRKLPLQVSLPKAQ
SEQ 29:
Polypeptide sequence of β-glucosidase 1 (BGL1)
homologue (Penicillium oxalicum)
MKLEWLEATVLAAATVASAKDLAYSPPFYPSPWATGEGEWAEAYK
KAVDFVSGLTLAEKVNITTGAGWEQERCVGSVMCSYNQINNSYGC
SNSYTLNKLLKGELGFQGFVMSDWGAHHSGVGDALAGLDMSMPGD
VILGSPYSFWGTNLTVSVLNSTIPEWRLDDMAVRIMAAYYKVGRD
RHRTPPNFSSWTRDEYGYEHFIVQENYVKLNERVNVQRDHANVIR
KIGSDSIVMLKNNGGLPLTHQERLVAILGEDAGSNAYGANGCSDR
GCDNGTLAMGWGSGTANFPYLITPEQAIQNEVLNYGNGDTNVFAV
TDNGALSQMAALASTASVALVFVNADSGEGYISVDGNEGDRKNMT
LWKNGEELIKTATANCNNTIVIMHTPNAVLVDSWYDNENITAILW
AGMPGQESGRSLVDVLYGRTNPGGKTPFTWGKERKDWGSPLLTKP
NNGHGAPQDDFTDVLIDYRRFDKDNVEPIFEFGFGLSYTKFEFSD
IQVKALNHGEYNATVGKTKPAPSLGKPGNASDHLFPSNINRVRQY
LYPYLNSTDLKASANDPDYGMNASAYIPPHATDSDPQDLLPASGP
SGGNPGLFEDLIEVTATVTNTGSVTGDEVPQLYVSLGGADDPVKV
LRAFDRVTIAPGQKLRWTATLNRRDLSNWDVPSQNWIISDAPKKV
WVGNSSRKLPLSADLPKVQ
SEQ 30:
Polypeptide sequence of β-glucosidase 1 (BGL1)
homologue (Histoplasma mississippiense)
MLLPIAFASLAVATEHLTESPPYYPSPWASGQGGWEDAVERARDF
VSQLTLVEKVNLTTGVGWMQENCVGQVGSIPRMGLHSLCMQDGPL
GIRFADYVSAFPAGVNVGATFSKELAYLRGKAMGEEHRDKGVDVV
LGPVVGPLGRSPDGGRNWEGFSPDPVNSGLLVAETIKGIQSASVI
ACVKHFIGNEQERFRQGPEAQGYGFDISESSSSNIDDVTMHELYL
WPFADAVRAGVGSVMCSYNQINNSYGCGNSYTQNKLLKAELGFQG
FIMSDWQAHHSGVGSALAGLDMSMPGDTVFGTGRSYWGPNLTIAV
ANGTIPEWRVDDMAVRIMAAYFKVGREAAKVPVNFNSWTRDEYDY
THALVKEGYGKVNERINVRAKHASIIRQVGAASVVLLKHTGSLPL
TGLEKNAAVIGEDAGPNLWGPNGCPDRRCDNGTLAMGWGSGTADF
PYLVTPAEAIQNEILSKGEGSVFPIFDNWASDQIKSAASQATVSL
VFVNADSGEGFISVDGNEGDRKNLTLWKGGDELIQTVASYCNNTV
VVIHSTGPVLVGEWNEHPNITAILWAGLPGQESGNSIADVLYGKV
NPGGRSPFTWGRTAEDYGASILKEPNEGNGAPQVDFTEGIFTDYR
AFDKADIKPIYEFGFGLSYTSFSYSDLNVEVPPWTLITDYRTKIT
SLLAQPMALHKNFFLLVGDPVEILVFMKYYIV
SEQ 31:
Polypeptide sequence of β-glucosidase 1 (BGL1)
homologue (Exophiala dermatitidis)
MHELYLWPFSDAVRAGTGSVMCSYNQINNSYGCANSYTMNHLLKN
ELDFQGFIMSDWQAQHSGVGTALAGLDMTMPGDTLFNTGRSYWGT
NLTIAVINGTVPEWRVDDMATRIMAAYYYVGRDTHYTPTNFYAWS
RNTYDKIHQVDPQSPIGLVNEHINVQDRHRDIVRQVGQASNVLLK
NTGGLPLTGKERQVGIFGYDAGSNPWGANGCSNRGCDNGTLAMGY
GSGTAEFPYLVTPEQAITQHVLTQTDGEVFAILDNYADAQIKSLA
STADVALVFANAQSGEGFITIDGNTGDRNNLSLWLGADRLIHNVT
KYNKNVIVVMHTVGPVNVSAWYDNENVTGIIWAGLPGQESGNAIV
DALYGLINPGGKLPFTIGRNREDYGTDILYEPNNGQFNAPQSLFS
EGVFIDYRHFDQYNIEPIYEFGFGLSYTTFEYSNLVITPGNPAPY
TPTSGQTEPAPVLGNASTDPSQYVFPNGTITYRPYLYIYPYLNST
DLRASSDDTDYGLPTDQYVPPGATDGSPQPLLPAGGAPGGNAGLY
EVVATVSATITNTGSVEGDEVAQLYVSLGEGEPPKVLRGFDRLTI
APGASTTFTANLTRRDVSVWDTVSQNWVQVSNPTIYVGTSSRKLP
LSGVLSSSGGGSGAQSSSSASGGSGGGSSSGWGYGQSSTASGSAP
APVSQLSDGQPQVPTGRPVTQVSDGQPQAPTGNPVSQISDGQPQA
PAHTGGAPVSQISDGQPQVPTGTGPAVTQISDGQPQNPTGGPAVS
QLSDGQPRATTA
SEQ 32:
Polypeptide sequence of β-glucosidase 1 (BGL1)
homologue (Aspergillus welwitschiae)
MRFTLIEAVALTAVSLASADELAYSPPYYPSPWANGQGDWAEAYQ
RAVDIVSQMTLAEKVNLTTGTGWELELCVGQTGGVPRLGIPGMCA
QDSPLGVRDSDYNSAFPAGVNVAATWDKNLAYLRGQAMGQEFSDK
GADIQLGPAAGPLGRSPDGGRNWEGFSPDPALSGVLFAETIKGIQ
DAGVVATAKHYIAYEQEHFRQAPEAQGYGFNITESGSANLDDKTM
HELYLWPFADAIRAGAGAVMCSYNQINNSYGCQNSYTLNKLLKAE
LGFQGFVMSDWAAHHAGVSGALAGLDMSMPGDVDYDSGTSYWGTN
LTISVLNGTVPQWRVDDMAVRIMAAYYKVGRDRLWTPPNFSSWTR
DEYGFKYYYVSEGPYEKVNQFVNVQRNHSELIRRIGADSTVLLKN
DGALPLTGKERLVALIGEDAGSNPYGANGCSDRGCDNGTLAMGWG
SGTANFPYLVTPEQAISNEVLKNKNGVFTATDNWAIDQIEALAKT
ASVSLVFVNADSGEGYINVDGNLGDRRNLTLWRNGDNVIKAAASN
CNNTIVIIHSVGPVLVNEWYDNPNVTAILWGGLPGQESGNSLADV
LYGRVNPGAKSPFTWGKTREAYQDYLYTEPNNGNGAPQEDFVEGV
FIDYRGFDKRNETPIYEFGYGLSYTTFNYSNLQVEVLSAPAYEPA
SGETEAAPTFGEVGNASDYLYPDGLQRITKFIYPWLNSTDLEASS
GDASYGQDASDYLPEGATDGSAQPILPAGGGAGGNPRLYDELIRV
SVTIKNTGKVAGDEVPQLYVSLGGPNEPKIVLRQFERITLQPSEE
TQWSTTLTRRDLANWNVETQDWEITSYPKMVFVGSSSRKLPLRAS
LPTVH
SEQ 33:
Polypeptide sequence of β-glucosidase 1 (BGL1)
homologue (Aspergillus eucalypticola)
MRFTLIEAVALTAVSLASADELAYSPPYYPSPWANGQGDWAQAYQ
RAVDIVSQMTLAEKVNLTTGTGWELELCVGQTGGVPRLGVPGMCL
QDSPLGVRDSDYNSAFPAGMNVAATWDKNLAYLRGKAMGQEFSDK
GADIQLGPAAGPLGRSPDGGRNWEGFSPDPALSGVLFAETIKGIQ
DAGVVATAKHYIAYEQEHFRQAPEAQGYGFNISESGSANLDDKTM
HELYLWPFADAIRAGAGAVMCSYNQINNSYGCQNSYTLNKLLKAE
LGFQGFVMSDWAAHHAGVSGALAGLDMSMPGDVDYDSGTSYWGTN
LTVSVLNGTVPQWRVDDMAVRIMAAYYKVGRDRLWTPPNFSSWTR
DEYGYKYYYVSEGPYEKVNQYVNVQRNHSELIRRIGADSTVLLKN
DGALPLTGKERLVALIGEDAGSNPYGANGCSDRGCDNGTLAMGWG
SGTANFPYLVTPEQAISNEVLKNKNGVFTATDNWAIDQIEALAKT
ASVSLVFVNADSGEGYINVDGNLGDRRNLTLWRNGDNVIKAAASN
CNNTIVVIHSVGPVLVNEWYDNPNVTAILWGGLPGQESGNSLADV
LYGRVNPGAKSPFTWGKTREAYQDYLVTEPNNGNGAPQEDFVEGV
FIDYRGFDKRNETPIYEFGYGLSYTTFNYSNLEVQVLSAPAYEPA
SGETEAAQTFGEVGNASDYLYPDGLQRITKFIYPWLNSTDLEASA
GDSSYGQDSSDYLPEGATDGSAQPILPAGGGPGGNPRLYDELIRV
SVTIKNTGKVAGDEVPQLYVSLGGPNEPKIVLRQFERITLQPSEE
TTWNTTLTRRDLANWNVEKQDWEITSYPKMVFVGSSSRKLPLRAS
LPTVH
SEQ 34:
Polypeptide sequence of β-glucosidase 1 (BGL1)
homologue (Aspergillus luchuensis)
MRFTLIEAVALTAVSLASADELAYSPPYYPSPWANGQGDWAQAYQ
RAVDIVSQMTLAEKVNLTTGTGYVLVRLAGFPADYNSAFPSGMNV
AATWDKNLAYLRGKAMGQEFSDKGADIQLGPAAGPLGRSPDGGRN
WEGFSPDPALSGVLFAETIKGIQDAGVVATAKHYIAYEQEHFRQA
PEAQGYGFNISESGSANLDDKTMHELYLWPFADAIRAGAGAVMCS
YNQINNSYGCQNSYTLNKLLKAELGFQGFVMSDWAAHHAGVSGAL
AGLDMSMPGDVDYDSGTSYWGTNLTVSVLNGTVPQWRVDDMAVRI
MAAYYKVGRDRLWTPPNFSSWTRDEYGYKYYYVSEGPYEKVNHYV
NVQRNHSELIRRIGADSTVLLKNDGALPLTGKERLVALIGEDAGS
NPYGANGCSDRGCDNGTLAMGWGSGTANFPYLVTPEQAISNEVLK
NKNGVFTATDNWAIDQIEALAKTASVSLVFVNADSGEGYINVDGN
LGDRRNLTLWRNGDNVIKAAASNCNNTIVIIHSVGPVLVNEWYDN
PNVTAILWGGLPGQESGNSLADVLYGRVNPGAKSPFTWGKTREAY
QDYLVTEPNNGNGAPQEDFVEGVFIDYRGFDKRNETPIYEFGYGL
SYTTFNYSNLEVQVLSAPAYEPASGETEAAPTFGEVGNASNYLYP
DGLQKITKFIYPWLNSTDLEASSGDASYGQDSSDYLPEGATDGSA
QPILPAGGGPGGNPRLYDELIRVSVTIKNTGKVAGDEVPQLYVSL
GGPNEPKIVLRQFERITLQPSEETKWSTTLTRRDLANWNVEKQDW
EITSYPKMVFVGSSSRKLPLRASLPTVH
SEQ 35:
Polypeptide sequence of β-glucosidase 1 (BGL1)
homologue (Aspergillus heteromorphus)
MRFGWIEAAALTAASVVSADELAYSPPFYPSPWANGQGDWASAYE
RAVAIVSQMTLAEKVNLTTGTGWELEKCVGQTGGVPRLGIPGMCG
QDSPLGVRDSDYNSAFPAGINIGATWDKNLAYLRGQAMGQEFSDK
GADFQLGPVAGPLGRAPDGGRNWEGFSPDPALTGVLFAETIKGIQ
DAGVVATAKHFIGYEQEHFREAPEAQGYGYNITESGSANIDDKTM
HELYLWPFADAVRAGVGAIMCSYNQINNSYACQNSYALNKLLKGE
LGFQGFVMSDWEAHHSGASAAMAGLDMSMPGDVVFDSGTSYWGTN
LTIGVLNGTVPQWRVDDMAVRIMAAYYKVGRDRLWTPPNFSSWTR
DEYGFKYYYSSEGPYEVVNHFVDVRRNHSDLIRRIGADSTVLLKN
DGCALPLTGTERKVALIGEDAGSNPYGADGCSDRGCDNGTLAMGW
GSGTTEYPYLVTPEQAIQAEVIQNGGTVFAVTDNWAISQMETLAA
EATVSLVFVNADSGEGYIDVDGNMGDRNNLTLWGNGDNVIKAAAS
NCNNTIVIIHSVGPVLVNEWYDHPNVTAILWAGLPGQESGNSLAD
VLYGRVNPGAKSPFTWGKTRESYQDYLITEPNNGDGAPQEDFTEG
VFIDYRGFDKRNETPIYEFGYGLSYTTFNYSQLQVQALNASAYTP
ASGETEAAPTLGEAGNASDYLYPSGLQRITAFIYPWLNSTDLEAS
SGDSSYGQSSDFLPEGATDGSAQPILPAGGGPGGNPALYDDLIQV
SVTIKNTGDIAGDEIPQLYVSLGGPDEPRIVLRKFDRITLQPSEE
YEWTTTLTRRDLSNWDVAAQDWIVTSYPKKVYVGSSSRKLPLRAS
LPTVQ
SEQ 36:
Polypeptide sequence of β-glucosidase 1 (BGL1)
homologue (Aspergillus aculeatus)
MKLSWLEAAALTAASVVSADELAFSPPFYPSPWANGQGEWAEAYQ
RAVAIVSQMTLDEKVNLTTGTGWELEKCVGQTGGVPRLNIGGMCL
QDSPLGIRDSDYNSAFPAGVNVAATWDKNLAYLRGQAMGQEFSDK
GIDVQLGPAAGPLGRSPDGGRNWEGFSPDPALTGVLFAETIKGIQ
DAGVVATAKHYILNEQEHFRQVAEAAGYGFNISDTISSNVDDKTI
HEMYLWPFADAVRAGVGAIMCSYNQINNSYGCQNSYTLNKLLKAE
LGFQGFVMSDWGAHHSGVGSALAGLDMSMPGDITFDSATSFWGTN
LTIAVLNGTVPQWRVDDMAVRIMAAYYKVGRDRLYQPPNFSSWTR
DEYGFKYFYPQEGPYEKVNHFVNVQRNHSEVIRKLGADSTVLLKN
NNALPLTGKERKVAILGEDAGSNSYGANGCSDRGCDNGTLAMAWG
SGTAEFPYLVTPEQAIQAEVLKHKGSVYAITDNWALSQVETLAKQ
ASVSLVFVNSDAGEGYISVDGNEGDRNNLTLWKNGDNLIKAAANN
CNNTIVVIHSVGPVLVDEWYDHPNVTAILWAGLPGQESGNSLADV
LYGRVNPGAKSPFTWGKTREAYGDYLVRELNNGNGAPQDDFSEGV
FIDYRGFDKRNETPIYEFGHGLSYTTFNYSGLHIQVLNASSNAQV
ATETGAAPTFGQVGNASDYVYPEGLTRISKFIYPWLNSTDLKASS
GDPYYGVDTAEHVPEGATDGSPQPVLPAGGGSGGNPRLYDELIRV
SVTVKNTGRVAGDAVPQLYVSLGGPNEPKVVLRKFDRLTLKPSEE
TVWTTTLTRRDLSNWDVAAQDWVITSYPKKVHVGSSSRQLPLHAA
LPKVQ
SEQ 37:
Polypeptide sequence of β-glucosidase 1 (BGL1)
homologue (Aspergillus caelatus)
MKLGWFEVAALAAASVVSAQDDLAYSPPFYPSPWADGQGEWAEVY
KRAVEIVSQMTLTEKVNLTTGTGWQLERCVGQTGSVPRLNIPSLC
LQDSPLGIRFSDYNSAFPAGVNVAATWDKTLAYLRGQAMGEEFSD
KGIDVQLGPAAGPLGAHPDGGRNWEGFSPDPALTGVLFAETIKGI
QDAGVIATAKHYILNEQEHFRQQPEAAGYGFNVSDSLSSNVDDKT
IHELYLWPFADAVRAGVGAVMCSYNQINNSYGCENSETLNKLLKA
ELGFQGFVMSDWTAHHSGVGAALAGMDMSMPGDVTFDSGTSFWGA
NLTIGVLNGTIPQWRVDDMAVRIMAAYYKVGRDTKYTPPNFSSWT
RDEYGFAHNHVSEGAYERVNEFVDVQRDHADLIRRIGAESTVLLK
NKGALPLSRKEKLVALLGEDAGSNPWGANGCDDRGCDNGTLAMAW
GSGTANFPYLVTPEQAIQNEVLQGRGNVFAVTDSWALDKIAAAAR
QASVSLVFVNSDSGEGFLSVDGNEGDRNNITLWKNGDNVVKTAAN
NCNNTVVIIHSVGPVLINEWYDHPNVTGILWAGLPGQESGNSIAD
VLYGRVNPGAKSPFTWGKTRESYGSPLVNEANNGNGAPQSDFTQG
VFIDYRHFDKFNETPIYEFGYGLSYTTFELSDLHVQPLNASQYTP
TSGMTEAAKNFGETGDASEYVYPEGLERIHEFIYPWINSTDLKAS
SDDSNYGWEDSKYIPEGATDGSAQPRLPASGGAGGNPGLYEDLFR
VSVKVKNTGKVAGDEVPQLYVSLGGPNEPKVVLRKFERIHLAPSQ
EVVWTTTLTRRDLANWDVSAQDWTVTPYPKTIYVGNSSRKLPLQA
SLPKAR
SEQ 38:
Polypeptide sequence of β-glucosidase 1 (BGL1)
homologue (Madurella mycetomatis)
MANLVLALLLFLSALTSAATDTPFLGYPSPWATNITDPTWAAAYA
QAIAFVANLTLTEKVNLTTGTGWEADRCIGATGGIPRLGFRPFCL
MDGPLGVRYTDHNSAFPAGVNTAATFSRRLMRLRGEAMGAEFRGK
GIDVMLGPVAGALGRVPQGGRNWEGFSPDPYLTGVAMAETIQGIQ
SRGVVACAKHYILNEQEHFRGSIDVRIDDRTMHELYLWPFADAVR
AGVGSVMCSYNKINGTYACENEWTTNYLLKNELGFQGFVLSDWGA
QHNTLGSALGGLDMAMPGDGGPPPYRAWWGGALTEAVLRGDVPQW
RLDDMAVRIMAAYFRVHTGNYTSRPDINFSAWTNSTVGPLYPAAN
QSYTVVNEFVDVQSDHASLIREIGAKSVVLLKNAYDLLPLRQPGP
PIIAVIGDDAQDHPLGPNACPERGCLNGTLAMGYGSGTANFPYLV
SPLTALTEQARADNTTLLYAPSNWDLDAAITTARNASIAIVFAAA
TSGENFITVDGNAGDRNNLTLWANGDALIKAVASINPNTIVVLHT
PGPVILDYAEEHPNISAILWAGLPGQESGNALVDVLYGKVNPQGR
SPFTWGDSVEEYGAQLMFEAENPRAPVQSFDEGVFIDYRKFSTFG
GKLTYPFGFGLSYTKFRYSGLSVVRKEEVEGRFEPAEGLTGPAPT
FGVVEAELGVHTAPEGFTRISPYVYPWLNNSESLVTGNSTGASEF
PAAARNGSAQPVLPASGAPGGNPGLYEVLYTITASIENVGEVAGT
EIPQLYVQLGGEENPFGVLRGFDEVELEPGETKNVTFELTRRDVS
NWNTSTQNWEITDREKVVFVGSSVRDIRLNASLPAPVGMAWRHG
SEQ 39:
Polypeptide sequence of β-glucosidase 1 (BGL1)
homologue (Aureobasidium pullulans)
MSQSQDYELTPRESPRQSLDHPEDYRLSTSSRDSISSSINELDPL
KTPNRPYKDDVSQTHDSVFVTRRRSTWRRYLLPSRMCCMMVLLFT
AALVMLLSAGGIWVYKTEPEGGQSDPWYPAPRGGSVTAWEEAYKK
AAALVGQMSVVEKVNITTGTGWEMGMCVGNTGPVERLGFPSLCLQ
DGPLGLRFADNITAFPAGITLGTTWNKELIYLKGRAHGREARGKG
VHIALGPSMGPFGRLPAGGRNWEGFGSDPVLQGLAAAAMIRGIQE
EGIIATAKHYIANEQEHFRQSWEWGTPNAISSNLDDRTLHEIYAW
PFAESVKAGVGSVMCSYNLVNQSYACQNSKLLNGILKDELGFQGF
IQSDWLAQRSGVASALAGLDMSMPGDGLRWMDGQSVWGGELTKAV
FNGSVPMERLNDMVLRVVAAWYQFGQDDKKKWPAEKDGGGPNFSS
WTNDEIGLLHPGSNDTTKGVVNKFVNVQGEGDDSHGKLARQIAAE
GTVLLKNDDHVLPLNRDGKKLTKNGDKLRVAIFGEDAYNNGKGPN
ACADRACNEGTLAQGWGSGTVEFPYLVAPAEGIHLGFDVESVTLS
DYPSNYEDLDKHPSRAADQDLCFVFINSDGGEGYVAWKDVKGDRN
DLYAQKGGDELVQKVAAKCGGPTIVVVHAVGPVILEKWIDLPGVK
AVLYANLPGQESGNALASIVFGDINPSGRLPYTIAKNEDDYGPTS
KILYRPNAVIPQQNFSEGLYVDYRYFDKHDIEPRYEFGFGLSYTT
FHLSSLFIHPLATEYSTLPAKRPESITPPHLDVSIPDAREALWPS
NFIALKKYIYPYINSVDDIKTGKYPYPVGYEIAQPLSQAGGAEGG
NPDLYTPLAVVQASLTNTGSLPGDCVVQLYISYPSTPIIDSLGNT
VDFPVRVLRAFDKLFVTPEKRVEVSLQLTRKDLSFWDVGLQNWVL
PMGDFEVQLGFSSRDIQQKGDILSFSRLLL
SEQ 40:
Polypeptide sequence of β-glucosidase 1 (BGL1)
homologue (Fusarium oxysporum)
MALIICFYYPISLGEAPDSDHTLKEKILGLGLKSAVILAGALVCL
FLALQWGGTKHPWSDSRVWGCLVGFGLLLTLFVYIQIRQGEAALI
RPRIISQRSVFLGCLFSALYQGAMTTQSYHLPFYFQAVKGVDPQS
SGVDILPHGVTVTIATLITGSIITWLGYYVPFMWAGSAIFTIGAG
LLYTISQNTPLARWFGYEVLAGAGFGIAIQIPIFAVQVVLVAGDI
PLGTVLIILSQALGGSVGLSISQNVFQNSLRQRLNTIADIDIQVV
IAAGGTDLEHVVSADSLAHVRDAFRYGISNAFLVSTALGGVAVLA
SIGMERKKIKSKKEGVDLYKTARPFDVSVFSSRGSIDTKMTLAKV
AFTLANLVAVGSSAKNVADGFVAAPYYPAPYGGWDEAWADSYARA
KKMVDSMTLAEKTNITAGTGLFMADRVGFPQLCLNDAANGVRLAD
NVTVFPDGITAGATFDKKLMYERGVAIGKEARGKGVNVWLGPSVG
PIGRKPKGGRNWEGFGADPSLQGIGARETIKGVQEQGVIATVKHL
IGNEQEMYRRQTTDVVSPAYSANIDDRTMHELYLWPFAEAVKVGV
GATMTAYNRVNGTISSEHSYLINALLKEELGFQGFVMTDWLSQIT
GVQSAIAGMDMSMPGDTIIPLFGNSLWMYELTRSALNGSVPMSRL
NDMATRIVATWYQFEQDKDFPSVNFDTNTYNRVGPLYPAAWPNSP
SGVVNQYVQVQDDHDEIARQIAQDAITLLKNDEKLLPLSTKSSLK
VFGTGAQTNPDGANACVDRSCNKGTLGQGWGSGTVDYMYLDDPIG
AIKKAAGDVTFYNTDKFPSVPSPSDDDVAIVFVTSDSGENQYTVE
GNNGDRNADKLNVWHNGDALIKAAAAKYKNVVVVIHTVGPVLVDQ
WIDLPSVKSVLVAHLPGQEAGKSLTNILFGDASPCGHLPYSITKK
EDDMPESVTKLIDSGFIDAPQDTYSEGLFIDYRWLNKEKIQPRYA
FGHGLSYTNFTYTNATIKRGTQLSQYPPKRPAKGKVLDYSQDIPD
YKEAIKPSSFKTVWRYLYSWLSESDAKSAAAKAETSKYPYPDGYS
TAQKTALPKAGGVSGGNPALWDEAYTLSVVVTNAGSKFSGKASVQ
AYVQFPSVAGYETPVIQLRDFEKTKVLEPGSSETVQLTLTRKDLS
VWDVKAQDWLVLDGEFKIWLGSASDKLDAVCFTDDLGCEHDVKGP
VSYDS
SEQ 41:
Polynucleotide sequence of exoglucanase
(cex-like) homologue (Cellulomonas wangleii)
ATGACCCCCCCACCCCAGCTCCGACGCGGGCGCCCCCTGGTCGCA
CGCGGCGCCTCCGCCATCGCGATCGTCGCGCTCGCGGTGACGGCC
GCCGCGGCCATCCCGGCGCAGGCCGCCGGCTCGACGCTCCAGGAG
GCCGCCGCCCAGAGCGGCCGCTACTTCGGCACCGCGATCGCCGCG
AACAAGCTCAGCGACTCGACCTACGTGACCATCGCGAACCGTGAG
TTCAACATGATCACGGCCGAGAACGAGATGAAGATGGACGCGACG
GAGCCGAACCAGAACCAGTTCAACTACAGCCAGGGCGACCGGATC
CTCAACTGGGCCAAGCAGAACGGCAAGCAGGTCCGCGGGCACGCG
CTCGCGTGGTACTCCCAGCAGCCGGAGTGGATGAAGCGCATGGAG
GGCTCCGCGCTGCGCAGCGCGATGGTCAACCACGTCACCCAGGTG
GCGACGCACTACAAGGGTCAGATCTACGCCTGGGACGTCGTGAAC
GAGGCGTTCGCCGACGGCAGCTCCGGCGGACGCCGCGACTCCAAC
CTGGAGCGCACCGGCAGCGACTGGATCGAGGTGGCCTTCCGCGCC
GCGCGCGCCGCCGACCCGGCCGCGATCCTCTGCTACAACGACTAC
AACATCGACGACTGGTCGCACGCCAAGACCCAGGGCGTCTACCGG
ATGGTCCGTGACTTCAAGCAGCGCGGCGTCCCGATCGACTGCGTC
GGCCTGCAGTCGCACTTCAACCCGCAGAGCCCGGTCCCGGCCAAC
TACCAGACGACGATCGAGAGCTTCGCCGCCCTCGGCGTCGACGTG
CAGATCACCGAGCTCGACATCGAGGGCTCCGGCTCGGCGCAGGCC
GAGAACTTCCGCAAGGTGACCCAGGCCTGCCTCAACGTCGCCCGC
TGCACCGGCATCACGGTGTGGGGCGTGCGTGACACGGACTCGTGG
CGCGCCTCGGGCACCCCGCTGCTGTTCGACGGCAACGGCAACAAG
AAGCAGGCGTACACCTCGGTGCTCAACACGCTGAACGCGGCGAGC
CCGACCACGCCGCCGCCCACCACGCCGCCGCCGACGACCCCGCCG
CCGGTGACGCCCCCGCCCACGACCCCGCCGCCCACCACGCCGCCG
CCGACGACCCCGCCGCCGACGACCCCGCCGCCGGTGACGGGCAGC
TGCTCCGCCGCACTGACCATCGCCAACGCGTGGCCCGGTGGCTAC
CAGGGCACGGTCACGGTGACGGCCGGCTCGTCGTCCCTCAACGGG
TGGCGGGTGACCCTGCCGGGTGGCGTCTCCACGAACAACGTGTGG
AACGGCGTGGTCTCCGGCAACGTCGTGAGCAACGCGCCCTACAAC
GGCTCCGTCGGGGCCGGGCAGTCGACGACCTTCGGGTTCATCGGG
AACGGCAACGCCCCGTCGCCGACCTCCCTCACCTGCTCCTGA
SEQ 42:
Polynucleotide sequence of exoglucanase
(cex-like) homologue (Cellulomonas shaoxiangyii)
ATGCTCCGACGAGGGAGCAGGCGCGGCCCCGGCGCTCGAGGCGCA
GCCGTCCTCGCCGCCCTGACCCTCGCGGCGACCGCCGCGGCCGCC
ATCCCGGCTCAGGCCGCCGGCTCGACGCTGCAGGACGCGGCCAAC
GACCGCGGCCGCTACTTCGGCACGGCGATCGCCGCGAACCGCCTC
AGCGACTCGACCTACTCGACCATCGCGAACCGTGAGTTCGACATG
ATCACGGCCGAGAACGAGATGAAGATGGATGCGACGGAGCCGTCG
CAGAACCAGTTCAACTACACCAACGGCGACCGCATCGTGAACTGG
GCGCTCCAGAACGGCAAGCAGGTCCGCGGGCACGCGCTGGCGTGG
CACTCGCAGCAGCCGGGCTGGATGCAGAACATGTCCGGCACGGCG
CTGCGCAACGCCATGCTCAACCACGTCACGCAGGTCGCCACGCAC
TACCGCGGCAAGATCTACGCCTGGGACGTCGTGAACGAGGCCTTC
GCCGACGGCTCGTCCGGCGCCCGCCGCGACTCCAACCTGCAGCGC
ACCGGCAACGACTGGATCGAGGCGGCCTTCCGCGCCGCCCGGGCC
GCCGACCCCGGCGCGAAGCTCTGCTACAACGACTACAACACCGAC
GACTGGACGCACGCGAAGACGCAGGCCGTGTACAACATGGTCCGC
GACTTCAAGGCGCGCGGCGTCCCGATCGACTGCGTCGGCCTGCAG
TCGCACTTCAACGCGCAGAGCCCGGTGCCGAGCAACTACCAGACG
ACCCTGTCGAGCTTCGCCGCGCTCGGCGTGGACGTGCAGATCACC
GAGCTCGACATCGAGGGCTCGGGCTCCGCGCAGGCGGAGAGCTAC
CGGCGCGTCGTCCAGGCGTGCCTCAACGTCTCCCGCTGCACCGGC
ATCACGGTGTGGGGCGTGCGGGACACCGACTCGTGGCGCGCCTCG
GGCACGCCGCTGCTCTTCGACGGCCAGGGCAACAAGAAGGCGGCG
TACACCGCGGTCCTCGACACGCTGAACAGCGGACCCGGGACCAAC
CCGACGACGCCTCCGCCCACCAGCCCCCCGCCGACGACCCCGCCG
CCGGTCACCCCGCCGCCGACGACGCCCCCGCCCACGACCCCGCCG
CCGGTCACCCCGCCGCCCAGCGGCACCTGCTCCGCGGCACTGACG
ATCGCGAACAGCTGGGGCGGCGGCTACCAGGCCACCGTCACGGTG
CGGGCCGGTTCGTCCGCTCTCAACGGATGGCGGGTCACCCTGCCC
GGCGGCGTCACGACGTCGAACCTGTGGAACGGCGTCCTCGCCGGC
AGCGTCGTGACGAACGCGCCGTACAACGGCTCCGTCGGAGCCGGC
CAGTCGACGTCCTTCGGGTTCATCGGCAACGGCAGCGCACCCGCC
GCGGGAGCGCTGAGCTGCAGCTGA
SEQ 43:
Polynucleotide sequence of exoglucanase
(cex-like) homologue (Micromonospora sp.
WMMA1998)
ATGGACAAGGTGCTCGCCCGCGGCAGCGGGAGCCCGACCGCCCGA
TACCGGCCGCGTGCCGCCCTGCTGTCGGCCGCCGTCGGCGCGGCG
CTGGTCGCGGCCACGGTCGCGATGGCGACCAGCGCCAGCGCCGGG
ACCACCCTGGGCGCGGCGGCGGCGGAACAGGGCCGCTACTTCGGC
ACCGCGGTGGCCGCGAACAAGCTGTCGGACGCCACGTACGTCGGC
ATCCTGAACCGCGAGTTCACTATGGTCACCCCCGAGAACGAGATG
AAGTGGGACGCCACCGAGCCGTCCCAGGGCCAGTTCAGCTACACC
AACGCCGACCGGATCGTCGCCCACGCCCAGGCCAACGGCATGCGG
GTACGCGGCCACGCGCTGGCCTGGCACTCGCAACAGCCCGGCTGG
GCGCAGAACCTGTCCGGCAGCGCGCTGCGCCAGGCGATGGTCAAC
CACATCACCCAGGTCGCCACCCACTACAAGGGCAAGATCTACGCC
TGGGACGTGGTCAACGAGGCGTTCGACGACGGCAGCGGCGGCCGG
CGCGACTCCAACCTCCAGCGCACCGGCAACGACTGGATCGAGGTG
GCGTTCCGCACCGCGCGCGCCGCCGACCCGGGCGCCAAGCTCTGC
TACAACGACTACAACACCGACAACTGGACCTGGGCCAAGACCCAG
GGCGTCTACAACCTGGTCAAGGACTTCAAGGCCCGGGGCGTGCCG
ATCGACTGCGTCGGCCTGCAGTCGCACTTCAACAGCGGCTCGCCG
TACCCGAGCAACTACCGCACCACGTTGCAGAACTTCGCCGCCCTC
GGCGTCGACGTGCAGATCACCGAACTCGACATCGAGGGTTCGGGC
AGCGCCCAGGCCACCACGTACGCCAACGTGGTCAAGGACTGCCTG
GCCGTGTCGCGGTGCACCGGCATCACCGTCTGGGGAATCCGGGAC
AGCGACTCCTGGCGGGCCGGCGGCACCCCGCTGCTCTTCGACGGC
AACGGCAACAAGAAGGCGGCCTACACCGCCGCGCTCGACGCGCTC
AACGCCGGCGGCACGCCGCCGCCGACCACCACGCCGCCGACCACC
ACGCCCCCGACCACGACTCCGCCGACGACCACGCCGCCCACCACC
GCGCCGCCCACGACTCCCCCGCCCACCGGCGGCGGGTGCACCGCG
TCGCTCACCACCAACCAGTGGCCCGGCGGGTTCGTCACCACCGTG
CGCGTCACCGCCGGCGCCGGCGCGCTCAACGGCTGGACGGTGACG
CTCACCGTGCCGTCCGGCTCGGCGGTCACCAACACGTGGAGCGCC
CAGGCCAGCGGCGCCAGTGGCGCGGTGACGTTCCGCAACGTCGCC
TACAACGGCCAGGTCGGTGCCGGCGGCAGCACCGAGTTCGGCTTC
CAGGGGACCGGCACGGCCCCCTCCGGCACGCCCACCTGCGCGGCG
GGCTGA
SEQ 44:
Polynucleotide sequence of exoglucanase
(cex-like) homologue (Xylanimonas cellulosilytica)
ATGAACCATCAGCACCACCGTCGTAGGTTGCGCAGCGTCGCAGCC
GTCGCGCTCGCCTCCCTGGTCGTCGCCACGGGAGTGGTGACCGCC
CAGGCGGCCGGATCCACGCTCCAGGAAGCGGCGGGCAGCCGCTAC
TTCGGCACGGCCATCGCGGCCAACAAGCTCTCGGACTCGACCTAC
TCGACCATCGCGAACCGTGAGTTCGACATGATCACGGCCGAGAAC
GAGATGAAGATGGACGCCACCGAGCCGTCTCAGGGCAGCTTCAAC
TTCACCAACGCCGACAGGATCGTCGACTGGGCCACCGCCAACGGC
AAGCGGATGCGCGGTCACGCCCTCGCGTGGCACTCGCAGCAGCCG
GGGTGGATGCAGAACATGTCGGGCACCGCGCTGCGCACGGCGATG
CTCAACCACGTCACCGAGGTCGCTGCGCACTACAAGGGCAAGATC
TACGCCTGGGACGTCGTCAACGAGGCCTTCGCCGACGGGTCCTCG
GGCGCCCGCCGCGACTCGAACCTGCAGCGCACGGGCGACGACTGG
ATCGAGGCCGCGTTCAGGGCCGCCCGGGCCGCCGACCCGTCCGCC
AAGCTCTGTTACAACGACTACAACACGGACAACTGGAACTGGGAG
AAGACCCAGGCCGTGTACGCCATGGTCAAGGACTTCAAGGAACGC
GGCGTGCCGATCGACTGCGTCGGGCTCCAGTCCCACTTCAACTCG
GGCAGCCCCTACCCGAGCAACTACCGCACGACGCTGCAGAACTTC
GCGGCGCTCGGCGTCGACGTCCAGATCACCGAGCTGGACATCGAG
GGCTCGGGCAGCACGCAGGCCGACACGTACGCCAAGGTGGTCGCC
GACTGCCTCGCGGTGAGCCGCTGCACCGGCATCACGGTGTGGGGC
GTGCGCGACTCCGACTCGTGGCGCGCGAGCGGTACGCCGCTGCTG
TTCGACGCGTCGGGCAACAAGAAGGCCGCCTACACGTCGGTGCTG
AACACCCTCAACGGCGGTGCGACGCCGACGCCGACGCCGACGCCG
ACGCCGACGCCGACCCCGACTCCCACGCCGACTCCGACCCCGACG
CCGACGCCGACTCCCACGCCGACGCCGACGCCGACGCCGACTCCC
ACGCCGACGCCGACGTCCGGTCCGTGCACCGCCACGATGACGATC
ACGAACTCGTGGCAGGGCGGCTTCCAGGGTGAGGTCACGGTCAAG
GCGGGCAGTGCCCGCACGTCCTGGTCGACGTCGTTCACCTCGGCC
GCCACGGTCCAAGTCTGGAACGGGGTGCACTCGACCTCCGGCTCG
GTGCACACGGTGTCGAACCAGCCGTACAACGGCACGCTGGCCGCC
GGTGCGTCGACGACGTACGGCTTCACGGCCACCGGGACGGCGCCG
AGCAGCCCGCCCGCGGTGACCTGCTCGTGA
SEQ 45:
Polynucleotide sequence of exoglucanase (cex-like)
homologue (Verrucosispora sp.
WMMD573)
ATGAACAACGCCCACGCCCGCGCCAGCGGGCGGCCGGCTACCCGA
CTGCGACCGCGCGCCGCGCTGGTCTCGGCCGCGGTCGGTCTCGCC
CTCGTCGCCACCAGCATGGTGGTGACCTCCAGCGCCCAGGCCGCC
GAGTCCACCCTCGGTGCGGCCGCGGCCCAGTCCGGCCGGTACTTC
GGTGCCGCGGTGGCGGCGAACAAGCTGTCCGACTCCACCTACGTC
GGCATCCTGAACCGCGAGTTCAACTCGGTCACGGCCGAGAACGAG
ATGAAGATCGACGCGCTCGAGCCGCAGCAGAACAACTTCACGTTC
GGCAACGCGGACCGGATCGTCAACCACGCCCTGTCCCGGGGCTGG
AAGGTCCGGGGGCACACCCTGGCCTGGCACTCGCAGCAGCCTGGC
TGGATGCAGCAGATGGAAGGCACGGCGCTGCGTAACGCGATGCTG
AACCACGTCACCCGGGTCGCGACCTACTACCGCGGCAAGATCGAC
TCGTGGGACGTGGTGAACGAGGCGTTCGACGACGGCAACAACGGC
GCGCGCCGTAACTCGAACCTGCAGCGCACCGGTAACGACTGGATC
GAGGCCGCGTTCCGGGCCGCCCGGGCCGCCGACCCGGGCGCCAAG
CTCTGCTACAACGACTACAACACCGACAACTGGACCTGGGCCAAG
ACTCAGGCCGTCTACCGGATGGTGCAGGACTTCAAGCAGCGTGGC
GTGCCGATCGACTGCGTCGGCCTCCAGTCGCACTTCAACAGCGGC
TCGCCGTATCCGAGCAACTACCGCACCACGCTGTCCAGCTTCGCC
GCGCTCGGCGTGGACGTGCAGATCACCGAGCTGGACATCGAAGGC
TCCGGCAGCACCCAGGCCAACACCTACCGCAACGTGGTCAACGAC
TGCCTCGCGGTGCCCCGCTGCAACGGCATCACCGTGTGGGGAATC
CGGGACACCGACTCCTGGCGTTCCGGTGGCACCCCGCTGCTCTTC
GACGGCAACGGCAACAAGAAGCAGGCGTACGACGCGACGCTCCAG
GCGCTGAACTCGGGCGGCACCACCCCGCCGCCCACGACCCCGCCG
CCGCCCACGACCCCGCCGCCCACCACGCCGCCGCCCACGACCCCG
CCGCCGACCACCCCGCCGCCCGGCGGGTCCGGCTGCACGGCAACG
GTGTCCCTGAACCAGTGGAACGGTGGTTTCGTCGCCACGATCCGC
GTCACCGCCGGTTCCGCGAGACTGAACGGCTGGTCGGTGGGCATC
GGGATTCCGGGCGGTAGCTCGGTGACCAACACCTGGAACGCCCAG
GCCAGCGGCAACAGCGGCAACGTGACGTTCCGCAACGTCAGCTAC
AACGGCACCGTGAACGCCGGTGCCACCACGGAGTTCGGCTTCCAG
GGCACCGGTACCGGTCCATCCGGTACCCCCACCTGCACCGGTAGC
TGA
SEQ 46:
Polynucleotide sequence of exoglucanase
(cex-like) homologue (Saccharothrix syringae)
ATGTCCAGAACTGCTGTCACAGCTGCCGGCAGGTCGGAGGCACGA
GGCCGGTCGCGGCGCGCCGCGGTGGTGGTCGGTGCGATCGGGTTG
CTGAGCGCGGCGGCCGTGGTGCTGCCGAACGTGGCCACCGCCGGC
ACCACGCTGGGCGCGTCCGCCGCGGAGAGCGGGCGCTACTTCGGC
ACGGCGGTCGCGGCCAACAAGCTCTCGGACTCGACCTACGTGGGC
ATCCTCAACCGTGAGTTCGACATGGTCACGGCTGAGAACGAGATG
AAGATGGACGCCACGGAGCCGAACCAGAACCAGTTCTCGTTCGGC
AACGGTGACCGGATCGTCAACCACGCCCGCAACCAGGGCAAGCGG
GTCCGTGGCCACGCGTTGGCGTGGCACTCGCAGCAGCCGGGCTGG
ATGCAGAACATGTCCGGCACGGCGTTGCGCAACGCGATGCTCAAC
CACGTCACCCAGGTCGCCACGTACTACAAGGGCAAGATCTACGCC
TGGGACGTGGTCAACGAGGCGTACGCCGACGGCAGCTCCGGCGGT
CGGCGTGACTCGAACCTCCAGCGGACCGGCAACGACTGGATCGAG
GCCGCCTTCCGGGCCGCCCGCGCCGCCGACCCGAACGCGAAGCTG
TGCTACAACGACTACAACACCGACAACTGGTCGCACGCCAAGACC
CAGGGCGTGTACCGGATGGTGCAGGACTTCAAGTCGCGCGGTGTG
CCGATCGACTGCGTCGGCTTCCAGGCGCACTTCAACAGCGGCAAC
CCGGTGCCGTCGAACTACCACACCACCCTGCAGAACTTCGCCGAC
CTCGGCGTGGACGTCCAGATCACCGAGCTGGACATCGAGGGCTCC
GGCAGCACCCAGGCCCAGCAGTACCAGGGCGTGGTCCAGGCGTGC
CTCGCGGTGACCCGCTGCACCGGCATCACGGTGTGGGGCATCCGC
GACAGCGACTCGTGGCGTTCCTCGGGCACCCCGCTGCTGTTCGAC
GGCTCGGGCAACAAGAAGGCCGCCTACACCTCGGTCCTCAACGCC
CTCAACGCGGGCAGCACCGCGCCGCCGTCCACCACCACGACGACG
ACCCCGCCGAACACCTCGGACTGCCGCGCCGGCTACGTCGGCCTG
ACCTACGACGACGGCCCCAACGGCAGCACCACCACGCAGCTGCTC
AACGCGCTGCGGTCGGCCGGCCTGCGCGCCACGTTCTTCAACCAG
GGCAACCGGGTCCAGCAGAACCCCGGCCTGGCCAAGGCGCAGCGC
GACGCCGGCATGTGGGTCGGCAACCACAGCTGGAGCCACCCGCAC
ATGACCCAGCTGAGCCAGTCGCAGATGGCCTCGGAGATCTCCCAG
ACCCAGCAGGCCATCCAGTCGGCCACCGGTGAGGCGCCGAAGCTG
TTCCGCCCGCCCTACGGCGAGACCAACAGCACGCTCAAGTCGGTC
GAGGCCCAGTACGGCCTGACCGAGGTGCTGTGGAGCGTCGACTCG
CAGGACTGGAACAACGCCAGCACCGCGCAGATCGTGCAGGCCGCG
TCGACCCTGCAGAACGGCGGCGTGATCCTGATGCACGACGGCTAC
CAGACGACGATCAACGCCATCCCCCAGATCGCGGCCAACCTCGCC
AGCCGCGGCCTGTGCGCCGGCATGATCTCCACGTCGACCGGCCAG
GCCGTCGCGCCGAACGACAACCCGCCCACCACCGGCCCGACGACC
ACCACCACGACGACGTCCCAGCAGCCCGGCGGCTCGTGCACCGCG
ACCTACCGGACCACCCAGCAGTGGGGCGACCGCTTCAACGGCGAG
GTGACCGTCCGGGCGGGCGCCTCCGCGATCACCAGCTGGACGGCC
ACCGTCACGGTGACCTCCCCGCAGAAGGTGTCGGCCACCTGGAAC
GGCACGCCGAGCTGGGACTCCAGCGGCAACGTCATGACCATGAAG
CCCAACGGCAACGGAAACCTCGCCGCGGGCGCGAGCACGACGTTC
GGCTTCACCGTGATGACCAACGGCCAGTGGGCGGCGCCGACCGTC
TCCTGCCGCACGCCGTGA
SEQ 47:
Polynucleotide sequence of exoglucanase
(cex-like) homologue (Cellulomonas palmilytica)
ATGACCCCCTCATCCATGTCCAGACGCGCGCGCTTCGCGGCCGCG
CTCGCAGTCGTCACGCTCGGGGCGACCATCGCTGCGACGATCCCC
GCCCAGGCCGCGGGAAGCACGCTGAAGGATGCCGCAGCGCAGAGC
GGCCGGTACTTCGGCACCGCGATCGCCGGCTTCAAGCTGAGCGAC
TCGACGTACTCGTCCATCGCGAACCGTGAGTTCAACATGATCACG
GCCGAGAACGAGATGAAGATGGACGCGACGGAGCCGTCGCAGAAC
AACTTCAACTTCTCCAGCGGCGACCAGATCCTCAACTGGGCCGTC
CAGAACGGCAAGCGCGTGCGCGGCCACGCCCTGGCCTGGCACTCG
CAGCAGCCCAGCTGGATGCAGGGCATGTCCGGCAGCGCGCTGCGC
AGCGCCATGCTCAACCACGTCACCAAGGTCGCCGAGCACTACAAG
GGCAAGGTCTATGCCTGGGACGTCGTGAACGAGGCGTTCGACGAC
AGCAACGGCGGGCGCCGCGACTCCAACCTGCAGCGCACCGGCAAC
GACTGGATCGAGGCCGCGTTCAAGGCCGCCCGCGCCGCCGACCCG
AACGCCAAGCTCTGCTACAACGACTACAACACCGACAACTGGACC
TGGGCCAAGACGCAGGGCGTCTACAACATGGTCAAGGACTTCAAG
GCCCGTGGCGTGCCGATCGACTGCGTCGGCTTCCAGTCGCACTTC
AACGCGCAGAGCGCCTACAACAGCAACTACCGCACCACGCTGTCG
AGCTTCGCGGCGCTGGGTGTCGAGGTCCAGATCACCGAGCTCGAC
ATCGAGGGCTCCGGCTCGCAGCAGGCGGACACGTACCGTCGCGTC
GTCGAGGACTGCCTCGCCGTCAAGGCCTGCACCGGCATCACGGTG
TGGGGCGTTCGTGACTCCGACTCGTGGCGCTCCTACGGCACGCCG
CTGCTGTTCGACAACAACGGCGGCAAGAAGGCCGCGTACACCTCG
GTCCTCAACGCGCTGAACGCCGCGGACCCGACGGACCCGACGACC
GACCCCACGCAGGACCCGACGGACGACCCGACCCAGGACCCGACG
GACGACCCGACGGACGACCCGACGGACCCGGAGACGACGAACCCG
CCGGACCCGACCGGTAAGTGCTCCGCGGCGCTCACGATCGTGAAC
TCCTGGCCGGGCGGCTACCAGGCCACGGTGACGGTCAAGGCCGGC
TCCTCGTCGATCAACGGCTGGCGGGTCACCCTGCCCAGCAGTGTG
AACACGAACAACCTGTGGAACGGCGTCCTGTCCGGTGGCGTGGTG
ACCAACGCCCCGTACAACGGCTCGGTCGGTGCCGGCCAGTCGACG
ACCTTCGGGTTCGTCGGCAACGGCAGCGCCCCGGGCGCAGGCAAC
CTGACCTGCGCCTGA
SEQ 48:
Polynucleotide sequence of exoglucanase
(cex-like) homologue (Micromonospora
carbonacea)
ATGAGACGCAAGAGAGCCCTCCTGACGACGGTGACCCTCGCGGTC
ACCGGCGCACTCACCGCCGGCGTGCTGGTGACGATGGCCCCCGCC
GCCAGCGCCGGGACGACCCTGCGGGCTGCCGCGGCCGAGAAGGGC
CGCTACTTCGGCGCCGCGGTCGCGACGGGCAAACTCTCCAACAGC
ACGTACACGACGATTCTCAACCGCGAGTTCAACAGCGTCGTGGCC
GAGAACGAGATGAAGTGGGACGCCACCGAGCCGCAGCAGGGCCAG
TTCAACTACAGCGGCGGCGACCGCCTCGTCAGCCACGCCCGGGCC
AACGGGATGAGCGTGCGGGGCCACGCCCTGCTCTGGCACCAGCAG
GAGCCGGGCTGGGCGCAGGGCATGTCCGGCAGCGCCCTGCGCAGC
GCGATGATCAACCACGTCACCCAGGTCGCCACCCACTTCAAGGGG
CAGATCTACGCCTGGGACGTGGTGAACGAGGCGTTCGCCGACGGC
AACAGCGGCGGCCGGCGTGACTCGAACCTCCAGCGCACCGGCAAC
GACTGGATCGAGGCGGCGTTCCGCGCCGCGCGGGCCGCCGACCCG
GGCGCGAAGCTCTGCTACAACGACTACAACACCGACGGGGTCAAC
GCGAAGTCGACCGGCATCTACAACATGGTGCGCGACTTCAAGTCC
CGGGGCGTGCCGATCGACTGCGTCGGCTTCCAGTCCCACCTGGGC
ACCACGCTGGCCAGCGACTACCAGGCCAACCTCCAGCGCTTCGCC
GACCTCGGCGTCGACGTGCAGATCACCGAGCTGGACGTCATGACC
GGCGGCAACCAGGCGAACATCTTCGGCGCGGTGACCCGGGCGTGC
ATGAACGTGTCGCGCTGCACCGGCATCACGGTGTGGGGCGTGCGG
GACTGCGACTCGTGGCGGGGGTCCGACAACGCCCTGCTGTTCGAC
TGCAACGGCAACAAGAAGCCGGCGTACGACTCCGTCCTCAACGCC
CTCAATGCCGGCACCGGCATCCCCAACCCGACGACCACCCCGCCG
AACCCCACCACCACTCCGCCGAACCCGACGACTCCGCCGCCGGGT
GGGGCCGGGTGTTCGGCGACGGTGTCGGCGAATTCGTGGACGGGT
GGTTTCGTGGCCACGGTGAAGGTGACCGCTGGTTCTGGTGGTACC
CGGGGTTGGAACGTGAGTGTGACGTTGCCGGGTGGTACGAGTGTC
ACGGGTACGTGGTCGGCGACGGCCAGTGGTAGTTCGGGGACGGTG
CGGTTCGCCAACGTGGACTACAACGGTCAGCTCGCCGCCGGTCAG
GTGACCGAGTTCGGGTTCCAGGGCAACGGCACCGCGCCCACCCAG
ACCCCCACCTGCACCGCCAGCTGA
SEQ 49:
Polypeptide sequence of exoglucanase
(CEX-like) homologue (Cellulomonas palmilytica)
MTPSSMSRRARFAAALAVVTLGATIAATIPAQAAGSTLKDAAAQS
GRYFGTAIAGFKLSDSTYSSIANREFNMITAENEMKMDATEPSQN
NFNFSSGDQILNWAVQNGKRVRGHALAWHSQQPSWMQGMSGSALR
SAMLNHVTKVAEHYKGKVYAWDVVNEAFDDSNGGRRDSNLQRTGN
DWIEAAFKAARAADPNAKLCYNDYNTDNWTWAKTQGVYNMVKDFK
ARGVPIDCVGFQSHFNAQSAYNSNYRTTLSSFAALGVEVQITELD
IEGSGSQQADTYRRVVEDCLAVKACTGITVWGVRDSDSWRSYGTP
LLFDNNGGKKAAYTSVLNALNAADPTDPTTDPTQDPTDDPTQDPT
DDPTDDPTDPETTNPPDPTGKCSAALTIVNSWPGGYQATVTVKAG
SSSINGWRVTLPSSVNTNNLWNGVLSGGVVTNAPYNGSVGAGQST
TFGFVGNGSAPGAGNLTCA
SEQ 50:
Polypeptide sequence of exoglucanase
(CEX-like) homologue (Promicromonospora
iranensis)
MTRTLTSPRHRRSLRAISLATLTAVVLAGGMAATTAQAAGSTLQA
AATEKGRYFGTAIAANKLSDSTYTTIANREFNMVTAENEMKIDAL
EPNQNQFNWINGDRIVSWARSNGKQVRGHTLAWHSQQPGWMQNMS
GTALRNAMLNHVTQVATHYRGQIHSWDVVNEAFQDGSSGARRDSN
LQRTGNDWIEAAFRAARAADPNAKLCYNDYNTDDWTHAKTQAVYN
LVRDFKARGVPIDCVGLQSHFNAQSPVPSNYQTTISSFAALGVDV
QITELDIEGSGSAQADNYRKVVQACLAVSRCTGISVWGVRDTDSW
RASGTPLLFDGNGNKKPAYTATLDTLNGGTSNPQPGGCTVAVTRS
TDWSDRFNVSLAVSGSSTWRVSIQLQGGQTLQNSWNATVSGSSGT
LTATPNGAGNNFGITVYKNGNTNLPTATCSTT
SEQ 51:
Polypeptide sequence of exoglucanase
(CEX-like) homologue (Micromonospora ferruginea)
MDKVFARTSHSTAARPRTRAAVVSMLAGAAAVAATVAVATSASAG
TTLGASAAEQGRYFGTAVAANKLSDATYVGILNREFNMVTPENEM
KWDATEPSQNQFTYSSSDRIVAHAQANGMRVRGHALAWHSQQPGW
AQSLSGTALRNAMVNHITQVATHFKGKIYAWDVVNEAFDDGNGGR
RDSNLQRTGNDWIEVAFRTARAADPGAKLCYNDYNTDNWTWAKTQ
GVYNMVKDFKARGVPIDCVGFQSHFNSGSPYPGNYRTTLQNFADL
GVDVQITELDIEGSGSAQATTYGNVVKDCLAVARCNGITVWGIRD
SDSWRASGTPLLFDGSGNKKAAYTSTLNALNAGGTTPPTSTPPTS
TPPTSTPPTTPPPTTTPPTTPPPAGACTASLTTNQWQGGFVTTVR
VTAGGSALNGWSVSLTLPSGSSVTNTWSAQASGAGGAVTFRNVDY
NRQVGAGGSTEFGFQGTGTAPSGTVSCTAG
SEQ 52:
Polypeptide sequence of exoglucanase
(CEX-like) homologue (Actinoplanes friuliensis)
MLSAASAGVVLAASIAVVGTAEAASTLGASAAQTGRYYGAAIAAG
RLGDSTYTRILNTEFNSVTPENEMKWDATEPSQGRFTYTNGDRIL
NQGLSNGSKVRGHALLWHAQQPGWAQALSGSALRNAAINHVTQVA
THYKGKIYAWDVVNEAFADGGSGGRRDSNLQRTGNDWIEAAFRAA
RAADPAAKLCYNDYNTDGINAKSTGIFNMVRDFKSRGVPIDCVGF
QSHLGTGIDSTYQANLKRFADLGVDVQITELDIEQGGNQANIYST
VTKACLAVSRCTGITVWGIRDTDSWRTGANPLLFDGSGNKKPAYT
AVLNALNAGGTTNPTTPPVTSPPASPTTPPPSGSGCTATVSLNSW
SGGYVATVKVTAGSSAVTGWTVSATLPSGGALTGVWSATNTGTTG
AVSFRNVEYNGRIPAGGSTEFGFQGTGTGPSAAPACRVG
SEQ 53:
Polypeptide sequence of exoglucanase
(CEX-like) homologue (Actinoplanes ianthinogenes)
MIVAGIAGLVAAGLGVVYTQTADAATTLSASANQTGRYFGTAIPV
SKLGDSAYTTILKTEFNAVTPENEMKWDATEPSQGSFNYTNGDKI
LNQGRAQGAKVRGHALLWHSQQPTWAQSLSGSALRTAAINHVTQV
ATHYKGKIYAWDVVNEAFADGGSGGRRDSNLQRTGNDWIEAAFKA
ARAADPAAKLCYNDYNTDGVNAKSTGVYTMVKDFKARGVPIDCVG
FQSHLGTTVPADYQANLQRFADLGVDVQITELDVAQGGNQANIYA
SVTKACLAVSRCTGITVWGIRDSDSWRTGENPLLFDNSGTKKAAY
TSVLNALNAGGTKPATSATTTPPGTAAPSAGAGCTAKLTAGEVWG
DRYNSTITVSGASTWTVVVTITAPQKVSTVWNGTATYGGGSGQIM
TVKPNGNGNSFGFTTMNNGNSTARPTITSCVAGGSTTSATAAPTT
VPASAPSSCALPSKYRWTSTGALATPKSGWASLKDFTVVPYNGKH
LVYATTHDTGSSWGSMNFSPFTNFSDMASAGQNAMSSGAVAPTLF
YFAPKKIWVLTYQWGGSAFSYRTSSDPTNANGWSAAKPLFTGSIS
GSGTGPIDQTIIGDGTNMYLFFAGDNGKIYRASMPIGNFPGNFGS
SYTTIMSDTTNNLFEAPQVYKVQGQNQYLMIVEAIGSNGRYFRSF
TATSLSGSWTPQAATESNPFAGKANSGATWTNDISHGELVRVTAD
QTMTVDPCHLQLLYQGRSPSSGGDYGLLPYRPGVLTLQR
SEQ 54:
Polypeptide sequence of exoglucanase (CEX-like)
homologue (Antribacter gilvus)
MKDLSPHRAGKFPWRSLAGAAGALAVAASLAVGVSTSAQAAGTTL
QAAAAESGRYFGTAIAANKLSDSTYTTIANREFNMITAENEMKLD
ATEPSQNQFNYTNGDRIVNWATSNGKQVRGHTLAWHSQQPGWMQN
MSGTALRSAMLNHVTQVATHYRGKIHSWDVVNEAFQDGSSGARRD
SNLQRTGNDWIEAAFRAARAADPNAKLCYNDYNTDDWTHAKTQAV
YNLVRDFKARGVPIDCVGLQSHFNSQSPVPSNYQTTLSSFAALGV
DVQITELDIEGSGTAQADNYRRVVQACLAVSRCTGITVWGVRDTD
SWRSSGTPLLFDGSGNKKAAYTSTLNALNAGGTTNPPTTPPPTTP
PPTTPPPTTAPPTTPPPTQPGACTAAYRLVNSWQGGFQAEVVVTA
GSARTGWTTSFTLPGGTSISQLWSGTLTSSGSTHTVRNVSWNGNL
GAGQSTTYGFTGTGSAPSSATVSCS
SEQ 55:
Polypeptide sequence of exoglucanase (CEX-like)
homologue (Nonomuraea jiangxiensis)
MRTNALPPPRTRRSFGGFRRAVAVGVLALAGTIAPLALSVPADAA
ETTLGAAAMQSNRYFGAAIAAGKLNESAYTTIANREFNMVTPENE
MKIDATEPNRGQFTFTNADRIYNWAVQNGKRVRGHTLAWHSQQPG
WMQSLSGSTLRQAMIDHINGVMAHYRGNIYAWDVVNEAFADGNSG
GRRDSNLQRTGNDWIEVAFRTARAADPAAKLCYNDYNIENWTWAK
TQGVYNMVRDFKSRGVPIDCVGLQAHFNSGSPYNSNFRTTLSSFA
ALGVDVQITELDIQGASATTYANVINDCLAVPRCTGITVWGVRDS
DSWRSGDTPLLFDGSGNKKPAYTSVLNALNSVNPNPDTTPPSTPG
TPAASNVTSSGATLTWAASTDTGGSGLAGYNVYREQGTTDPQLGQ
SATNSITLTGLTAGTQYQVYVRARDGAGNLSGNSQLVTFTTQTGG
GTDTTPPSTPGTPAASNVTASGATLTWTASTDTGGSGLAGYDVYR
EQGATDPLLGQSATNSITLTGLTAGTQYQVYVRARDGAGNLSGNS
QPATFTTTGGGTGGSCTVTPTTQTQWPSGYVIDPVRVTAGTSAIS
GWTVTFTLPAGHTVTGSWNTQLTVSGQTVTARNAAHNGNLGPGAS
TAFGFQVSRPNGNTSLPSGYTCA
SEQ 56:
Polypeptide sequence of exoglucanase (CEX-like)
homologue (Kibdelosporangium aridum)
MSRTVGRHAALLALLGTFLLPGTADAGTTLGASAAEKGRYFGAAV
AAHKLGDSTYVGILNREFNMVTPENEMKIDATEPNQNQFSFGNAD
RIVNHAVSQGMRVRGHTLAWHSQQPGWMQNMSGSALRQAMLNHVT
RVASYYRGKIYAWDVVNEAFADGSSGARRDSNLQRTGNDWIEAAF
RAARAADPNAKLCYNDYNTDDWSHAKTQAVYRMVQDFKSRGVPID
CVGLQSHFNNNSPYPSNYRTTLSSFAALGVDVQITELDIEGAPPT
TYGNVVRDCLAVARCNGITVWGIRDSDSWRASQTPLLFNNSGGKK
PAYDAVLSALNSGSIPPPGGSNCSAGYVGLTFDDGPNSSTTPQLL
NALRSAGVRATFFNVGQRVQQNPALTRSQIDAGMWVGNHSWTHPH
LTQMTAAQITSELSQTQQALQQATGQTPRLFRPPYGETNSTVRQV
QAQLGLTEVMWTVDSQDWNNASTAQIVQAASTLQPGGIILMHDGY
QTTVNAIPQIVANLSSRNLCAGMISTNGQVVAPGTEPGNGSCTAS
YRTTQQWGDRENGEVTIKAGSAAITSWTSTVTITAPQRVSTTWNG
TASWDSSGTVMTMKPNGNGNLAAGASTTFGFTVMANGQWAQPSVS
CGSP
SEQ 57:
Polypeptide sequence of exoglucanase (CEX-like)
homologue (Actinomadura madurae)
MRANVIPKSRIRIRKRAILAGALGVLATAALVAPSPAAAAESTLG
AAAAQNGRYFGTAIASGKLNDSVYTTIANREFNSVTAENEMKIDA
TEPQRGRFDFSAGDRVYNWAVQNGKQVRGHTLAWHSQQPGWMQSL
EGGTLRQAMIDHINGVMAHYTGKIVQWDVVNEAFADGSSGARRDS
NLQRTGNDWIEVAFRTARAADPAAKLCYNDYNIENWTWAKTQAVY
SMVRDFKQRGVPIDCVGFQSHFNSGSPYNGNFRTTLQSFAALGVD
VAITELDIQGASATTYANVINDCLAVSRCLGITVWGVRDTDSWRS
EQTPLLFDGNGNKKAAYTAVLNALNGGTTTPPDGAGTIKGDGSGR
CLDVPNASTTDGTQVQLWDCHGGTNQQWTYTDASELRVYGDKCLD
AAGTGNGARVQIYSCWGGDNQKWRLNSDGSVVGVQSGLCLDAAGT
ANGSAIQLYSCWNGGNQRWTRT
SEQ 58:
Polypeptide sequence of exoglucanase (CEX-like)
homologue (Nonomuraea indica)
MRVDAMSPPTARRPLGARRRTLITGVLGALGLITALAPAIPADAA
ASTLGAAAAQSGRYFGTAIASGKLGDSAYTTIAGREFDMVTAENE
MKIDATEPNRGQFTFTAGDRVYNWAVQNGKRVRGHTLAWHNQQPG
WMQSLSGSSLRQAMINHINGVMGHYKGKIYAWDVVNEAYADGNSG
GRRDSNLQRTGNDWIEVAFRTARAADPAAKLCYNDYNIDNWTWAK
TQGVYNMVRDFRARGVPIDCVGLQSHFNANSPYNSNYRTTIQSFA
ALGVDVQITELDIQGGSATTYANVVRDCLAVPRCTGISVWGVRDS
DSWLGAGATPLLFDGNGNKKAAYTAVLDVLNGGSTTPPPGDAGQI
RNTASGRCVDVPNSATADGTAVQLWDCNGQSNQRWTRTAAGELKY
GDKCLDAGGTGNGARIQIYSCWGGDNQKWRLNSDGTIVGVQSGLC
LDAVGGGTGNGTGLQLYGCWGGGNQRWDYNPGTA
SEQ 59:
Polypeptide sequence of exoglucanase (CEX-like)
homologue (Saccharothrix syringae)
MSRTAVTAAGRSEARGRSRRAAVVVGAIGLLSAAAVVLPNVATAG
TTLGASAAESGRYFGTAVAANKLSDSTYVGILNREFDMVTAENEM
KMDATEPNQNQFSFGNGDRIVNHARNQGKRVRGHALAWHSQQPGW
MQNMSGTALRNAMLNHVTQVATYYKGKIYAWDVVNEAYADGSSGG
RRDSNLQRTGNDWIEAAFRAARAADPNAKLCYNDYNTDNWSHAKT
QGVYRMVQDFKSRGVPIDCVGFQAHFNSGNPVPSNYHTTLQNFAD
LGVDVQITELDIEGSGSTQAQQYQGVVQACLAVTRCTGITVWGIR
DSDSWRSSGTPLLFDGSGNKKAAYTSVLNALNAGSTAPPSTTTTT
TPPNTSDCRAGYVGLTYDDGPNGSTTTQLLNALRSAGLRATFFNQ
GNRVQQNPGLAKAQRDAGMWVGNHSWSHPHMTQLSQSQMASEISQ
TQQAIQSATGEAPKLFRPPYGETNSTLKSVEAQYGLTEVLWSVDS
QDWNNASTAQIVQAASTLQNGGVILMHDGYQTTINAIPQIAANLA
SRGLCAGMISTSTGQAVAPNDNPPTTGPTTTTTTTSQQPGGSCTA
TYRTTQQWGDRENGEVTVRAGASAITSWTATVTVTSPQKVSATWN
GTPSWDSSGNVMTMKPNGNGNLAAGASTTFGFTVMTNGQWAAPTV
SCRTP
SEQ 60:
Polypeptide sequence of exoglucanase (CEX-like)
homologue (Cellulomonas cellasea)
MNRSGLRPPSSTHRRRTGAVLVAALAVTLAGATLPAQGAGSTLQA
AAAESGRYYGTAIAANKLSDSTYTTIANREFNMITAENEMKMDAT
EPSQNQFNYSSGDRIVSWARSNGKRVRGHALAWHSQQPGWMQNMS
GTALRNAMLNHVTQVATHYKGQIYAWDVVNEAYADGSSGARRDSN
LQRTGNDWIEAAFRAARAADPAAKLCYNDYNTDNWSHAKTQGVYT
MVRDFKSRGVPIDCVGFQAHFNSGNPVPSNYDVTLRNFAALGVDV
QITELDIEGSGSSQAQQYAGVHQACLSVARCTGVTVWGVRDTDSW
RASGTPLLFDGSGNKKAAYTSTLNALNAGGTTTPNPTPNPTTPQP
TPTTTTPPVTGTGSCTATYSEGQKWGDRFNGVVTIRANSAITSWT
STVTVSQAQRITSTWSGTPSWDSSGKVMTMRPAGNGTLAAGQTTS
FGFTVLHGGDWTWPRVTCSAS
SEQ 61:
Polypeptide sequence of exoglucanase (CEX-like)
homologue (Couchioplanes caeruleus)
MHKPRTPLRALGVAAAAAVVAAGSVVALTSTAEAATTLGASAAAT
GRYFGTAVAANKLSDSTYVGILDREFNAVTPENEMKWDATEPNQN
QFNYSSADRIVSHAQAQNMRIRGHALAWHQQQPGWAQNLSGTALR
NAMLNHVTTVAAHYKGQIYAWDVVNEAFDDGGNGARRDSNLQRTG
NDWIEAAFRAARAADPGAKLCYNDYNTDGQNAKSNAVYAMVQDFK
SRGVPIDCVGFQSHFNAQSPVPSDYQANLQRFANLGVDVQITELD
IEGSGQTQADNFGRVVKACLAVSRCTGITVWGIRDSDSWRASGTP
LLFDSSGNKKAAYTSTLNALNSGGTTPPSDPPTSPSTPPSTPTDP
GTGACTATISLNQWNGGFVATVRVSAGSSALSGWSVSTTLPSGAA
VTNSWSSQSSGSSGTVRFANVSYNGSVAAGGSTEFGFQGTGTGPS
SATCSAS
SEQ 62:
Polypeptide sequence of exoglucanase (CEX-like)
homologue (Glycomyces tritici)
MITSTPRRRRRVWSTIAAVATASLAATTALVMLPGTAQAGTTLGA
SAAESGRYFGAAVSTQYLSESAYANTLGAEFNSVVAENAMKWDAT
EPNPGQFNFSGGDQLVNWAQARGMKVRGHTLVWHAQQPGWAQNLT
GQNLRNAMLNHIAGVANHYEGDVFAWDVVNEAFEWDGSRRQSNLQ
RQLGNGWIEEAFRAADAADPTATLCYNDYGTDSINAKSTAIYNMV
RDFKSRGVPIDCVGFQTHIAHNENLSSYQANLQRFADLGVDVEIT
ELDVGQGSGQAATYGTVTNACMAVARCKGITVWGITDKYSWRDDN
PLLFDGNYQKKQAYHTVLAALNNGNPGGGGGKHITSAASGRCLDV
PNQSTTNGTQLQIYDCWTGANQQWTVANGEISVYSGGSKKCLDAS
GGGTANGTAAIIWSCHGGANQRWNVNSNGTITNAASGLCLDVAAA
ATANGSKVQLWSCTGGSNQRWTV
SEQ 63:
Polypeptide sequence of exoglucanase (CEX-like)
homologue (Actinoplanes auranticolor)
MTVIAARARLLLGAACAVVIAVSSVAVATLAHADLTGPPGTTLKA
AAERSGRYFGAAMGRDRLTDNGFLTIANREFDMMTAVNEMKPDAT
EPNRGQFDFRAGDAIYNWATQRGMRFRGHTLAWHAQQPRFWGSLS
GSALRQAMIDHINGVMAHYKGKLYAWDVVNEAFAENGSRRSSNLQ
ATGNDWIEAAFRAARAADPGVQLCYNDYNIENWTYAKTQGVYNMI
RDFKARGVPIDCVGLQTHFTGGSSLPGNFQQTLSSFAALGVDVAL
TEADVTNASSSQYQGLTQACMNVPRCVGITTWGIRDSDSWRGNEN
PLLFDRNSNPKPAYTSVLNALNAASTTVPGTSTPPSTPPSTPPST
PPSTPPSTPPVTGQPGACTATYRTASTWNGGYQGEVTVANNGSAA
LTGWTVQLTLAGGQTVANVWNGINTGTSGTISVRNAAYNGSVGAN
ASTSFGFLVNGSTGTAPGSLTCTSP
SEQ 64:
Polypeptide sequence of exoglucanase (CEX-like)
homologue (Sphaerisporangium rubeum)
MVVIGTAVVVGAGVCAGAGAADAAGTLGAAAAGSGRDFGTAVQAG
RLGEAAYVETLDREFTSVTPENEMKWDAVEPARGTFVFTAADRIV
EHARARGMKIRGHTLVWHPGLPGWLTNLSSAEFRIAVNNHIATVM
GHWRGQIDSWDVVNEAFQDGSGALRSTIFRSRLGDGYIEEAFRAA
RAADPAAKLCYNDYNIEDADAAKTRAVYAMVRDFKERGVPIDCVG
VQSHFNASMPFPANYRRTIEQFAALGVDVQITQLDVDGGAAQAAT
YRAAVEACVAVPRCTGITVWGVPDHYSWRAPNTPLLFDRDYQKKP
AYFAVLEALNAAGGEPTPAPTPTPSPTPTPTPTVTPVPVSCRVTA
ELWARSRTGYVIKPVTVRNTGRAAVSGWTVTFTLPQGHVVTGSWD
AVLTVAGGTVTARNAGHNGTLAPGATATFGFQVRRASGGTALPSG
YACRAG
SEQ 65:
Polypeptide sequence of exoglucanase (CEX-like)
homologue (Phytoactinopolyspora
halotolerans)
MITRHRRQAILASMALVGATLVVPIGAAQAQDTLRGAADAQGLRM
GAAVANGPLSSDAQYRNILGTEFNSVTAENSMKWESLQPSQGQFT
FSQGDAIVDFAQANNQAVYGHTLVWHNQTPSWVQNLSGQQLDDAM
QTHITTVLDHYEGQVEAWDVANEVIDDGANLRNSFWLQGLGEGYI
ADAFRYADAADPNAKLYINDYNIDGINAKSNAYYDLVSGLLNQGV
PIDGIGLQAHMILGQVPSSLEDNIRRFAQLGLEVRITELDIRMDL
PVTQAKLEQQRQDYAAVVDACMSVDGCVGVTTWGFTDAHSWVPDQ
FGGQGAALPFDENYNKKPAYYGILDTLDGGTPNDTTPPTQPGAPQ
ISDVTSNSASLTWSASSDSGGSGLAGYSVYREQSGNDQLLASPST
NSVTLTGLDPQTQYSVYVVARDGAGNTSSPSTAASFTTQEGPGGG
GACDVEYTANNWGGSEGFTASVTITNTGSSQLNGWTLGFTFPGDQ
SVREGWSATWSQSGADVTAESIGWNDALAPGGSTTVGFNGTYSGS
NPEPTEFTLNGEPCSVS
SEQ 66:
Polypeptide sequence of exoglucanase (CEX-like)
homologue (Glycomyces paridis)
MHTRKRRRALIAGVVTVATAALGIGLSTQFASAQTTLRGAADEAG
IDIGIAVDANQLQNNATYRNLVATEFNSLTAENAMKWDATEPSDN
SWNFSGADAIVDFAEDNDQTVHGHTFVWHSQTPQWVQNLSASAMQ
AAMVDHINTLANRYEGRVDSWDVVNEVVSDNNGAMRDSFWRNTLG
DGYITTAFQTARAADPNADLYINDYSIEGDNAKSDRIYTIAQGLN
SQNLIDGVGFQSHLILGQVPSTMEANLQRFIDLGLKVRITELDIR
IQTPADANELQQQANDYERVVSICAELADCSGVTVWGLRDADSWV
PGVFPGYGAPLLFDDNFGKKPAYNAVLEALGGDGSEEPTTTDPGT
TTPPPTGDGDCTAEIDVVNDWGSGWQGNVLITADNGAVNGWTLTW
TWPSGQSITSSWNATVTSSGSSVTASDVGWNANIAAGQTMNAWGF
VGSGSSAAPQVTCTAD
SEQ 67:
Polypeptide sequence of exoglucanase (CEX-like)
homologue (Streptomonospora alba)
MRLVRHGGHETPRRTRAGSARRVAGAALATFLGAAVLAPAGPAAA
DSPLRDHAAQQGFEIGAALGVDHLQNDSQFADLAATEFNAATAEN
SMKWESTEPSRGQFDFSGADAFMDFAQQNNQKVRGHTLVWHSQLP
SWVENGNFSQSELRSVMEDHIDEVAGRYAGEVAYWDVANEIFEGD
GSWRNSVFYDTLGPDFVADALRMTAEADPNAELWLNDYSIDGINA
KSDAYYNLIQDLQAQGVPIDGIGLQAHLINGQVPGDLQQNIQRFA
DLGIKVAITELDVRIDMPASQSELQQQAQDYRAVMDACLAVNGCV
GVTVWGIVDKYSWVPDTFEGEGAPLLFNDDYQPKPAYDAVHEALG
GSSDDDDDDDNGGGEDPPPTGPCEVSYSVANEWNSGFTGQVTVTN
GGSSALNGWDLQFDFSGGQQITNGWNADWSQSGSTVTASNTEWNG
SVPAGGSVDIGFNASHSGSNPEPGSFALNGESCSVS
SEQ 68:
Zymomonas mobilis codon optimized polynucleotide
sequence of exoglucanase (cex-like)
ATGACGCCAAGTTCAATGTCCCGTCGGGCCCGCGTTGCGTCAGCT
CTTGCCATCGTGACGTTGGGCGCTACTATCGCTACTACGATTCCT
GCCCAAGCCGCCGGCTCTACATTACAAGCGGCTGCTTCTGAAAGT
GGTCGTTATTTCGGTACAGCGATTGCTGCGTTTAAATTGAATGAT
TCAACGTACAGCTCCATTGCGAATCGGGAGTTCAATATGATTACA
GCAGAAAATGAAATGAAAATGGACGCGACTGAGCCGTCTCAGAAT
AATTTTTCGTATTCGAGTGGCGATCAGATTCTTAATTGGGCACGG
TCTAATGGTAAGCGTGTTCGTGGCCATGCATTGGCCTGGCATTCG
CAGCAACCGGGGTGGATGCAAAATATGTCCGGTACACAGTTACGC
AATGCCATGTTGAATCATGTGACGCAGGTTGCTACGCATTATAAA
GGCAAGATTTATGCATGGGATGTCGTTAATGAAGCCTATGCGGAC
TCTGGTGGGGGGCGTCGTGACAGTAATCTTCAACGGACCGGTGAT
GATTGGATTGAAGCTGCTTTTCGCGCTGCGCGGGCGGCAGATCCA
GGTGCCAAACTTTGCTATAATGATTATAATACAGACAATTGGACG
TGGGCTAAAACACAGGGTGTCTACAACATGGTTAAAGATTTTAAA
GCTCGTGGGGTGCCGATTGATTGCGTTGGCTTTCAATCTCATTTC
AATTCCGGGTCGCCGTATCCGTCAAATTATCGTACAACCTTACAA
AATTTCGCCGCCCTGGGTGTGGAAGTTCAGATTACCGAATTAGAT
ATCGAAGGTTCGGGGCAACAACAGGCACAGACTTATGCCAATGTC
GTCGCTGATTGCTTGGCAGTGAAGGCTTGCACCGGTATCACAGTT
TGGGGCGTCAGAGACTCAGACTCTTGGCGCAGCTCCGGGACGCCG
TTGTTGTTTGATGGCAGCGGTAATAAAAAGGCTGCGTATACCTCT
ACCTTGGACGCTCTTAACCGGGGTGGAGTCCCGACTGATCCGACC
ACTCCGCCGACAGACCCCACGACACCACCCACTGATCCCACGACA
CCGCCTACCGATCCTACCACTCCGCCTACGGATCCTACAACCCCA
CCCACGGATCCAACTGGCCGTTGCACCGCGAGCCTTGCGATTGCC
AACGCCTGGCCGGGTGGATATCAGGCCACGGTCACGGTCAAAGCA
GGGTCCAGTAGTATCAATGGATGGCGCGTTACATTACCTTCTGGC
GTTTCCACTAGTAACCTGTGGAATGGCGTCCTGGCGAACGGAGTG
GTCACGAATGCCCCTTATAATGGCTCTGTTGGCGCGGGGCAATCC
ACGACCTTTGGCTTTGTCGGCAATGGCTCGGCGCCCAGTGCTGGT
AGCGTGACTTGTGCCTGA

1. A genetically modified ethanologenic organism which comprises an exoglucanase (cex-like) polynucleotide sequence with at least 70% sequence coverage to SEQ 1 or SEQ 68, and at least 70% sequence identity to SEQ 1 or SEQ 68.

2. The genetically modified ethanologenic organism according to claim 1, wherein said cex-like polynucleotide sequence is selected from the group consisting of SEQ 1; SEQ 41; SEQ 42; SEQ 43; SEQ 44; SEQ 45; SEQ 46; SEQ 47; SEQ 48; and SEQ 68.

3. The genetically modified ethanologenic organism according to claim 1, wherein said cex-like polynucleotide sequence, upon transcription and translation under the control of a native or synthetic promoter and Ribosomal binding site (RBS), provides an exoglucanase (CEX-like) polypeptide sequence, wherein said CEX-like polypeptide sequence has at least 70% sequence coverage to SEQ 2, and at least 35% sequence identity to SEQ 2.

4. The genetically modified ethanologenic organism according to claim 3, wherein said CEX-like polypeptide sequence is selected from a group consisting of SEQ 2; SEQ 49; SEQ 50; SEQ 51; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; and SEQ 67.

5. A genetically modified ethanologenic organism which comprises a (3-glucosidase 1 (bgl1) polynucleotide sequence with at least 70% sequence coverage to SEQ 3 or SEQ 14, and at least 70% sequence identity to SEQ 3 or SEQ 14.

6. The genetically modified ethanologenic organism according to claim 5, wherein said bgl1 polynucleotide sequence is selected from the group consisting of SEQ 3; SEQ 14; SEQ 15; SEQ 16; SEQ 17; SEQ 18; SEQ 19; SEQ 20; and SEQ 21.

7. The genetically modified ethanologenic organism according to claim 5, wherein said bgl1 polynucleotide sequence, upon transcription and translation under the control of the native of a native or synthetic promoter and Ribosomal binding site (RBS), provides a 3-glucosidase 1 (BGL1) polypeptide sequence, wherein said BGL1 polypeptide sequence has at least 70% sequence coverage to SEQ 4, and at least 35% sequence identity to SEQ 4.

8. The genetically modified ethanologenic organism according to claim 7, wherein said BGL1 polypeptide sequence is selected from a group consisting of: SEQ 4; SEQ 22; SEQ 23; SEQ 24; SEQ 25; SEQ 26; SEQ 27; SEQ 28; SEQ 29; SEQ 30; SEQ 31; SEQ 32; SEQ 33; SEQ 34; SEQ 35; SEQ 36; SEQ 37; SEQ 38; SEQ 39; and SEQ 40.

9. A genetically modified ethanologenic organism which comprises:
a. an exoglucanase (cex-like) polynucleotide sequence with at least 70% sequence coverage to SEQ 1 or SEQ 68, and at least 70% sequence identity to SEQ 1 or SEQ 68; and
b. a β-glucosidase 1 (bgl1) polynucleotide sequence with at least 70% sequence coverage to SEQ 3 or SEQ 14, and at least 70% sequence identity to SEQ 3 or SEQ 14.

10. The genetically modified ethanologenic organism according to claim 9, which comprises:
a. an exoglucanase (cex-like) polynucleotide sequence selected from the group consisting of: SEQ 1; SEQ 41; SEQ 42; SEQ 43; SEQ 44; SEQ 45; SEQ 46; SEQ 47; SEQ 48; and SEQ 68; and
b. a β-glucosidase 1 (bgl1) polynucleotide sequence selected from the group consisting of: SEQ 3; SEQ 14; SEQ 15; SEQ 16; SEQ 17; SEQ 18; SEQ 19; SEQ 20; and SEQ 21.

11. The genetically modified ethanologenic organism according to claim 9, which upon transcription and translation under the control of the native of a native or synthetic promoter and Ribosomal binding site (RBS), comprises:
a. an exoglucanase (CEX-like) polypeptide sequence, wherein said CEX-like polypeptide sequence has at least 70% sequence coverage to SEQ 2, and at least 35% sequence identity to SEQ 2; and
b. a β-glucosidase 1 (BGL1) polypeptide sequence, wherein said BGL1 polypeptide sequence has at least 70% sequence coverage to SEQ 4, and at least 35% sequence identity to SEQ 4.

12. The genetically modified ethanologenic organism according to claim 11, wherein:
a. said exoglucanase (CEX-like) polypeptide sequence is selected from the group consisting of: SEQ 2; SEQ 49; SEQ 50; SEQ 51; SEQ 52; SEQ 53; SEQ 54; SEQ 55; SEQ 56; SEQ 57; SEQ 58; SEQ 59; SEQ 60; SEQ 61; SEQ 62; SEQ 63; SEQ 64; SEQ 65; SEQ 66; and SEQ 67; and
b. said β-glucosidase 1 (BGL1) polypeptide sequence is selected from the group consisting of: SEQ 4; SEQ 22; SEQ 23; SEQ 24; SEQ 25; SEQ 26; SEQ 27; SEQ 28; SEQ 29; SEQ 30; SEQ 31; SEQ 32; SEQ 33; SEQ 34; SEQ 35; SEQ 36; SEQ 37; SEQ 38; SEQ 39; and SEQ 40.

13. The genetically modified ethanologenic organism according to claim 1 further comprising a promoter and RBS sequence which drives gene expression, wherein the genetic material for the promoter/RBS sequences comprises at least one or another of the following: SEQ 5; SEQ 6; SEQ 7; SEQ 8; SEQ 9; SEQ 10; SEQ 11; SEQ 12; and SEQ 13.

14. The genetically modified ethanologenic organism according to claim 1, where the organism is a bacterium.

15. The genetically modified ethanologenic organism according to claim 1 where the organism is selected from the group consisting of the genera Aspergillus, Mucor, Zymomonas, Escherichia, Clostridia, Bacillus, and Pseudomonas.

16. The genetically modified ethanologenic organism according to claim 1 where the organism is a prokaryotic organism.

17. The genetically modified ethanologenic organism according to claim 1 where the organism belongs to the bacterial genus Zymomonas.


SEQUENCE LISTING (68 sequences)

SEQ ID NO: 1 - DNA, 1413 bp, Cellulomonas uda
  source 1..1413: mol_type = genomic DNA
atgaccccctcatccatgtccagacgcgcgcgcgtcgcgtccgcgctcgcgatcgtcacgctcggcgcgaccatcgcgacgacgatccccgcccaggccgccggcagcacgctgcaggccgcggcctccgagagcggccggtacttcggtaccgccatcgcggcgttcaagctcaacgacagcacgtactcgtccatcgcgaaccgtgagttcaacatgatcacggccgagaacgagatgaagatggacgcgacggagccgtcgcagaacaacttcagctactccagcggcgaccagatcctcaactgggcgcgcagcaacggcaagcgggttcgtgggcacgcgctcgcgtggcactcgcagcagccgggctggatgcagaacatgtccggcacccagctgcgcaacgcgatgctcaaccacgtcactcaggtcgcgacgcactacaagggcaagatctacgcctgggacgtcgtgaacgaggcgtacgcggacagcggcggcggccgtcgcgactcgaacctgcagcgcaccggcgacgactggatcgaggcggcgttccgcgcggcccgcgccgcggacccgggcgccaagctctgctacaacgactacaacacggacaactggacctgggccaagacgcagggcgtctacaacatggtcaaggacttcaaggcccgtggggtgccgatcgactgcgtcggtttccagtcacacttcaactcgggcagcccgtacccgagcaactaccggaccacgctgcagaacttcgccgcgctcggcgtcgaggtgcagatcaccgagctcgacatcgagggctcgggccagcagcaggcccagacgtacgccaacgtggtcgccgactgcctcgccgtgaaggcctgcaccggcatcacggtgtggggcgtgcgcgactccgactcgtggcgctcctcgggtaccccgctgctgttcgacggctcgggcaacaagaaggccgcgtacacctccacgctcgacgcgctgaaccgcggcggcgtcccgacggacccgacgacgccgcccacggacccgaccacgccgcccacggacccgacgaccccgcccacggacccgaccaccccgccgaccgaccccaccacgcctccgaccgacccgacgggccggtgcacggcgagcctggcgatcgcgaacgcgtggcccggcgggtaccaggcgaccgtgaccgtcaaggcgggctcgtcgtcgatcaacggctggcgcgtcacgctgcccagcggtgtcagcacgagcaacctctggaacggcgtgctcgccaacggtgtggtgaccaacgcgccgtacaacggctcggtcggcgccggccagtcgacgaccttcgggttcgtcggcaacggcagcgctccgagcgcaggctccgtgacctgcgcctga

SEQ ID NO: 2 - protein, 470 aa, Cellulomonas uda
MTPSSMSRRARVASALAIVTLGATIATTIPAQAAGSTLQAAASESGRYFGTAIAAFKLNDSTYSSIANREFNMITAENEMKMDATEPSQNNFSYSSGDQILNWARSNGKRVRGHALAWHSQQPGWMQNMSGTQLRNAMLNHVTQVATHYKGKIYAWDVVNEAYADSGGGRRDSNLQRTGDDWIEAAFRAARAADPGAKLCYNDYNTDNWTWAKTQGVYNMVKDFKARGVPIDCVGFQSHFNSGSPYPSNYRTTLQNFAALGVEVQITELDIEGSGQQQAQTYANVVADCLAVKACTGITVWGVRDSDSWRSSGTPLLFDGSGNKKAAYTSTLDALNRGGVPTDPTTPPTDPTTPPTDPTTPPTDPTTPPTDPTTPPTDPTGRCTASLAIANAWPGGYQATVTVKAGSSSINGWRVTLPSGVSTSNLWNGVLANGVVTNAPYNGSVGAGQSTTFGFVGNGSAPSAGSVTCA

SEQ ID NO: 3 - DNA, 2643 bp, Aspergillus niger
  source 1..2643: mol_type = genomic DNA
atgaggttcactttgatcgaggcggtggctctgactgccgtctcgctggccagcgctgatgaattggcctactcccctccgtattacccctccccttgggccaatggccagggtgactgggcggaagcataccagcgcgctgttgatatcgtctcgcagatgacattggctgagaaggtcaatttgactacgggaactggatgggaattggaattatgtgttggtcagactggaggtgttccccgattgggaattccgggaatgtgtgcacaggatagccctctgggtgttcgtgactccgactacaactctgcgttccccgccggtgtcaacgtggccgcaacctgggacaagaatctggcttacctgcgtggccaggctatgggtcaggagtttagtgacaagggtgctgatatccaattgggtccagctgccggccctctcggtagaagtcccgacggcggtcgtaactgggagggcttctcccccgacccggccctcagtggtgtgctctttgcagagacaatcaagggtattcaggatgctggtgtggttgcaacggctaagcactacatcgcctacgagcaggagcatttccgtcaggcgcctgaagctcaaggctacggattcaatattaccgagagtggaagcgcgaacctcgacgataagactatgcatgagctgtacctctggcccttcgcggatgccatccgtgcaggtgccggtgctgtgatgtgctcgtacaaccagatcaacaacagctatggctgccagaacagctacactctgaacaagctgctcaaggctgagctgggtttccagggctttgtcatgagtgattgggcggctcaccatgccggtgtgagtggtgctttggcgggattggacatgtctatgccgggagacgtcgattacgacagtggcacgtcttactggggtaccaacttgaccatcagtgtgctcaacgggacggtgccccaatggcgtgttgatgacatggctgtccgcatcatggccgcctactacaaggtcggccgtgaccgtctgtggactcctcccaacttcagctcatggaccagagatgaatacggcttcaagtactactatgtctcggagggaccgtatgagaaggtcaaccagttcgtgaatgtgcaacgcaaccatagcgagttgatccgccgtattggagcagacagcacggtgctcctcaagaacgatggcgctcttcccttgactggaaaggagcgcttggtcgcccttatcggagaagatgcgggttccaatccttatggtgccaacggctgcagtgaccgtgggtgcgacaatggaacattggcgatgggctggggaagtggcactgccaactttccctacttggtgacccccgagcaggccatctcgaacgaggtgctcaagaacaagaatggcgtattcactgcgaccgataactgggctattgatcagattgaggcgcttgctaagaccgccagtgtctctcttgtctttgtcaacgccgactctggtgagggttatatcaatgtcgacggaaacctgggtgaccgcaggaacctgaccctgtggaggaacggcgacaatgtgatcaaggctgctgctagcaactgcaacaacacgatcgttattattcactctgtcggcccagtcttggttaacgagtggtacgacaaccccaatgttaccgctattctctggggtggtcttcccggtcaggagtctggcaactccctcgccgacgtgctctacggccgtgtcaaccccggtgccaagtcgcccttcacctggggcaagactcgtgaggcctaccaagattacttgtacaccgagcccaacaacggcaacggagcgccccaggaagacttcgtcgagggcgtcttcattgactaccgcggatttgacaagcgcaacgagactcctatctatgagttcggctatggtctgagctacaccaccttcaactactcgaaccttcaggtggaggttctgagcgcccctgcgtacgagcctgcttcgggcgagactgaggcagcgccgactttcggagaggtcggaaatgcgtcggattacctctaccccgatggactgcagagaatcaccaagttcatctacccctggctcaacagtaccgatcttgaggcgtcttctggggatgctagctatgggcaggatgcctcagactatcttcccgagggagccaccgatggctctgcgcaaccgatcctgcctgccggtggtggtgctggcggcaaccctcgcctgtacgacgagctcatccgcgtgacggtgactatcaagaacaccggcaaggttgcgggtgatgaagttcctcaactggtaagtagacagcaaattcgaaccaagtcggaccaagctaatgaatcgcagtatgtttctcttggcggccctaacgaacccaagatcgtgctgcgtcaattcgagcgtatcacgctgcagccgtcggaagagacgcagtggagcacgactctgacgcgccgtgaccttgcgaactggaatgttgagacgcaggactgggagattacgtcgtatcccaagatggtgtttgtcggaagctcctcgcggaagctgccgctccgggcgtctctgcctactgttcactaagaattcgcc

SEQ ID NO: 4 - protein, 877 aa, Aspergillus niger
MRFTLIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVTVTIKNTGKVAGDEVPQLVSRQQIRTKSDQANESQYVSLGGPNEPKIVLRQFERITLQPSEETQWSTTLTRRDLANWNVETQDWEITSYPKMVFVGSSSRKLPLRASLPTVH

SEQ ID NO: 5 - DNA, 250 bp, Zymomonas mobilis
  source 1..250: mol_type = genomic DNA
tcgacaattttacgcgtttcgatcgaagcagggacgacaattggctgggaacggtatactggaataaatggtcttcgttatggtattgatgtttttggtgcatcggccccggcgaatgatctatatgctcatttcggcttgaccgcagtcggcatcacgaacaaggtgttggccgcgatcgccggtaagtcggcacgttaaaaaatagctatggaatatagtagctacttaataagttaggagaataaac

SEQ ID NO: 6 - DNA, 250 bp, Zymomonas mobilis
  source 1..250: mol_type = genomic DNA
ataatcacttaatccagaaacgggcgtttagctttgtccatcatggttgtttatcgctcatgatcgcggcatgttctgatatttttcctctaaaaaagataaaaagtcttttcgcttcggcagaagaggttcatcatgaacaaaaattcggcatttttaaaaatgcctatagctaaatccggaacgacactttagaggtttctgggtcatcctgattcagacatagtgttttgaatatatggagtaagca

SEQ ID NO: 7 - DNA, 250 bp, Zymomonas mobilis
  source 1..250: mol_type = genomic DNA
gcgatggtgccgctggctaacatgttcggctatgtgaaccagctccgttccttcacccagggacgcgcccagtattcgatgcaattttcgcattatgacgaagtcccggctaacgtcgcggatgagttaaaatcgaagatggcttaattaaatactggcataaaccgaaaaatgtcgttatgagcgcgccggagaagcgcggcgcgctcaatacaatagtgataaaagcggtaacaaaaagaggtaacta

SEQ ID NO: 8 - DNA, 250 bp, Zymomonas mobilis
  source 1..250: mol_type = genomic DNA
aatcattggcagctgatgctacattcgccacagttttgttttcggccattgtctatactccagttactcaatacgtaacaataatcagtttatcctaactatagaatcgcatgagaagcgataacgtttcaccataagcaatatattcattgcaacagtggaattgccttatgcgtcaaggaaggatagatcattgacggactgagttcaaaaagagactcgtctaaaagattttaagaaaggtttcgat

SEQ ID NO: 9 - DNA, 250 bp, Zymomonas mobilis
  source 1..250: mol_type = genomic DNA
actatagaatataagttatgttccattcgcagaatagatatagatcagcctctatggatatgctatatatcgcccattccatttaagaataataataaaccatcatgctgtttatttaatatttttattacagtgaattgaagaaatattttcttgataaaaattattaaaaatctatcaccgacgatccgtctctatttcaagatagataataatttgtttaacctgttgattatgcgagataatttta

SEQ ID NO: 10 - DNA, 250 bp, Zymomonas mobilis
  source 1..250: mol_type = genomic DNA
gcagcattaagtatcttaggtggcttgattgttgctcgcttcgtgccggaaaccaaaggtcggagcctggatgaaatcgaggagatgtggcgctcccagaagtagttaaacttgctttggctgaatccttttgtcttttttagataagtcttaaccaattatactttttgtttacaacgatggtataaagcgggcggacaggctaaaaacaggctaaaaggattcggcctctgttttaaggacgagaata

SEQ ID NO: 11 - DNA, 250 bp, Zymomonas mobilis
  source 1..250: mol_type = genomic DNA
ttaaaaaataacttcattttactttaaattttccaagaaaatatttcgaaaatatttttgatatctttcttaattaagaaagaaaacttagttataatcctaccagttggacgaatcgcagacggtcgatttcgatttattcaaaaggccttttggcacagaagaaaaatcgaggtcatcgtcataatttaaagcgaatggacagcatatacctccgtattacggggggattttgtgagtggtgagaata

SEQ ID NO: 12 - DNA, 250 bp, Zymomonas mobilis
  source 1..250: mol_type = genomic DNA
aggcaccaacgcatcaagaacatcagccgccaatcggcctgctttccgcattccggcaaaagcgttttcatcgtaaagtttgatggcatgcgtacgaatagccgcatcaccttctttgacatggatatattcggtcataacttctgctataaccaataagctttccctttgcgagccgcattaacatattccgtttttggattcggaatatctgccgcatcagaggaaagaggaaaaggacggactgaaa

SEQ ID NO: 13 - DNA, 250 bp, Zymomonas mobilis
  source 1..250: mol_type = genomic DNA
gaacgggatcaatatcgctccgatccgatccagaaaatcaaacccttccatcagaatacctcggataaagttacccaattggaaaaactagccgcccttaaaaatcaaggcatcctgacagaagaagaatttgaaaaagaaaaaagacgtattttggccttataaaagacgaaaatatttcttacattgcccccatctcaaagacagtccgcctttcaaaatagatatcacaaaatcgggaaacagaatt

SEQ ID NO: 14 - DNA, 2634 bp, synthetic construct
  source 1..2634: mol_type = other DNA
atgagattcactttaatcgaagcggttgctcttactgccgtctctttagccagcgctgatgaattggcctattccccgccgtattatccctcaccttgggccaatggccagggtgactgggcagaagcatatcagcgcgctgttgatatagtctctcagatgacattggctgaaaaggtgaatttgacaacggggactggatgggaattggaattatgtgttggtcagacagggggtgttccccgattggggattccgggaatgtgtgcacaggatagccctttgggtgttcgtgattccgattataattctgcgtttccagccggtgtcaatgtggccgcaacctgggacaaaaatctggcttatctgcgcgggcaagcaatgggtcaggagtttagtgataaaggtgctgatatccaattgggtccagctgccggccctctgggtcggagtcccgatggcggtcgtaattgggaaggcttttccccggatccggccttatccggtgtgctgtttgcagaaacaattaagggtattcaagatgctggtgttgttgcaacggctaaacattatattgcctatgagcaggaacattttcgtcaggcgcctgaagctcaaggctatggattcaatattaccgagagtggaagcgcgaacttagacgataaaacgatgcatgagttatatttgtggccattcgcggatgccattcgtgcaggtgccggtgctgtgatgtgctcgtataatcagattaataactcatatggctgtcagaatagctacacactgaataagctgcttaaggcagaattaggtttccagggctttgtcatgagtgattgggcggctcatcatgccggtgtgtcaggtgctttggcagggttggacatgtctatgccgggggacgtcgattatgactcaggcacgtcttattggggtaccaatttgaccatctcagttctgaacgggacggttccgcaatggcgagttgatgatatggctgtacgaatcatggccgcctattacaaagtcggccgtgaccggctgtggacaccgccaaactttagctcatggaccagagatgaatatggcttcaaatattattatgtctcggaaggaccgtatgaaaaggtcaatcagtttgttaatgtgcaacgcaatcatagcgaattgatccgccgtattggagcagacagcacggttctgttgaaaaacgatggggctcttccgttgactggaaaagaacgcttggtcgcccttatcggggaagatgcgggttccaatccttatggtgccaacggctgcagtgatcgtgggtgtgataatggaacattggcgatgggctggggaagtggcacagccaattttccctacttggtgacccccgaacaggccatctccaatgaagttcttaagaataaaaatggcgtattcaccgcaaccgataattgggctattgatcaaattgaggcgttagctaaaaccgccagtgtatctcttgtctttgtcaatgccgactctggtgaaggttatattaatgttgatgggaacctgggtgatcgccgtaacctgaccttatggagaaatggcgataatgtcatcaaggctgctgctagcaactgcaacaatacgatcgttattattcactctgtcggcccagtcttagttaatgaatggtatgacaatcctaatgttaccgctattttatggggtggtttacctggtcaggaatctggcaattcccttgccgacgtgttatatggccgtgtcaacccaggtgccaaatcgcccttcacctggggcaaaactcgtgaagcctaccaagattatttgtataccgagcccaataacggcaacggagcgccacaggaagattttgtcgaaggcgtctttattgattatcgcggatttgataaacggaatgaaacacctatctatgaatttggctatggtctgagctataccaccttcaattattcgaaccttcaggtggaagttttaagcgcccctgcgtacgaacctgcttctggcgaaactgaggcagcgccgacttttggagaagtcggaaatgcgtcggattatctttaccccgatggtctgcaaagaatcaccaaattcatatatccctggttgaactcaaccgatcttgaggcatcttctggggatgctagctatgggcaggatgcctcagactatcttccagagggggccaccgatggctctgcgcaaccgattctgcctgccggtggtggtgctggcggcaatcctcgcctgtatgacgaacttatccgcgttacggttactattaaaaataccggcaaagttgcaggtgatgaagttcctcaactggtatccagacaacaaattcgaaccaaatcggatcaagctaatgaatcgcagtatgtttctcttggcggccctaatgaacccaaaatcgtgctgcgtcaattcgagcggatcacgctgcaaccgtcggaagaaacgcaatggagcacgactctgacgcgccgggatcttgcgaattggaatgttgaaacgcaggattgggaaattacgtcctatccgaagatggtgtttgtcggaagctcctcgcggaaattaccgcttcgggcgtctctgcctacagttcattaa

SEQ ID NO: 15 - DNA, 2580 bp, Aspergillus welwitschiae
  source 1..2580: mol_type = genomic DNA
atgaggttcactttgatcgaggcggtggctctgactgccgtctcgctggccagcgctgatgaattggcctactcccctccgtattacccctccccttgggccaatggccagggtgactgggcggaagcataccagcgcgctgttgatatcgtctcgcagatgacattggctgagaaggtcaatttgactacgggaactggatgggaattggaattatgtgttggtcagactggaggtgttccccgattgggaattccgggaatgtgtgcacaggatagccctctgggtgttcgtgactccgactacaactctgcgttccccgccggtgtcaacgtggccgcaacctgggacaagaatctggcttaccttcgtggccaggctatgggtcaggagtttagtgacaagggtgctgatatccaattgggtccagctgccggccctctcggtagaagtcccgacggcggtcgtaactgggagggcttctcccccgacccggccctcagtggtgtgctctttgcagagacaatcaagggtattcaggatgctggtgtggttgcaacggctaagcactacatcgcctacgagcaggagcatttccgtcaggcgcctgaagctcaaggctacggattcaatattaccgagagtggaagcgcgaacctcgacgataagactatgcatgagctgtacctctggcccttcgcggatgccatccgtgcaggtgccggtgctgtgatgtgctcgtacaaccagatcaacaacagctatggctgccagaacagctacactctgaacaagctgctcaaggctgagctgggtttccagggctttgtcatgagtgattgggcggctcaccatgccggtgtgagtggtgctttggcgggattggacatgtctatgccgggagacgtcgattacgacagtggcacgtcttactggggtaccaacttgaccatcagtgtgctcaacgggacggtgccccaatggcgtgttgatgacatggctgtccgcatcatggccgcctactacaaggtcggccgtgaccgtctgtggactcctcccaacttcagctcatggaccagagatgaatacggcttcaagtactactatgtctcggagggaccgtatgagaaggtcaaccagttcgtgaatgtgcaacgcaaccatagcgagttgatccgccgtattggagcagacagcacggtgctcctcaagaacgatggcgctcttcccttgactggaaaggagcgcttggtcgcccttatcggagaagatgcgggttccaatccttatggtgccaacggctgcagtgaccgtgggtgcgacaatggaacattggcgatgggctggggaagtggcactgccaactttccctacttggtgacccccgagcaggccatctcgaacgaggtgctcaagaacaagaatggcgtattcactgcgaccgataactgggctattgatcagattgaggcgcttgctaagaccgccagtgtctctcttgtctttgtcaacgccgactctggtgagggttatatcaatgtcgacggaaacctgggtgaccgcaggaacctgaccctgtggaggaacggcgacaatgtgatcaaggctgctgctagcaactgcaacaacacgatcgttattattcactctgtcggcccagtcttggttaacgagtggtacgacaaccccaatgttaccgctattctctggggtggtcttcccggtcaggagtctggcaactccctcgccgacgtgctctacggccgtgtcaaccccggtgccaagtcgcccttcacctggggcaagactcgtgaggcctaccaagattacttgtacaccgagcccaacaacggcaacggagcgccccaggaagacttcgtcgagggcgtcttcattgactaccgcggatttgacaagcgcaacgagactcctatctatgagttcggctatggtctgagctacaccaccttcaactactcgaaccttcaggtggaggttctgagcgcccctgcgtacgagcctgcttcgggcgagactgaggcagcgccgactttcggagaggtcggaaatgcgtcggattacctctaccccgatggactgcagagaatcaccaagttcatctacccctggctcaacagtaccgatcttgaggcgtcttctggggatgctagctatgggcaggatgcctcagactatcttcccgagggagccaccgatggctctgcgcaaccgatcctgcctgccggtggtggtgctggcggcaaccctcgcctgtacgacgagctcatccgcgtgtcggtgactatcaagaacaccggcaaggttgcgggtgatgaagttcctcaactgtatgtttctcttggcggccctaacgaacccaagatcgtgctgcgtcaattcgagcgtatcacgctgcagccgtcggaagagacgcagtggagcacgactctgacgcgccgtgaccttgcgaactggaatgttgagacgcaggactgggagattacgtcttatcccaagatggtgtttgtcggaagctcctcgcggaagctgccgctccgggcgtctctgcctactgttcac

SEQ ID NO: 16 - DNA, 2583 bp, Aspergillus luchuensis
  source 1..2583: mol_type = genomic DNA
atgaggttcactttgattgaggcggtggctctcactgctgtctcgctggccagcgctgatgaattggcttactccccaccgtattacccatccccttgggccaatggccagggcgactgggcgcaggcataccagcgcgctgttgatattgtctcgcagatgacattggctgagaaggtcaatctgaccacaggaactggatgggaattggagctatgtgttggtcagactggcggggttccccgattgggagttccgggaatgtgtttacaggatagccctctgggcgttcgcgactccgactacaactctgctttcccttccggtatgaacgtggctgcaacctgggacaagaatctggcatacctccgcggcaaggctatgggtcaggaatttagtgacaagggtgccgatatccaattgggtccagctgccggccctctcggtagaagtcccgacggtggtcgtaactgggagggcttctcccccgacccggccctaagtggtgtgctctttgcagagaccatcaagggtatccaagatgctggtgtggtcgcgacggctaagcactacattgcctacgagcaagagcatttccgtcaggcgcctgaagcccaaggttatggatttaacatttccgagagtggaagcgcgaacctcgacgataagactatgcacgagctgtacctctggcccttcgcggatgccatccgtgcgggtgctggcgctgtgatgtgctcctacaaccagatcaacaacagctatggctgccagaacagctacactctgaacaagctgctcaaggccgagctgggtttccagggctttgtcatgagtgattgggcggctcaccatgctggtgtgagtggtgctttggcaggattggatatgtctatgccaggagacgtcgactacgacagtggtacgtcttactggggtacaaacctgaccgttagcgtgctcaacggaacggtgccccaatggcgtgttgatgacatggctgtccgcatcatggccgcctactacaaggtcggccgtgaccgtctgtggactcctcccaacttcagctcatggaccagagatgaatacggctacaagtactactatgtgtcggagggaccgtacgagaaggtcaaccactacgtgaacgtgcaacgcaaccacagcgaactgatccgccgcattggagcggacagcacggtgctcctcaagaacgacggcgctctgcctttgactggtaaggagcgcctggtcgcgcttatcggagaagatgcgggctccaacccttatggtgccaacggctgcagtgaccgtggatgcgacaatggaacattggcgatgggctggggaagtggtactgccaacttcccatacctggtgacccccgagcaggccatctcaaacgaggtgctcaagaacaagaatggtgtattcaccgccaccgataactgggctatcgatcagattgaggcgcttgctaagaccgccagtgtctctcttgtctttgtcaacgccgactctggcgagggttacatcaatgtcgacggaaacctgggtgaccgcaggaacctgaccctgtggaggaacggcgataatgtgatcaaggctgctgctagcaactgcaacaacaccattgttatcattcactctgtcggcccagtcttggttaacgaatggtacgacaaccccaatgttaccgctattctctggggtggtctgcccggtcaggagtctggcaactctcttgccgacgtcctctatggccgtgtcaaccccggtgccaagtcgccctttacctggggcaagactcgtgaggcctaccaagattacttggtcaccgagcccaacaacggcaatggagccccccaggaagacttcgtcgagggcgtcttcattgactaccgcggattcgacaagcgcaacgagaccccgatctacgagttcggctatggtctgagctacaccactttcaactactcgaaccttgaggtgcaggttctgagcgcccccgcgtacgagcctgcttcgggtgagactgaggcagcgccaacttttggagaggttggaaatgcgtcgaattacctctaccccgacggactgcagaaaatcaccaagttcatctacccctggctcaacagtaccgatctcgaggcatcttctggggatgctagctacggacaggactcctcggactatcttcccgagggagccaccgatggctctgcgcaaccgatcctgcctgctggtggcggtcctggcggcaaccctcgcctgtacgacgagctcatccgcgtgtcggtgaccatcaagaacaccggcaaggttgctggtgatgaagttccccaactgtatgtttcccttggcggccccaacgagcccaagatcgtgctgcgtcaattcgagcgcatcacgctgcagccgtcagaggagacgaagtggagcacgactctgacgcgccgtgaccttgcaaactggaatgttgagaagcaggactgggagattacgtcgtatcccaagatggtgtttgtcggaagctcctcgcggaagctgccgctccgggcgtctctgcctactgttcactaa

SEQ ID NO: 17 - DNA, 2583 bp, Aspergillus sclerotioniger
  source 1..2583: mol_type = genomic DNA
atgaggttcagttggatcgaggtggcggctctgacagccgcctcggtggtcagcgctgatgaattggcgtactctccccctttctacccttctccctgggccaatggcaagggtgactggacagacgcgtacaagcgtgcggtcgacattgtttcgcagatgacactggccgaaaaggtcaatctgacaacgggaactggatgggaattggaaaagtgtgtcggtcagaccggcggtgttcctcgattgggaataccgggcatgtgtggtcaggatagtcctctgggtgttcgtgactctgactacaactcggctttccctgctggcatcaacattgccgcttcctgggacaaggacctcgcgtacctccgtggcaaggccatgggccaggagttcagtgacaagggtgctgatattcaattgggccccgttgctggtcccctcggtagaagtcccgacggcggtcgtaactgggaggggtttgctccagacccggccctgactggtgtgctctttgcagagaccatcaagggtatccaggatgctggcgtggttgcgacggctaagcacttcatcggctatgagcaagagcatttccgccaggcacctgaggctcaaggatatgggtacaacatcaccgagagtggcagcgcgaacatcgacgataagaccatgcacgagctgtacctctggccctttgcggatgctatccgcgcaggtgttggcgcggtcatgtgctcctacaaccagatcaacaacagctacggttgccagaacagctatgctctgaacaagctccttaagtctgaactggaattccagggcttcgtgatgagcgactgggaggcccaccacagtggagtcggtgctgctttggccggtctggacatgtctatgcccggagatgtcacctacgacagtggcacgtcttactggggtgccaacctgaccatcagtgtcctgaacggcacggttccccagtggcgtgttgatgatatggctgtccgtatcatggccgcctactacaaggtcggtcgggatcgtctgtggacgcctcccaacttcagttcatggacccgcgatgaatacggctacaagtactactattcctccgagggaccgtatgagaaggtcaaccagtttgtcaacgtgcagcgcaaccatagcgggttgatccgccgtattggagctgacagcacggttcttttgaagaacgagaacgttctgcccctgactggcaaggagcgcctggtcggtcttatcggagaagacgcgggctccaacgcttacggcgccaacggctgcagtgaccgtggatgcgacaacggtactctggctatgggctggggcagtggtacctcgaactacccttacttgatcactcccgagcaggcgatccaggctgaggtgatcaagaacaagggcaatgtatttgccgtgaccgacaactgggccattgaccagattgaggcactcgccaagcaatccagtgtctctcttgtctttgtcaacgccgactctggtgaaggttacatcgatgtcgatggcaacatgggtgaccgcaacaacattacgctctggaggaacggtgacaacgtggtcaaggccgctgccaacaactgcaacaacaccattgtcatcatccactccgtcggcccggtcctcgtcaccgagtggtacgaccaccccaatgtcaccggcatactgtgggctggcttgcccggccaggagtctggcaactcgctcgccgatgtgctctacggccgtgtcaacccgggtgccaagtcgcccttcacctggggtaagaccagggagtcctaccgcgactacctgatcaccgagcccaacaatggcgatggagccccacaggaagacttcaccgagggcatcttcatcgactaccgcgggttcgacaagcgcaatgagaccccaatctacgagttcggctatggcctgagctacaccaccttcaactactcgaatttgaacgtgcaggtcctgaatgcctcgtcgtacactccttcttctggcgagactgaggctgccccgacttttggagagatcggcaacgcgtcggactacctctaccctagcggactgaagaagattaccgacttcatctacccctggcttaacagcacggacctcctggaagcttccggcgacgccagctacggtctgaacgcgtcggagtatatccccgagggaggcaccgatggctctgcccagccgatcctggctgctggtggtggccctggcggtaaccccggtctgtatgacgagctgattcgcgtttcggcgaccatcaagaacaccggcaagcttgcgggtgacgaggttcctcaactgtacgtttcgcttggaggtcccgatgacgccaaaattgtgttgcgcaagttcgaccgcatctctctcaagccatcgcaggaggttgagtggagcacgactctgacacgacgcgacctggcaaactgggatgttgcggcgcaggactggaccatcacgtcttatcccaagacggtgtatgtcggtagctcttcgcggaagctgccgctccgcgcgtcactgcctactgtccaatag

SEQ ID NO: 18 - DNA, 2586 bp, Aspergillus tanneri
  source 1..2586: mol_type = genomic DNA
atgaggtttggttggtttgaggcggcggtcgtgaccgctgcctcagtggtcagtgcccaggatgatcttgctttctctcctccgtactatccttccccgtgggccaatggccagggagaatgggccgacgcgtatgaacgtgccgtcgacatcgtctcccagatgacgttggcggagaaggttaacctcactactggaacagggtggatgttggataaatgtgtgggtcagacaggaagtgttcccagactcggactattaagtctctgcttgcaagacagtcccttgggtattcggttttcggattacaattcggcattccccgcaggtgttaatgtcgccgcaacatgggacaagcaactggcgtacctccgcggtagggcaatgggtgaggaattcagcgacaagggaatcgacgttcaattaggccctgctgctgggcctctcggcagacaccccgatggtggtcggaactgggaaggtttctcccctgaccctgcccttaccggtgtacttttcgcagaaaccataaaaggcatccaggatgctggtgtgatcgcgacggccaagcattacattttgaatgaacaggagcatttccgtcaagttcccgaagcccttgcggctggattcaatatttcggactccctgagctccaatctagacgacaaaaccatgcatgagctgtacctctggcccttcgcagatgcagtgcgcgcgggtgtgggtgctgtgatgtgctcctacactcagatcaataacagttacggctgccagaatagcgaaactctgaataaacttctcaaggcggagcttggcttccagggttttgtcatgtcggactggagtgcgcaccacagcggtgtcggctctgccttggcggggctggatatgtcaatgcccggggatatcgccttcaatgacggcaattcgtacttcggggcaaacttgacgattggcgtcctcaacggaaccatccctcagtggagagtggacgacatggctgtccgcatcatggctgcttactataaggttggccgggatcgccaccacgcgcctcccaacttcagctcatggaccagggacgaatacggctacgagcacgctgcagtttcggagggcgcgtatgaaagggtgaaccaatttgtcgacgtgcagcgggaccatgcggaaatcatccgtcgtgtgggtgccgagagcattgttctcttgaagaacgagaacgctctgccgttgagtgggaaggagaagaaggtcgttatcctcggagaagatgcgggatccaacccttggggtgccaatggctgcgaaaaccgtggatgtgacaatgggacccttgccatggcttggggcagtggcactacagagttcccctacctcgtcactcccgaacaggcgatccagaacgaggtcctccgaggtcgtggtaatgtatttagtgtcaccgacaacggggctctgagtcagatggcggcgcttgcctctcaatctagtgtcgctcttgtctttgtcaatgccgactcgggtgagggattcatcaccgtggatggaaacgagggagatcgcaagaacctcactctctggaagaacggagagaacgtgatcaaaaccgctgcccaaaactgcaacaacaccgtcgtgatcatccactccgtcggagccgttctggttgacgaatggtatgaccaccccaatattaccggcattctttgggcgggtctgcctggtcaggagtccgggaactccctcgccgacgtgctgtacggccgtgtcaaccctggcggtaagacccccttcacctggggtcgaacccgcgaatcgtatggggcacccctggtcaccgatgcgaacaacggccacggtgcgccccagtcggacttcgaagaaggggtgtttattgactaccgtcatttcgacaagttcaacgagactcccatctacgagtttggctacggccttagttacaccactttcggctactcgggactgcaaattgagcccctgaacgcgcccaagtacgtccccaactccgggaagaccgaagcggcgccaactttcggcgatgttgcagaggtctcagactatgtgtatccaaagggactgcggagaatccgtgagttcatctacccctggctgaattccaccaatctgaagaagtcctccggcgatgccacttatggaggggaggactcgacgtacatccccgatgacgccaccgatggttctcctcaggaccttctgcctgccagcggtggtcccggaggcaatccaggtctctaccaggatctggtgcgggtgtcggttacgatcacgaacacgggcaacgtggccggctacgacgtgcctcagctttatgtttcccttggtggccccaacgagccgaaggtggtactccgcaaattcgatcgatttaagctggatccgtcgcagcaggtggtgtggtcgaccacgctgaatcgtcgcgacctgtccaactgggacgtgacggggcaggactgggtcatcaccgagtatcccaagaaggtcttcgttggtagctcgtcccggaaattgccgctacatgcttccttgccggagatggagtaa

SEQ ID NO: 19 - DNA, 2586 bp, Aspergillus glaucus
  source 1..2586: mol_type = genomic DNA
atgaagctcggctggttggagtttgttgccgtcacggcctcggtggcccaggctaaggacctcgcgtactctcccccctactatccctcgccatgggccgatggtcaacccgccgaatggtccaacgcgtacaaacgcgccgtcgacatcgtctccaacatgactttggcggaaaaggtcaacttgaccactggtactggctggcaattggaagaatgtgttggtcagactggtagtgttccacgacttggtatctggggtatctgtttgcaggactcgcctcttggtatccgttatggcgatcacagctctggcttccccgctggtctcaacgtcgccgcgacctgggaccgcaagctcgcctacctccgtggtgaggctatgggtcaggaattcagcgacaaaggaattgacgtccaattgggtcccgttgctggccctatcggcagatctcccgacggtggtcgcaactgggagggtttcgcccccgacccggttctgaccggtgtgctcatggccgagactatcaagggtattcaggatgccggtgtcatcgctactgctaagcactacatcggcaacgagcaggagcatttccgtcaggtttccgaggctctcgactatggttacaacatcactgagactgccagctccaacatcgacgacaagaccatgcacgagctctacttgtggcctttcgccgatgctgttcgtgctggtgtcggttccgtcatgtgctcgtacaaccagatcaacaacagctatggttgccagaacagtcatttgctgaacaagctgctcaagcacgagctgggcttccagggcttcgttatgaccgactggggtgctcaccatagcggtgtcgcctccacccttgctggtactgacatgtcgatgcccggtgatatctcgtttgacgatggtatgtcgtacttcggtcccaacctgactgttgctgttctcaacggtactgttcccgagtggcgtgtcgacgacatggccgttcgtatcatgtctgctttctacaaggttggccgtgaccgccagcgcacgcctcccaacttcagctcctggaccaacgacgagtacagctacgcccactccgccgtccaggaaggctggaagcaggtcaaccagcacatcaatgtccagcgcaaccactccgagatcatccgtgaggttggcgaagccagcactgttctcttgaagaacaagggtgctcttcctctgactggcgacgagggctccgttggtattctaggcgaagatgccgggtccaacgcgtatggtgccaacggctgcgaggaccgtggatgcgacaatggtacccttgcgatggcctggggtagcggttccgccgaattcccttacctggttactcccgagcaggctatccagaacgagattctcaaccgggacgtcaagcaccccgtcttcgccgtcactgacaactgggctttggatcagatggcctccattgcctctcaatctgatgtttctcttgtcttcgtcaacgccgatgctggtgagggtttcctcgtcgtggatggtaatgagggtgaccgcaataacatcaccctctggaagaacggtgagaatgtcatcaagactgtcagcgagaactgcaacaacaccattgtgatcatgcacaccgtcggacccgtcctcatcgaccagtggtacgacaaccccaacatcaccgccattgtctgggctggtctgcctggccaggaatccggaaacgccattgccaacgttctctacggccgtgtcaaccctggtggcaagagccccttcacctggggtaagagccgcgaggcatacggtgccccgctcctcaccgagacaaacaacggcatcggcgctcctcaggtcgactttactgagggtcagttcatcgactaccggcgcttcgacaagtacaacgagacccctatctacgagtttggctacggtctgagttataccaccttcaagtactccaacctccacgtccaggccctgaacgcctccaagtacgttcccaccactggcaagaccagtgccgcgcccaagcttggtgaggttggcaaggcttcgaactacgtcttccccaatggattcgagcgcaccaccaagttcatctacccctggttgaactcgaccgatctgaagaagtccgccaacgaccccgagtacggcctcgagatctcaaagtacatccccgagaacgctcaggacgaatcctcccaggcccgtttgccagccagcggcggccaaggaggcaacaccggtctctacgacgaactcttccgcgtctccgccacgatcaagaacaccggcaaggtcgcaggtgacgaagttccccagctgtacgtctctctcggcggccccaacgagcccaaggttgtcctgcgcaactttgaccgcatcgccctccagcctggtcaggaggtcgtgtggtcgaagactcttacccgtcgtgacctttccaactgggacgttgccgctcaggactgggctatcacgcagcataccaagaaggtgtttgttgggagctcgtcgcgcaagctgcctttgcgggcttcgttacctcgcgtgcagtag

SEQ ID NO: 20 - DNA, 2589 bp, Penicillium argentinense
  source 1..2589: mol_type = genomic DNA
atgaagctcgggttgctcgaagccgccgtcttgacggccgcctcggccgccactgcgtcggacctggcatactcgcccccgtactatccgtccccgtggatgaccggcgagggcgactgggccgaggcctaccgccgcgccgtcgacttcgtctccaacctcacactggctgaaaaggtcaacttgaccactggtgcgggctgggagcaagaacgctgcgtcggggagacgggtggtattccccgactgggaatgtggggcatgtgcttgcaagactcgcccctgggcatccgcgacagtgactacaactccggtttcgccgcgggcgtcaatgttgccgccacatgggacaagagactcgcctaccagcgtggtctggcgatgggcgaggagcaccgcgacaagggtgtcgatgtgcagctcggtcctgtggccggcccactgggacgaagccccgatggcggccgtggctgggaagggttctcgcctgatcctgtcctcaccggtgttatgatggcggagactatcaagggtattcaggatgctggtgtcattgcttgcgccaagcactatatcggaaacgagcaggaacacttccgccagtcgggcgaggctcagggctatgggtacaacatcactgagagtgtgagctccaacatcgacgacaagactatgcacgaactgtacctctggccctttgtcgactctatccgcgccggtgtcggctctgtcatgtgctcgtacaatcagatcaacaacagctatggatgccagaacagcgagaccttgaacaggttgctcaaggctgagctgggtttccagggcttcgtcatgtcggattggggtgcgcaccatagcggtgtcagcgctacccttgctggtctggatatgtccatgcccggtgatgtcatcctcggtagcccgtactccttctggggcacgaacctgaccatctccgtgctgaacggtaccgtccccgaatggcgtatcgacgatatggccgtccgcatcatgtcggcctactacaaggtcggccgtgaccgtgtccgtgtccctcccaacttcagctcctggactcgtgatgagtatggctttgagcacttcatggtcagcgagaactacatcaagctcaacgagcgcgtcaatgtccagcgcgaccacgccgcgggtatccgcaagcttggctctgacagcaccgttctgctgaagaacaagggtgctctgcccttgactcacaatgagaagttcgttggtattctgggtgaggatgctggttccaaccctgctggcgctaatggctgtgcggaccgtggttgcgatgacggcacccttgctatgggctggggtagtggtactgctaacttcccttacctgattacccccgagcaagccattcagaacgaggttctgaactatggcaatggccagacaaatgtgtttgccgtgaccaacaacaccaacacggagcagattgctgccattgctgcgcagtcgagcgtcgctctcgtcttcgtaaacgccgactccggcgaaggcttcatcaatgtcgacggcaacgagggtgaccgcaagaacctaaccctctggaagaacggcgaacacctcatcaaaacagctgcggcaaactgcaacaacaccgtcgtcatcatgcacacccccggcgccgtcctaatcagcgactggtacgagcacgacaacatcaccgccatcctctgggccggtctaccgggccaagaaagcggccgcagtatcacagacattctctacgggcgcgtgaacccaggcggcaaaacacccttcacctggggaaagactcgcaaagactacggcgcaccgctcctcacacaacccaacaacggccatggtgcgccgcagcaagacttcacagagggagtcttcatcgactaccgccgcttcgacaaggagcaggaagagcctgtctacgaattcgggtacggactcagctacacgaatttcgaattcagcgatctgcacatcaagtcccttaacgcagacaagtatgtcccgacaacggggacaaccaagcctgcccccgtcttcggcaaaatcggccaggcatccgactatctcttcccgaagggcatccaccgtgtcacccaatacctctacccctacctgaacagcacaaacctcaagtcctcctccggcgacccctactacggcgctaagacagaggagtacatccccgccggcgcaaccaacggctccgcgcaaacccgcctcccatccagcggtgccaacggcggtaatgcgggtctcttcgaggatctgtaccaggttaccgtgacaattaccaacaccggctctgtgcagggcgacgaagttccccagttgtacgttagtctgggcggggagaataaccccgttaaggtcctgcgtgcgtttgacaggatcaccattgcgcctggccagaaggcacagtggactactactcttacccgccgcgatttgtcgagttgggatgttgcaaaacagaattgggtagttactcctgcgcagaagaaggtgtatgttggaaattcgtcgcggagacttccgcttgagtcggagttgcccgcttcgaaatga

SEQ ID NO: 21 - DNA, 2583 bp, Aspergillus wentii
  source 1..2583: mol_type = genomic DNA
atgaggttcggttggttggaggtagccgctcttacggctgcctccgtggtcagtgccaaggatgacctggcattctcccctcccttctacccctctccgtgggcaaacggtgagggtgaatgggcagactcgtacaagcgcgctgtcgagtttgtttcgaacttgaccttggctgagaaggtcaaccttacaactggttctggctggcagcaagagagatgtgttggtgagacgggcgaagtttctagacttggattctggggtatttgtttgcaggattctcccctgggtattcgttttggcgaccactcctccggcttcccctccggcctcaacgttgctgcaacctgggacaaaaagcttgcctacctccgtggtaaggcaatgggcgaggaattccgtgacaagggcattgatgttcaactgggacctgtcgctggtccccttggcgctttccctgatggtggtcgaaactgggagggtttcgctcccgaccctgtgctgactggtttcctgatgtcggagactatcaagggtatccaagatgctggtgttattgctactgctaagcattacatcggtaatgagcaggagcatttccgccaaagcggcgaggctaagggttatggttacaacattactgaaagttcgagctcaaacattgatgacaagacaatgcacgagttgtatctctggccttttgctgatgccgttcgtgctggtgtcggcgcgttcatgtgctcatacaaccagatcaacaacagctatggctgctccaacagctacctgatgaacaaactcctcaagtctgaactaggcttccaaggatttgtcatgagtgattggggtgcacaccacagcggtgttggcgctgctttggctggtttggatatgtccatgcctggtgacactgtcatgggcgacccctacaccttctggggaaccaacctgaccatctcggttctcaacggcaccgtccccgagtggcgtgtggatgacatggccgtccgtatcatggctgcttactacaaggtgggccgcgatgaggttcgcacccctcccaacttcagctcgtggaccaccgaagaattcggatacgcgcattatgctgctcaggaaggatacgagaagaccaactggaatgtcaatgtccgccgcaaccacgccaaggtcatccgcgaaatcggttcggccagcacggttcttctgaagaacaatggcgtgctccccctgaccggtgatgaggactatgtcggaattctgggcgaggacgccggagctaacccctatggtgccaacggctgtgaagaccgtggctgcgacaatggtactctcgctatggcttggggcagtggtagtgcggaattcccttaccttgtgactcccgagcaggccatccagaacgaggttctcaagggcgagggctctgtgttcgctgtgaccgataactgggctctcgagcagatggcttctattgcttcgcagtcctctatctctctcgtctttgtcaatgccgactccggagaaggtttcctcaatgtggacggaaacatgggtgaccgcaagaacttcaccctctggaaaaatggcgagaatgtgatcaagactgtcactgagaactgcaacaacaccgttgtggtcatgcacactgttggcccggtcctgatcaaggactggtatgacaaccctaacatcactgccattgtctgggctggtttgcctggccaggaaagcggaaactctctcgctgatgtgctttacggccgtgtcaaccctagtggcaagagcccattcacctggggcaagactcgcgaggcttatggtccttctctgctcaccacccagaacaacggcaatgacgctccccagcaagatttcacccagggtgtcttcattgactaccgccgatttgacaagttcaacgagactcccatctacgagttcggatacggtctgagttacaccaccttcaactactccaacctcgaagttcgatccctgaatgcatcgcggtacaccccaaccactggcaagactgatgccgcgccttctctgggcgaggctggcaaggcctcggactttttgttccccaagggactgaaccgcatcatcggctacatttatccgtggttgaactcgaccgacctgaagtcggcgtccggagagaagaactatggcatgaaggcttctgagtacattcccgagggagccaccgatggatccgcccaggagctcctgccggccggcggtggccctggtggtaaccctggtctgtatgaagacctcatcgaggtttctgctactattaccaacactggcaaggttgctggtgacgaagtcccacagctgtatgtctcccttggtggtgccgacgaccccgttcttgtcctccgtcagttcgaccgtctccacatcgaggctggaaagcaggcagtgtggaagacgactcttacccgccgtgacctcgccaactgggacgttgcggcgcaagactggaccatcacgaagagcgcgaagaaggtgtacgtgggcagctcttcgcggaacctgcccctcaaggccaagcttcccaccgttcagtag

SEQ ID NO: 22 - protein, 916 aa, Penicillium vulpinum
MSTIIAPVSKELFEYLRTHAFPEICDSFLPHAFFYQQRFLCCLCEILSSIFTAMKFEWLTVAGLTAAANANPLAYSPPYYPSPWMTGAGEWSDAYTRAVDFVSNLTLAEKVNLTTGAGWEQERCVGETGGIPRLGMWGMCMQDSPLGVRLSDYTSGFPSGINVAATWDKRLAYQRGMAMGEEHRDKGVDVQLGPVAGPLGKYPEGGRNWEGFSPDPVLTGVMMAETIKGMQDAGVIACAKHFIGNEQEHFRQSGEAQGYGYNISESVSSNIDDKTMHELYLWPFVDSIRAGVGSIMCSYNQINNSYGCANSYALNKLLKGELGFQGFVMSDWGAHHSGVSSTLAGLDMSMPGDTYLGSPSSFWGANLTISVLNGTVPEWRIDDMAVRIMAAYYKVGRDRFRTPPNFSSWTRDEYSFEHAAVSEGWAKVNERVNVQRDHAQIIRKIGSDSTVLLKNKGGALPLTHGEKFISILGEDAGSNAYGANGCGDRGCDNGTLAMGWGSGTANFPYLITPEQAIQNEVLEYSVGKTSVFAVTDNWALTEMAALASQADVALVFVNADSGEGYINVDGNEGDRKNLTLWKNGEEIIKTASQHCNNTIVVIHSTAAVLISDWYDNDNITAIVWAGLPGQESGRSLVDVLYGRINPGGKTPFTWGKTRKDYGPPLLTVPNNGADAPQDNFEDGVFIDYRRFDKDNTEPIYEFGYGLSYTNFSFSDLKVTPLASSEYNEYKATTGKTKKAPVLGKAGKVSDNLFPEGIKPVRQYLYPWLNSTDLRASSGDPAYGMDSKDYLPEGATDGSPQDLLPSSGASGGNPDLFKNLYQVTATITNTGSVTGDEVPQLYVSLGGDDEPSKVLRQFDRVTIAPGQTLQWTTTLTRRDVSNWDVASQNWVISDAPKKVYVGNSSRKLPLSADLPPI

SEQ ID NO: 23 - protein, 1230 aa, Aspergillus terreus
MKLSILEAAALTAASVVSAQVTFTLPVNLVLDGALTVRKQDDLAYSPPYYPSPWADGHGEWSNAYKRAVDIVSQMTLTEKVNLTTGTGWELERCVGQTGSVPRLGIPSLCLQDSPLGIRMSDYNSAFPAGINVAATWDKTLAYQRGKAMGEEFSDKGIDVQLGPAAGPLGRSPDGGRNWEGFSPDPALTGVLFAETIKGIQDAGVIATAKHYILNEQEHFRQVGEAQGYGFNITETVSSNVDDKTMHELYLWPFADAVRAGVGAVMCSYNQINNSYGCQNSLTLNKLLKAELGFQGFVMSDWSAHHSGVGAALAGLDMSMPGDISFDSGTSFYGTNLTVGVLNGTIPQWRVDDMAVRIMAAYYKVGRDRLWTPPNFSSWTRDEYGFAHFFPSEGAYERVNEFVNVQRDHAQVIRRIGADSVVLLKNDGALPLTGQEKTVGILGEDAGSNPKGANGCSDRGCDKGTLAMAWGSGTANFPYLVTPEQAIQNEVLKGRGNVFAVTDNYDTQQIAAVASQSTVSLVFVNADAGEGFLNVDGNMGDRKNLTLWQNGEEVIKTVTEHCNNTVVVIHSVGPVLIDEWYAHPNVTGILWAGLPGQESGNAIADVLYGRVNPGGKTPFTWGKTRASYGDYLLTEPNNGNGAPQDNFNEGVFIDYRRFDKYNETPIYEFGHGLSYTTFELSGLQVQLINGSSYVPTTGQTSAAQTFGKVEDASSYLYPEGLKRISKFIYPWLNSTDLKASTGDPDYGEPNFEYIPEGATDGSPQPRLPASGGPGGNPGLYEDLFQVSVTVTNTGKVAGDEVPQLYVSLGGPNEPKRVLRKFERLHIAPGQQKVWTTTLNRRDLANWDVVAQDWKITPYAKTIFVGTSSRKLPLAGRLPRVHLVSNQKMKHDVIFFQTPRLLPRQRLNYSQSLRDTAHSHGTTLPSVRPSPETKKTNHQPHGTRRKTEKKNMATTARASSPPGTRPFQPPTAALLVYPATLIIGSLFSVLSPTAQGARASASDDAATAAAPVNYFARKNNIFNVYFVKIGWVWTTLAFAAILLTQPAYTAPSAQRPRRLAQAAARYALATLVWWLTTQWFFGPAIIDRGFVLTGGKCEARAEGTHAASSLPVSPLGMVSAAACKAAGGAWTGGHDVSGHVFMLVLATAMLGFEMGGVFGVEGGKGVGVWSRRFVGAVLGLSWWMLLMTAIWFHTWFEKLTGLLIALGTVYTVYILPRRVVPWRNVVGIPGV

SEQ ID NO: 24 - protein, 882 aa, Stachybotrys microspore
MALASTFLSLYIFLFAYIFTGFACADPDSLYQRQDDGLLTSPPDYPSPWADPEANGWEQAYAQARDFVSQLTLLEKVNLTTGVGWMSERCVGNTGSIPRLGLRGLCLQDGPLGMRFTDYNSAFPAGVVAGATWSRHLWHDRGRMMGEEQYGKGSDVLLGPASGPIGRAPTGGRNWEGFSVDPYHSGVAMAQVVRGIQGAGVIATAKHFVANEQEHFRQAPEAVGYGFNITESLSSNLDDKTLHELYAWPFQDAVRAGVGSIMCSYNQINNSYGCQNSKLLNGILKDEYGFQGFVMSDWQAQHAGAASAAAGLDMSMPGDTVFNTGYSFWGGNLTLGVINGTVPEWRIDDMALRIMAAFFKVGRTVGGQPDINFSSWTRETLGYIHPMAQENLERVNFQVDVRGNHANHIRESAAKGSVILKNNGVLPLSSPKFVVVIGEDAGGNPAGPNGCPDRNCDNGTLAMAWGSGTAQFPYLVTPDQALQRQALEDGTRYESILANNQWTAVQNILREPNTTTIVFADADSGEGFIDVDGNRGDRRNLTLWKDGDALIKNVSSLTSNVIVVLHTVGPVLLTEWYDNPNITAIVWAGVPGQESGNSLTDILYGRRSPGRSPFTWGRTRESYGADLLYEPNNGEGAPQQDFSEGVFIDYRHFDRETAADPENTPIYEFGYGLSWSTFEYSNLQVAKRNVRPYQPTTGNTISAPVFGAPVDPDLSQYTFPPGIRYIYRFIYPYLNTSSSGEDASGDPEYGQEADQFLPPGAIDGSPQPRHPAGGQPGGNPQLWDVLYTVTATITNTGDRMTDEVPQLYISHGGDGEPVRVLRGFDRIERIAPGESVQFRADLTRHDLSNWDVVSQNWVITDDEKTVWVGSSSRNLPLAAPLR

SEQ ID NO: 25 - protein, 857 aa, Rasamsonia emersonii
MRNGLLKVAALAAASAVNGENLAYSPPFYPSPWANGQGDWAEAYQKAVQFVSQLTLAEKVNLTTGTGWEQDRCVGQVGSIPRLGFPGLCMQDSPLGVRDTDYNSAFPAGVNVAATWDRNLAYRRGVAMGEEHRGKGVDVQLGPVAGPLGRSPDAGRNWEGFAPDPVLTGNMMASTIQGIQDAGVIACAKHFILYEQEHFRQGAQDGYDISDSISANADDKTMHELYLWPFADAVRAGVGSVMCSYNQVNNSYACSNSYTMNKLLKSELGFQGFVMTDWGGHHSGVGSALAGLDMSMPGDIAFDSGTSFWGTNLTVAVLNGSIPEWRVDDMAVRIMSAYYKVGRDRYSVPINFDSWTLDTYGPEHYAVGQGQTKINEHVDVRGNHAEIIHEIGAASAVLLKNKGGLPLTGTERFVGVFGKDAGSNPWGVNGCSDRGCDNGTLAMGWGSGTANFPYLVTPEQAIQREVLSRNGTFTGITDNGALAEMAAAASQADTCLVFANADSGEGYITVDGNEGDRKNLTLWQGADQVIHNVSANCNNTVVVLHTVGPVLIDDWYDHPNVTAILWAGLPGQESGNSLVDVLYGRVNPGKTPFTWGRARDDYGAPLIVKPNNGKGAPQQDFTEGIFIDYRRFDKYNITPIYEFGFGLSYTTFEFSQLNVQPINAPPYTPASGFTKAAQSFGQPSNASDNLYPSDIERVPLYIYPWLNSTDLKASANDPDYGLPTEKYVPPNATNGDPQPIDPAGGAPGGNPSLYEPVARVTTIITNTGKVTGDEVPQLYVSLGGPDDAPKVLRGFDRITLAPGQQYLWTTTLTRRDISNWDPVTQNWVVTNYTKTIYVGNSSRNLPLQAPLKPYPGI

SEQ ID NO: 26 - protein, 856 aa, Penicillium waksmanii
MKLGWLEAATLAAASVASASDLAYSPPHYPSPWSTGDGEWAEAYRRAVEFVSNLTLPEKVNLTTGAGWEQEKCVGETGGIPRLGMWGMCMQDSPLGIRDSDYNSGFAAGVNVAATWDKRLAYQRGLAMGEEHRDKGVDVQLGPVAGPLGRSPDGGRVWEGFSPDPVLTGVMMAQTIKGIQDAGVIACAKHFIGNEQEHFRQAGEAQGYGYNISESVSSNIDDKTMHELYLWPFVDSVRAGVGSVMCSYNQINNSYGCSNSYTLNKLLKGELGFQGFVMSDWGAHHSGVGDALAGLDMSMPGDVILGSPYSFWGTNLTISALNGTIPEWRLDDMAVRIMAAYYKVGRDRVRVPPNFSSWTRDEYGYEHFIVSENQIKLNERVNVQRDHASGIRKLGSDSTVLLKNKGALPLTHNERFVAILGEDAGSNPAGANGCSDRGCDDGTLAMGWGSGTANFPYLITPEQAIQNEYLNYGNGQTNVFAVTNNSNTEQIAAMASQATVSLVFVNADSGEGYINVDGNEGDRKNLTLWKNGEQLIKTAAENCNNTIVIMHTPSAVLVGDWYDNENITAILWAGLPVSPIFSMVASTPARRPPFTWGKTRDAYGAPLLTKPNNGQGAPQQDFSEGVFIDYRQFDKADEEPIFEFGFGLSYTKFEFSDVHVTPLKADKYTKTTGRTKPAPVLGKIGEASDYLFPSGIKRVTQYLYPWLNSTNLKESSGDPYYGEKAEKYIPAHARDGSAQQLLPASGPSGGNAGLFEDLFQVTATVTNTGSVVGDEVAQLYVSLGGEGDPVKVLRAFDRITIAPGQNAQWTTTLTRRDLSNWDVASQNWVISDAPKKVYIGNSSRHLPVSIELPSTK

SEQ ID NO: 27 - protein, 801 aa, Aspergillus udagawae
MTLAEKVNLTTGTGIFMGPCAGQTGSALRFGIPNLCLHDSPLGVRNSDHNTAFPAGITVGATFDKDLMYDRGAEMGEEFRGKGINVLLGPSVGPIGRKPRGGRNWEGFGADPSLQAIGGAQTIKGIQSKGVIATIKHYIGNEQEMYRMSNIGQRGYSSNIDDRTLHELYLWPFAEGVRAGVGAVMAAYNDVNSSACSQNSKLLNEILKDELGFQGFVMTDWLGQYGGVSSALAGLDMAMPGDGAVPLLGDAYWGSELSRSILNGSVPVSRLNDMVTRIVATWYKMGQDGDYPLPNFSSNTQDATGPLYPGALFSPSGVVNQYVNVQADHNITARAIARDAITLLKNDDNILPLRKNDSLKIFGADAGPNPDGLNSCADQGCNKGVLTMGWGSGTSRLPYLVTPQQAIANISSNATFYITDSFPSNLAVGSGDIALVFINADSGENYITVEGNPGDRTSAGLNAWHNGDKLVKDAAAKFSKVVVVIHTVGPILMEEWIDLPSVKAVLVAHLPGQEAGWSLTDILFGDSSPSGHLPYTIPRSESDYPSSVGLLSQPLVQIQDTYTEGLYIDYRHFLKANITPRYPFGHGLSYTTFSFSQPTLSVRTAVGSAYPPTRAPKGPTPSYPTTIPNPSEVAWPKNFDRIWRYLYPYLDDPAGAAKNSSKTYPYPTGYTTVPKPAPRAGGAEGGNPALFDVAFAVSVTVTNTGKRPGRAVAQLYVELPNTLGVETPSRQLRQFAKTKVLAPGARETLTMEITRKDISVWDVVVQDWKAPVQGQGVKFWLGESVLDMRAVCEVGGACTII

SEQ ID NO: 28 - protein, 753 aa, Aspergillus transmontanensis
MLLTVDAQRTIIRLSLRGQAMGEEFSDKGIDVQLGPAAGPLGAHPDGGRNWEGFSPDPALTGVLFAETIKGIQDAGVIATAKHYIMNEQEHFRQQPEAAGYGFNVSDSLSSNVDDKTIHELYLWPFADAVRAGVGAVMCSYNQINNSYGCENSETLNKLLKAELGFQGFVMSDWTAHHSGVGAALAGMDMSMPGDVTFDSGTSFWGANLTVGVLNGTIPQWRVDDMAVRIMAAYYKVGRDTKYTPPNFSSWTRDEYGFAHNHVSEGAYERVNEFVDVQRDHADLIRRIGAQSTVLLKNKGALPLSRKEKLVALLGEDAGSNSWGANGCDDRGCDNGTLAMAWGSGTANFPYLVTPEQAIQNEVLQGRGNVFAVTDSWALDKIAAAARQASVSLVFVNSDSGEGYLSVDGNEGDRNNITLWKNGDNVVKTAANNCNNTVVIIHSVGPVLINEWYDHPNVTGILWAGLPGQESGNSIADVLYGRVNPGAKSPFTWGKTRESYGSPLVKDANNGNGAPQSDFTQGVFIDYRHFDKFNETPIYEFGYGLSYTTFELSDLHVQPLNASQYTPTSGMTEAAKNFGEIGDASEYVYPEGLERIHEFIYPWINSTDLKASSDDSNYGWEDSEYIPEGATDGSAQPRLPASGGAGGNPGLYEDLFRVSVKVKNTGNVAGDEVPQLYVSLGGPNEPKVVLRKFERIHLAPSQEVVWTTTLTRRDLANWDVSAQDWAVTPYPKTIYVGNSSRKLPLQVSLPKAQ

SEQ ID NO: 29 - protein, 694 aa, Penicillium oxalicum
MKLEWLEATVLAAATVASAKDLAYSPPFYPSPWATGEGEWAEAYKKAVDFVSGLTLAEKVNITTGAGWEQERCVGSVMCSYNQINNSYGCSNSYTLNKLLKGELGFQGFVMSDWGAHHSGVGDALAGLDMSMPGDVILGSPYSFWGTNLTVSVLNSTIPEWRLDDMAVRIMAAYYKVGRDRHRTPPNFSSWTRDEYGYEHFIVQENYVKLNERVNVQRDHANVIRKIGSDSIVMLKNNGGLPLTHQERLVAILGEDAGSNAYGANGCSDRGCDNGTLAMGWGSGTANFPYLITPEQAIQNEVLNYGNGDTNVFAVTDNGALSQMAALASTASVALVFVNADSGEGYISVDGNEGDRKNMTLWKNGEELIKTATANCNNTIVIMHTPNAVLVDSWYDNENITAILWAGMPGQESGRSLVDVLYGRTNPGGKTPFTWGKERKDWGSPLLTKPNNGHGAPQDDFTDVLIDYRRFDKDNVEPIFEFGFGLSYTKFEFSDIQVKALNHGEYNATVGKTKPAPSLGKPGNASDHLFPSNINRVRQYLYPYLNSTDLKASANDPDYGMNASAYIPPHATDSDPQDLLPASGPSGGNPGLFEDLIEVTATVTNTGSVTGDEVPQLYVSLGGADDPVKVLRAFDRVTIAPGQKLRWTATLNRRDLSNWDVPSQNWIISDAPKKVWVGNSSRKLPLSADLPKVQ

SEQ ID NO: 30 - protein, 707 aa, Histoplasma mississippiense
MLLPIAFASLAVATEHLTESPPYYPSPWASGQGGWEDAVERARDFVSQLTLVEKVNLTTGVGWMQENCVGQVGSIPRMGLHSLCMQDGPLGIRFADYVSAFPAGVNVGATFSKELAYLRGKAMGEEHRDKGVDVVLGPVVGPLGRSPDGGRNWEGFSPDPVNSGLLVAETIKGIQSASVIACVKHFIGNEQERFRQGPEAQGYGFDISESSSSNIDDVTMHELYLWPFADAVRAGVGSVMCSYNQINNSYGCGNSYTQNKLLKAELGFQGFIMSDWQAHHSGVGSALAGLDMSMPGDTVFGTGRSYWGPNLTIAVANGTIPEWRVDDMAVRIMAAYFKVGREAAKVPVNFNSWTRDEYDYTHALVKEGYGKVNERINVRAKHASIIRQVGAASVVLLKHTGSLPLTGLEKNAAVIGEDAGPNLWGPNGCPDRRCDNGTLAMGWGSGTADFPYLVTPAEAIQNEILSKGEGSVFPIFDNWASDQIKSAASQATVSLVFVNADSGEGFISVDGNEGDRKNLTLWKGGDELIQTVASYCNNTVVVIHSTGPVLVGEWNEHPNITAILWAGLPGQESGNSIADVLYGKVNPGGRSPFTWGRTAEDYGASILKEPNEGNGAPQVDFTEGIFTDYRAFDKADIKPIYEFGFGLSYTSFSYSDLNVEVPPWTLITDYRTKITSLLAQPMALHKNFFLLVGDPVEILVFMKYYIV

SEQ ID NO: 31 - protein, 777 aa, Exophiala dermatitidis
MHELYLWPFSDAVRAGTGSVMCSYNQINNSYGCANSYTMNHLLKNELDFQGFIMSDWQAQHSGVGTALAGLDMTMPGDTLFNTGRSYWGTNLTIAVINGTVPEWRVDDMATRIMAAYYYVGRDTHYTPTNFYAWSRNTYDKIHQVDPQSPIGLVNEHINVQDRHRDIVRQVGQASNVLLKNTGGLPLTGKERQVGIFGYDAGSNPWGANGCSNRGCDNGTLAMGYGSGTAEFPYLVTPEQAITQHVLTQTDGEVFAILDNYADAQIKSLASTADVALVFANAQSGEGFITIDGNTGDRNNLSLWLGADRLIHNVTKYNKNVIVVMHTVGPVNVSAWYDNENVTGIIWAGLPGQESGNAIVDALYGLINPGGKLPFTIGRNREDYGTDILYEPNNGQFNAPQSLFSEGVFIDYRHFDQYNIEPIYEFGFGLSYTTFEYSNLVITPGNPAPYTPTSGQTEPAPVLGNASTDPSQYVFPNGTITYRPYLYIYPYLNSTDLRASSDDTDYGLPTDQYVPPGATDGSPQPLLPAGGAPGGNAGLYEVVATVSATITNTGSVEGDEVAQLYVSLGEGEPPKVLRGFDRLTIAPGASTTFTANLTRRDVSVWDTVSQNWVQVSNPTIYVGTSSRKLPLSGVLSSSGGGSGAQSSSSASGGSGGGSSSGWGYGQSSTASGSAPAPVSQLSDGQPQVPTGRPVTQVSDGQPQAPTGNPVSQISDGQPQAPAHTGGAPVSQISDGQPQVPTGTGPAVTQISDGQPQNPTGGPAVSQLSDGQPRATTA

SEQ ID NO: 32 - protein, 860 aa, Aspergillus welwitschiae
MRFTLIEAVALTAVSLASADELAYSPPYYPSPWANGQGDWAEAYQRAVDIVSQMTLAEKVNLTTGTGWELELCVGQTGGVPRLGIPGMCAQDSPLGVRDSDYNSAFPAGVNVAATWDKNLAYLRGQAMGQEFSDKGADIQLGPAAGPLGRSPDGGRNWEGFSPDPALSGVLFAETIKGIQDAGVVATAKHYIAYEQEHFRQAPEAQGYGFNITESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTISVLNGTVPQWRVDDMAVRIMAAYYKVGRDRLWTPPNFSSWTRDEYGFKYYYVSEGPYEKVNQFVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLYTEPNNGNGAPQEDFVEGVFIDYRGFDKRNETPIYEFGYGLSYTTFNYSNLQVEVLSAPAYEPASGETEAAPTFGEVGNASDYLYPDGLQRITKFIYPWLNSTDLEASSGDASYGQDASDYLPEGATDGSAQPILPAGGGAGGNPRLYDELIRVSVTIKNTGKVAGDEVPQLYVSLGGPNEPKIVLRQFERITLQPSEETQWSTTLTRRDLANWNVETQDWEITSYPKMVFVGSSSRKLPLRASLPTVH

SEQ ID NO: 33 - protein, 860 aa, Aspergillus eucalypticola
MRFTLIEAVALTAVSLASADELAYSPPYYPSPWANGQGDWAQAYQRAVDIVSQMTLAEKVNLTTGTGWELELCVGQTGGVPRLGVPGMCLQDSPLGVRDSDYNSAFPAGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNQYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVVIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAQTFGEVGNASDYLYPDGLQRITKFIYPWLNSTDLEASAGDSSYGQDSSDYLPEGATDGSAQPILPAGGGPGGNPRLYDELIRVSVTIKNTGKVAGDEVPQLYVSLGGPNEPKIVLRQFERITLQPSEETTWNTTLTRRDLANWNVEKQDWEITSYPKMVFVGSSSRKLPLRASLPTVH

SEQ ID NO: 34 - protein, 838 aa, Aspergillus luchuensis
MRFTLIEAVALTAVSLASADELAYSPPYYPSPWANGQGDWAQAYQRAVDIVSQMTLAEKVNLTTGTGYVLVRLAGFPADYNSAFPSGMNVAATWDKNLAYLRGKAMGQEFSDKGADIQLGPAAGPLGRSPDGGRNWEGFSPDPALSGVLFAETIKGIQDAGVVATAKHYIAYEQEHFRQAPEAQGYGFNISESGSANLDDKTMHELYLWPFADAIRAGAGAVMCSYNQINNSYGCQNSYTLNKLLKAELGFQGFVMSDWAAHHAGVSGALAGLDMSMPGDVDYDSGTSYWGTNLTVSVLNGTVPQWRVDDMAVRIMAAYYKVGRDRLWTPPNFSSWTRDEYGYKYYYVSEGPYEKVNHYVNVQRNHSELIRRIGADSTVLLKNDGALPLTGKERLVALIGEDAGSNPYGANGCSDRGCDNGTLAMGWGSGTANFPYLVTPEQAISNEVLKNKNGVFTATDNWAIDQIEALAKTASVSLVFVNADSGEGYINVDGNLGDRRNLTLWRNGDNVIKAAASNCNNTIVIIHSVGPVLVNEWYDNPNVTAILWGGLPGQESGNSLADVLYGRVNPGAKSPFTWGKTREAYQDYLVTEPNNGNGAPQEDFVEGVFIDYRGFDKRNETPIYEFGYGLSYTTFNYSNLEVQVLSAPAYEPASGETEAAPTFGEVGNASNYLYPDGLQKITKFIYPWLNSTDLEASSGDASYGQDSSDYLPEGATDGSAQPILPAGGGPGGNPRLYDELIRVSVTIKNTGKVAGDEVPQLYVSLGGPNEPKIVLRQFERITLQPSEETKWSTTLTRRDLANWNVEKQDWEITSYPKMVFVGSSSRKLPLRASLPTVH

SEQ ID NO: 35 - protein, 860 aa, Aspergillus heteromorphus
MRFGWIEAAALTAASVVSADELAYSPPFYPSPWANGQGDWASAYERAVAIVSQMTLAEKVNLTTGTGWELEKCVGQTGGVPRLGIPGMCGQDSPLGVRDSDYNSAFPAGINIGATWDKNLAYLRGQAMGQEFSDKGADFQLGPVAGPLGRAPDGGRNWEGFSPDPALTGVLFAETIKGIQDAGVVATAKHFIGYEQEHFREAPEAQGYGYNITESGSANIDDKTMHELYLWPFADAVRAGVGAIMCSYNQINNSYACQNSYALNKLLKGELGFQGFVMSDWEAHHSGASAAMAGLDMSMPGDVVFDSGTSYWGTNLTIGVLNGTVPQWRVDDMAVRIMAAYYKVGRDRLWTPPNFSSWTRDEYGFKYYYSSEGPYEVVNHFVDVRRNHSDLIRRIGADSTVLLKNDGCALPLTGTERKVALIGEDAGSNPYGADGCSDRGCDNGTLAMGWGSGTTEYPYLVTPEQAIQAEVIQNGGTVFAVTDNWAISQMETLAAEATVSLVFVNADSGEGYIDVDGNMGDRNNLTLWGNGDNVIKAAASNCNNTIVIIHSVGPVLVNEWYDHPNVTAILWAGLPGQESGNSLADVLYGRVNPGAKSPFTWGKTRESYQDYLITEPNNGDGAPQEDFTEGVFIDYRGFDKRNETPIYEFGYGLSYTTFNYSQLQVQALNASAYTPASGETEAAPTLGEAGNASDYLYPSGLQRITAFIYPWLNSTDLEASSGDSSYGQSSDFLPEGATDGSAQPILPAGGGPGGNPALYDDLIQVSVTIKNTGDIAGDEIPQLYVSLGGPDEPRIVLRKFDRITLQPSEEYEWTTTLTRRDLSNWDVAAQDWIVTSYPKKVYVGSSSRKLPLRASLPTVQ

SEQ ID NO: 36 - protein, 860 aa, Aspergillus aculeatus
MKLSWLEAAALTAASVVSADELAFSPPFYPSPWANGQGEWAEAYQRAVAIVSQMTLDEKVNLTTGTGWELEKCVGQTGGVPRLNIGGMCLQDSPLGIRDSDYNSAFPAGVNVAATWDKNLAYLRGQAMGQEFSDKGIDVQLGPAAGPLGRSPDGGRNWEGFSPDPALTGVLFAETIKGIQDAGVVATAKHYILNEQEHFRQVAEAAGYGFNISDTISSNVDDKTIHEMYLWPFADAVRAGVGAIMCSYNQINNSYGCQNSYTLNKLLKAELGFQGFVMSDWGAHHSGVGSALAGLDMSMPGDITFDSATSFWGTNLTIAVLNGTVPQWRVDDMAVRIMAAYYKVGRDRLYQPPNFSSWTRDEYGFKYFYPQEGPYEKVNHFVNVQRNHSEVIRKLGADSTVLLKNNNALPLTGKERKVAILGEDAGSNSYGANGCSDRGCDNGTLAMAWGSGTAEFPYLVTPEQAIQAEVLKHKGSVYAITDNWALSQVETLAKQASVSLVFVNSDAGEGYISVDGNEGDRNNLTLWKNGDNLIKAAANNCNNTIVVIHSVGPVLVDEWYDHPNVTAILWAGLPGQESGNSLADVLYGRVNPGAKSPFTWGKTREAYGDYLVRELNNGNGAPQDDFSEGVFIDYRGFDKRNETPIYEFGHGLSYTTFNYSGLHIQVLNASSNAQVATETGAAPTFGQVGNASDYVYPEGLTRISKFIYPWLNSTDLKASSGDPYYGVDTAEHVPEGATDGSPQPVLPAGGGSGGNPRLYDELIRVSVTVKNTGRVAGDAVPQLYVSLGGPNEPKVVLRKFDRLTLKPSEETVWTTTLTRRDLSNWDVAAQDWVITSYPKKVHVGSSSRQLPLHAALPKVQ

SEQ ID NO: 37 - protein, 861 aa, Aspergillus caelatus
MKLGWFEVAALAAASVVSAQDDLAYSPPFYPSPWADGQGEWAEVYKRAVEIVSQMTLTEKVNLTTGTGWQLERCVGQTGSVPRLNIPSLCLQDSPLGIRFSDYNSAFPAGVNVAATWDKTLAYLRGQAMGEEFSDKGIDVQLGPAAGPLGAHPDGGRNWEGFSPDPALTGVLFAETIKGIQDAGVIATAKHYILNEQEHFRQQPEAAGYGFNVSDSLSSNVDDKTIHELYLWPFADAVRAGVGAVMCSYNQINNSYGCENSETLNKLLKAELGFQGFVMSDWTAHHSGVGAALAGMDMSMPGDVTFDSGTSFWGANLTIGVLNGTIPQWRVDDMAVRIMAAYYKVGRDTKYTPPNFSSWTRDEYGFAHNHVSEGAYERVNEFVDVQRDHADLIRRIGAESTVLLKNKGALPLSRKEKLVALLGEDAGSNPWGANGCDDRGCDNGTLAMAWGSGTANFPYLVTPEQAIQNEVLQGRGNVFAVTDSWALDKIAAAARQASVSLVFVNSDSGEGFLSVDGNEGDRNNITLWKNGDNVVKTAANNCNNTVVIIHSVGPVLINEWYDHPNVTGILWAGLPGQESGNSIADVLYGRVNPGAKSPFTWGKTRESYGSPLVNEANNGNGAPQSDFTQGVFIDYRHFDKFNETPIYEFGYGLSYTTFELSDLHVQPLNASQYTPTSGMTEAAKNFGETGDASEYVYPEGLERIHEFIYPWINSTDLKASSDDSNYGWEDSKYIPEGATDGSAQPRLPASGGAGGNPGLYEDLFRVSVKVKNTGKVAGDEVPQLYVSLGGPNEPKVVLRKFERIHLAPSQEVVWTTTLTRRDLANWDVSAQDWTVTPYPKTIYVGNSSRKLPLQASLPKAR

SEQ ID NO: 38 - protein, 854 aa, Madurella mycetomatis
MANLVLALLLFLSALTSAATDTPFLGYPSPWATNITDPTWAAAYAQAIAFVANLTLTEKVNLTTGTGWEADRCIGATGGIPRLGFRPFCLMDGPLGVRYTDHNSAFPAGVNTAATFSRRLMRLRGEAMGAEFRGKGIDVMLGPVAGALGRVPQGGRNWEGFSPDPYLTGVAMAETIQGIQSRGVVACAKHYILNEQEHFRGSIDVRIDDRTMHELYLWPFADAVRAGVGSVMCSYNKINGTYACENEWTTNYLLKNELGFQGFVLSDWGAQHNTLGSALGGLDMAMPGDGGPPPYRAWWGGALTEAVLRGDVPQWRLDDMAVRIMAAYFRVHTGNYTSRPDINFSAWTNSTVGPLYPAANQSYTVVNEFVDVQSDHASLIREIGAKSVVLLKNAYDLLPLRQPGPPIIAVIGDDAQDHPLGPNACPERGCLNGTLAMGYGSGTANFPYLVSPLTALTEQARADNTTLLYAPSNWDLDAAITTARNASIAIVFAAATSGENFITVDGNAGDRNNLTLWANGDALIKAVASINPNTIVVLHTPGPVILDYAEEHPNISAILWAGLPGQESGNALVDVLYGKVNPQGRSPFTWGDSVEEYGAQLMFEAENPRAPVQSFDEGVFIDYRKFSTFGGKLTYPFGFGLSYTKFRYSGLSVVRKEEVEGRFEPAEGLTGPAPTFGVVEAELGVHTAPEGFTRISPYVYPWLNNSESLVTGNSTGASEFPAAARNGSAQPVLPASGAPGGNPGLYEVLYTITASIENVGEVAGTEIPQLYVQLGGEENPFGVLRGFDEVELEPGETKNVTFELTRRDVSNWNTSTQNWEITDREKVVFVGSSVRDIRLNASLPAPVGMAWRHG

SEQ ID NO: 39 - protein, 975 aa, Aureobasidium pullulans
MSQSQDYELTPRESPRQSLDHPEDYRLSTSSRDSISSSINELDPLKTPNRPYKDDVSQTHDSVFVTRRRSTWRRYLLPSRMCCMMVLLFTAALVMLLSAGGIWVYKTEPEGGQSDPWYPAPRGGSVTAWEEAYKKAAALVGQMSVVEKVNITTGTGWEMGMCVGNTGPVERLGFPSLCLQDGPLGLRFADNITAFPAGITLGTTWNKELIYLKGRAHGREARGKGVHIALGPSMGPFGRLPAGGRNWEGFGSDPVLQGLAAAAMIRGIQEEGIIATAKHYIANEQEHFRQSWEWGTPNAISSNLDDRTLHEIYAWPFAESVKAGVGSVMCSYNLVNQSYACQNSKLLNGILKDELGFQGFIQSDWLAQRSGVASALAGLDMSMPGDGLRWMDGQSVWGGELTKAVFNGSVPMERLNDMVLRVVAAWYQFGQDDKKKWPAEKDGGGPNFSSWTNDEIGLLHPGSNDTTKGVVNKFVNVQGEGDDSHGKLARQIAAEGTVLLKNDDHVLPLNRDGKKLTKNGDKLRVAIFGEDAYNNGKGPNACADRACNEGTLAQGWGSGTVEFPYLVAPAEGIHLGFDVESVTLSDYPSNYEDLDKHPSRAADQDLCFVFINSDGGEGYVAWKDVKGDRNDLYAQKGGDELVQKVAAKCGGPTIVVVHAVGPVILEKWIDLPGVKAVLYANLPGQESGNALASIVFGDINPSGRLPYTIAKNEDDYGPTSKILYRPNAVIPQQNFSEGLYVDYRYFDKHDIEPRYEFGFGLSYTTFHLSSLFIHPLATEYSTLPAKRPESITPPHLDVSIPDAREALWPSNFIALKKYIYPYINSVDDIKTGKYPYPVGYEIAQPLSQAGGAEGGNPDLYTPLAVVQASLTNTGSLPGDCVVQLYISYPSTPIIDSLGNTVDFPVRVLRAFDKLFVTPEKRVEVSLQLTRKDLSFWDVGLQNWVLPMGDFEVQLGFSSRDIQQKGDILSFSRLLL

SEQ ID NO: 40 - protein, 1220 aa, Fusarium oxysporum
MALIICFYYPISLGEAPDSDHTLKEKILGLGLKSAVILAGALVCLFLALQWGGTKHPWSDSRVWGCLVGFGLLLTLFVYIQIRQGEAALIRPRIISQRSVFLGCLFSALYQGAMTTQSYHLPFYFQAVKGVDPQSSGVDILPHGVTVTIATLITGSIITWLGYYVPFMWAGSAIFTIGAGLLYTISQNTPLARWFGYEVLAGAGFGIAIQIPIFAVQVVLVAGDIPLGTVLIILSQALGGSVGLSISQNVFQNSLRQRLNTIADIDIQVVIAAGGTDLEHVVSADSLAHVRDAFRYGISNAFLVSTALGGVAVLASIGMERKKIKSKKEGVDLYKTARPFDVSVFSSRGSIDTKMTLAKVAFTLANLVAVGSSAKNVADGFVAAPYYPAPYGGWDEAWADSYARAKKMVDSMTLAEKTNITAGTGLFMADRVGFPQLCLNDAANGVRLADNVTVFPDGITAGATFDKKLMYERGVAIGKEARGKGVNVWLGPSVGPIGRKPKGGRNWEGFGADPSLQGIGARETIKGVQEQGVIATVKHLIGNEQEMYRRQTTDVVSPAYSANIDDRTMHELYLWPFAEAVKVGVGATMTAYNRVNGTISSEHSYLINALLKEELGFQGFVMTDWLSQITGVQSAIAGMDMSMPGDTIIPLFGNSLWMYELTRSALNGSVPMSRLNDMATRIVATWYQFEQDKDFPSVNFDTNTYNRVGPLYPAAWPNSPSGVVNQYVQVQDDHDEIARQIAQDAITLLKNDEKLLPLSTKSSLKVFGTGAQTNPDGANACVDRSCNKGTLGQGWGSGTVDYMYLDDPIGAIKKAAGDVTFYNTDKFPSVPSPSDDDVAIVFVTSDSGENQYTVEGNNGDRNADKLNVWHNGDALIKAAAAKYKNVVVVIHTVGPVLVDQWIDLPSVKSVLVAHLPGQEAGKSLTNILFGDASPCGHLPYSITKKEDDMPESVTKLIDSGFIDAPQDTYSEGLFIDYRWLNKEKIQPRYAFGHGLSYTNFTYTNATIKRGTQLSQYPPKRPAKGKVLDYSQDIPDYKEAIKPSSFKTVWRYLYSWLSESDAKSAAAKAETSKYPYPDGYSTAQKTALPKAGGVSGGNPALWDEAYTLSVVVTNAGSKFSGKASVQAYVQFPSVAGYETPVIQLRDFEKTKVLEPGSSETVQLTLTRKDLSVWDVKAQDWLVLDGEFKIWLGSASDKLDAVCFTDDLGCEHDVKGPVSYDS

SEQ ID NO: 41 - DNA, 1437 bp, Cellulomonas wangleii
  source 1..1437: mol_type = genomic DNA
atgacccccccaccccagctccgacgcgggcgccccctggtcgcacgcggcgcctccgccatcgcgatcgtcgcgctcgcggtgacggccgccgcggccatcccggcgcaggccgccggctcgacgctccaggaggccgccgcccagagcggccgctacttcggcaccgcgatcgccgcgaacaagctcagcgactcgacctacgtgaccatcgcgaaccgtgagttcaacatgatcacggccgagaacgagatgaagatggacgcgacggagccgaaccagaaccagttcaactacagccagggcgaccggatcctcaactgggccaagcagaacggcaagcaggtccgcgggcacgcgctcgcgtggtactcccagcagccggagtggatgaagcgcatggagggctccgcgctgcgcagcgcgatggtcaaccacgtcacccaggtggcgacgcactacaagggtcagatctacgcctgggacgtcgtgaacgaggcgttcgccgacggcagctccggcggacgccgcgactccaacctggagcgcaccggcagcgactggatcgaggtggccttccgcgccgcgcgcgccgccgacccggccgcgatcctctgctacaacgactacaacatcgacgactggtcgcacgccaagacccagggcgtctaccggatggtccgtgacttcaagcagcgcggcgtcccgatcgactgcgtcggcctgcagtcgcacttcaacccgcagagcccggtcccggccaactaccagacgacgatcgagagcttcgccgccctcggcgtcgacgtgcagatcaccgagctcgacatcgagggctccggctcggcgcaggccgagaacttccgcaaggtgacccaggcctgcctcaacgtcgcccgctgcaccggcatcacggtgtggggcgtgcgtgacacggactcgtggcgcgcctcgggcaccccgctgctgttcgacggcaacggcaacaagaagcaggcgtacacctcggtgctcaacacgctgaacgcggcgagcccgaccacgccgccgcccaccacgccgccgccgacgaccccgccgccggtgacgcccccgcccacgaccccgccgcccaccacgccgccgccgacgaccccgccgccgacgaccccgccgccggtgacgggcagctgctccgccgcactgaccatcgccaacgcgtggcccggtggctaccagggcacggtcacggtgacggccggctcgtcgtccctcaacgggtggcgggtgaccctgccgggtggcgtctccacgaacaacgtgtggaacggcgtggtctccggcaacgtcgtgagcaacgcgccctacaacggctccgtcggggccgggcagtcgacgaccttcgggttcatcgggaacggcaacgccccgtcgccgacctccctcacctgctcctga

SEQ ID NO: 42 - DNA, 1419 bp, Cellulomonas shaoxiangyii
  source 1..1419: mol_type = genomic DNA
atgctccgacgagggagcaggcgcggccccggcgctcgaggcgcagccgtcctcgccgccctgaccctcgcggcgaccgccgcggccgccatcccggctcaggccgccggctcgacgctgcaggacgcggccaacgaccgcggccgctacttcggcacggcgatcgccgcgaaccgcctcagcgactcgacctactcgaccatcgcgaaccgtgagttcgacatgatcacggccgagaacgagatgaagatggatgcgacggagccgtcgcagaaccagttcaactacaccaacggcgaccgcatcgtgaactgggcgctccagaacggcaagcaggtccgcgggcacgcgctggcgtggcactcgcagcagccgggctggatgcagaacatgtccggcacggcgctgcgcaacgccatgctcaaccacgtcacgcaggtcgccacgcactaccgcggcaagatctacgcctgggacgtcgtgaacgaggccttcgccgacggctcgtccggcgcccgccgcgactccaacctgcagcgcaccggcaacgactggatcgaggcggccttccgcgccgcccgggccgccgaccccggcgcgaagctctgctacaacgactacaacaccgacgactggacgcacgcgaagacgcaggccgtgtacaacatggtccgcgacttcaaggcgcgcggcgtcccgatcgactgcgtcggcctgcagtcgcacttcaacgcgcagagcccggtgccgagcaactaccagacgaccctgtcgagcttcgccgcgctcggcgtggacgtgcagatcaccgagctcgacatcgagggctcgggctccgcgcaggcggagagctaccggcgcgtcgtccaggcgtgcctcaacgtctcccgctgcaccggcatcacggtgtggggcgtgcgggacaccgactcgtggcgcgcctcgggcacgccgctgctcttcgacggccagggcaacaagaaggcggcgtacaccgcggtcctcgacacgctgaacagcggacccgggaccaacccgacgacgcctccgcccaccagccccccgccgacgaccccgccgccggtcaccccgccgccgacgacgcccccgcccacgaccccgccgccggtcaccccgccgcccagcggcacctgctccgcggcactgacgatcgcgaacagctggggcggcggctaccaggccaccgtcacggtgcgggccggttcgtccgctctcaacggatggcgggtcaccctgcccggcggcgtcacgacgtcgaacctgtggaacggcgtcctcgccggcagcgtcgtgacgaacgcgccgtacaacggctccgtcggagccggccagtcgacgtccttcgggttcatcggcaacggcagcgcacccgccgcgggagcgctgagctgcagctga

SEQ ID NO: 43 - DNA, 1446 bp, Micromonospora sp. WMMA1998
  source 1..1446: mol_type = genomic DNA
atggacaaggtgctcgcccgcggcagcgggagcccgaccgcccgataccggccgcgtgccgccctgctgtcggccgccgtcggcgcggcgctggtcgcggccacggtcgcgatggcgaccagcgccagcgccgggaccaccctgggcgcggcggcggcggaacagggccgctacttcggcaccgcggtggccgcgaacaagctgtcggacgccacgtacgtcggcatcctgaaccgcgagttcactatggtcacccccgagaacgagatgaagtgggacgccaccgagccgtcccagggccagttcagctacaccaacgccgaccggatcgtcgcccacgcccaggccaacggcatgcgggtacgcggccacgcgctggcctggcactcgcaacagcccggctgggcgcagaacctgtccggcagcgcgctgcgccaggcgatggtcaaccacatcacccaggtcgccacccactacaagggcaagatctacgcctgggacgtggtcaacgaggcgttcgacgacggcagcggcggccggcgcgactccaacctccagcgcaccggcaacgactggatcgaggtggcgttccgcaccgcgcgcgccgccgacccgggcgccaagctctgctacaacgactacaacaccgacaactggacctgggccaagacccagggcgtctacaacctggtcaaggacttcaaggcccggggcgtgccgatcgactgcgtcggcctgcagtcgcacttcaacagcggctcgccgtacccgagcaactaccgcaccacgttgcagaacttcgccgccctcggcgtcgacgtgcagatcaccgaactcgacatcgagggttcgggcagcgcccaggccaccacgtacgccaacgtggtcaaggactgcctggccgtgtcgcggtgcaccggcatcaccgtctggggaatccgggacagcgactcctggcgggccggcggcaccccgctgctcttcgacggcaacggcaacaagaaggcggcctacaccgccgcgctcgacgcgctcaacgccggcggcacgccgccgccgaccaccacgccgccgaccaccacgcccccgaccacgactccgccgacgaccacgccgcccaccaccgcgccgcccacgactcccccgcccaccggcggcgggtgcaccgcgtcgctcaccaccaaccagtggcccggcgggttcgtcaccaccgtgcgcgtcaccgccggcgccggcgcgctcaacggctggacggtgacgctcaccgtgccgtccggctcggcggtcaccaacacgtggagcgcccaggccagcggcgccagtggcgcggtgacgttccgcaacgtcgcctacaacggccaggtcggtgccggcggcagcaccgagttcggcttccaggggaccggcacggccccctccggcacgcccacctgcgcggcgggctga

SEQ ID NO: 44 - DNA, 1425 bp, Xylanimonas cellulosilytica
  source 1..1425: mol_type = genomic DNA
atgaaccatcagcaccaccgtcgtaggttgcgcagcgtcgcagccgtcgcgctcgcctccctggtcgtcgccacgggagtggtgaccgcccaggcggccggatccacgctccaggaagcggcgggcagccgctacttcggcacggccatcgcggccaacaagctctcggactcgacctactcgaccatcgcgaaccgtgagttcgacatgatcacggccgagaacgagatgaagatggacgccaccgagccgtctcagggcagcttcaacttcaccaacgccgacaggatcgtcgactgggccaccgccaacggcaagcggatgcgcggtcacgccctcgcgtggcactcgcagcagccggggtggatgcagaacatgtcgggcaccgcgctgcgcacggcgatgctcaaccacgtcaccgaggtcgctgcgcactacaagggcaagatctacgcctgggacgtcgtcaacgaggccttcgccgacgggtcctcgggcgcccgccgcgactcgaacctgcagcgcacgggcgacgactggatcgaggccgcgttcagggccgcccgggccgccgacccgtccgccaagctctgttacaacgactacaacacggacaactggaactgggagaagacccaggccgtgtacgccatggtcaaggacttcaaggaacgcggcgtgccgatcgactgcgtcgggctccagtcccacttcaactcgggcagcccctacccgagcaactaccgcacgacgctgcagaacttcgcggcgctcggcgtcgacgtccagatcaccgagctggacatcgagggctcgggcagcacgcaggccgacacgtacgccaaggtggtcgccgactgcctcgcggtgagccgctgcaccggcatcacggtgtggggcgtgcgcgactccgactcgtggcgcgcgagcggtacgccgctgctgttcgacgcgtcgggcaacaagaaggccgcctacacgtcggtgctgaacaccctcaacggcggtgcgacgccgacgccgacgccgacgccgacgccgacgccgaccccgactcccacgccgactccgaccccgacgccgacgccgactcccacgccgacgccgacgccgacgccgactcccacgccgacgccgacgtccggtccgtgcaccgccacgatgacgatcacgaactcgtggcagggcggcttccagggtgaggtcacggtcaaggcgggcagtgcccgcacgtcctggtcgacgtcgttcacctcggccgccacggtccaagtctggaacggggtgcactcgacctccggctcggtgcacacggtgtcgaaccagccgtacaacggcacgctggccgccggtgcgtcgacgacgtacggcttcacggccaccgggacggcgccgagcagcccgcccgcggtgacctgctcgtga

SEQ ID NO: 45 - DNA, 1443 bp, Verrucosispora sp. WMMD573
  source 1..1443: mol_type = genomic DNA
atgaacaacgcccacgcccgcgccagcgggcggccggctacccgactgcgaccgcgcgccgcgctggtctcggccgcggtcggtctcgccctcgtcgccaccagcatggtggtgacctccagcgcccaggccgccgagtccaccctcggtgcggccgcggcccagtccggccggtacttcggtgccgcggtggcggcgaacaagctgtccgactccacctacgtcggcatcctgaaccgcgagttcaactcggtcacggccgagaacgagatgaagatcgacgcgctcgagccgcagcagaacaacttcacgttcggcaacgcggaccggatcgtcaaccacgccctgtcccggggctggaaggtccgggggcacaccctggcctggcactcgcagcagcctggctggatgcagcagatggaaggcacggcgctgcgtaacgcgatgctgaaccacgtcacccgggtcgcgacctactaccgcggcaagatcgactcgtgggacgtggtgaacgaggcgttcgacgacggcaacaacggcgcgcgccgtaactcgaacctgcagcgcaccggtaacgactggatcgaggccgcgttccgggccgcccgggccgccgacccgggcgccaagctctgctacaacgactacaacaccgacaactggacctgggccaagactcaggccgtctaccggatggtgcaggacttcaagcagcgtggcgtgccgatcgactgcgtcggcctccagtcgcacttcaacagcggctcgccgtatccgagcaactaccgcaccacgctgtccagcttcgccgcgctcggcgtggacgtgcagatcaccgagctggacatcgaaggctccggcagcacccaggccaacacctaccgcaacgtggtcaacgactgcctcgcggtgccccgctgcaacggcatcaccgtgtggggaatccgggacaccgactcctggcgttccggtggcaccccgctgctcttcgacggcaacggcaacaagaagcaggcgtacgacgcgacgctccaggcgctgaactcgggcggcaccaccccgccgcccacgaccccgccgccgcccacgaccccgccgcccaccacgccgccgcccacgaccccgccgccgaccaccccgccgcccggcgggtccggctgcacggcaacggtgtccctgaaccagtggaacggtggtttcgtcgccacgatccgcgtcaccgccggttccgcgagactgaacggctggtcggtgggcatcgggattccgggcggtagctcggtgaccaacacctggaacgcccaggccagcggcaacagcggcaacgtgacgttccgcaacgtcagctacaacggcaccgtgaacgccggtgccaccacggagttcggcttccagggcaccggtaccggtccatccggtacccccacctgcaccggtagctga

SEQ ID NO: 46 - DNA, 2043 bp, Saccharothrix syringae
  source 1..2043: mol_type = genomic DNA
atgtccagaactgctgtcacagctgccggcaggtcggaggcacgaggccggtcgcggcgcgccgcggtggtggtcggtgcgatcgggttgctgagcgcggcggccgtggtgctgccgaacgtggccaccgccggcaccacgctgggcgcgtccgccgcggagagcgggcgctacttcggcacggcggtcgcggccaacaagctctcggactcgacctacgtgggcatcctcaaccgtgagttcgacatggtcacggctgagaacgagatgaagatggacgccacggagccgaaccagaaccagttctcgttcggcaacggtgaccggatcgtcaaccacgcccgcaaccagggcaagcgggtccgtggccacgcgttggcgtggcactcgcagcagccgggctggatgcagaacatgtccggcacggcgttgcgcaacgcgatgctcaaccacgtcacccaggtcgccacgtactacaagggcaagatctacgcctgggacgtggtcaacgaggcgtacgccgacggcagctccggcggtcggcgtgactcgaacctccagcggaccggcaacgactggatcgaggccgccttccgggccgcccgcgccgccgacccgaacgcgaagctgtgctacaacgactacaacaccgacaactggtcgcacgccaagacccagggcgtgtaccggatggtgcaggacttcaagtcgcgcggtgtgccgatcgactgcgtcggcttccaggcgcacttcaacagcggcaacccggtgccgtcgaactaccacaccaccctgcagaacttcgccgacctcggcgtggacgtccagatcaccgagctggacatcgagggctccggcagcacccaggcccagcagtaccagggcgtggtccaggcgtgcctcgcggtgacccgctgcaccggcatcacggtgtggggcatccgcgacagcgactcgtggcgttcctcgggcaccccgctgctgttcgacggctcgggcaacaagaaggccgcctacacctcggtcctcaacgccctcaacgcgggcagcaccgcgccgccgtccaccaccacgacgacgaccccgccgaacacctcggactgccgcgccggctacgtcggcctgacctacgacgacggccccaacggcagcaccaccacgcagctgctcaacgcgctgcggtcggccggcctgcgcgccacgttcttcaaccagggcaaccgggtccagcagaaccccggcctggccaaggcgcagcgcgacgccggcatgtgggtcggcaaccacagctggagccacccgcacatgacccagctgagccagtcgcagatggcctcggagatctcccagacccagcaggccatccagtcggccaccggtgaggcgccgaagctgttccgcccgccctacggcgagaccaacagcacgctcaagtcggtcgaggcccagtacggcctgaccgaggtgctgtggagcgtcgactcgcaggactggaacaacgccagcaccgcgcagatcgtgcaggccgcgtcgaccctgcagaacggcggcgtgatcctgatgcacgacggctaccagacgacgatcaacgccatcccccagatcgcggccaacctcgccagccgcggcctgtgcgccggcatgatctccacgtcgaccggccaggccgtcgcgccgaacgacaacccgcccaccaccggcccgacgaccaccaccacgacgacgtcccagcagcccggcggctcgtgcaccgcgacctaccggaccacccagcagtggggcgaccgcttcaacggcgaggtgaccgtccgggcgggcgcctccgcgatcaccagctggacggccaccgtcacggtgacctccccgcagaaggtgtcggccacctggaacggcacgccgagctgggactccagcggcaacgtcatgaccatgaagcccaacggcaacggaaacctcgccgcgggcgcgagcacgacgttcggcttcaccgtgatgaccaacggccagtgggcggcgccgaccgtctcctgccgcacgccgtga

SEQ ID NO: 47 - DNA, 1410 bp, Cellulomonas palmilytica
  source 1..1410: mol_type = genomic DNA
atgaccccctcatccatgtccagacgcgcgcgcttcgcggccgcgctcgcagtcgtcacgctcggggcgaccatcgctgcgacgatccccgcccaggccgcgggaagcacgctgaaggatgccgcagcgcagagcggccggtacttcggcaccgcgatcgccggcttcaagctgagcgactcgacgtactcgtccatcgcgaaccgtgagttcaacatgatcacggccgagaacgagatgaagatggacgcgacggagccgtcgcagaacaacttcaacttctccagcggcgaccagatcctcaactgggccgtccagaacggcaagcgcgtgcgcggccacgccctggcctggcactcgcagcagcccagctggatgcagggcatgtccggcagcgcgctgcgcagcgccatgctcaaccacgtcaccaaggtcgccgagcactacaagggcaaggtctatgcctgggacgtcgtgaacgaggcgttcgacgacagcaacggcgggcgccgcgactccaacctgcagcgcaccggcaacgactggatcgaggccgcgttcaaggccgcccgcgccgccgacccgaacgccaagctctgctacaacgactacaacaccgacaactggacctgggccaagacgcagggcgtctacaacatggtcaaggacttcaaggcccgtggcgtgccgatcgactgcgtcggcttccagtcgcacttcaacgcgcagagcgcctacaacagcaactaccgcaccacgctgtcgagcttcgcggcgctgggtgtcgaggtccagatcaccgagctcgacatcgagggctccggctcgcagcaggcggacacgtaccgtcgcgtcgtcgaggactgcctcgccgtcaaggcctgcaccggcatcacggtgtggggcgttcgtgactccgactcgtggcgctcctacggcacgccgctgctgttcgacaacaacggcggcaagaaggccgcgtacacctcggtcctcaacgcgctgaacgccgcggacccgacggacccgacgaccgaccccacgcaggacccgacggacgacccgacccaggacccgacggacgacccgacggacgacccgacggacccggagacgacgaacccgccggacccgaccggtaagtgctccgcggcgctcacgatcgtgaactcctggccgggcggctaccaggccacggtgacggtcaaggccggctcctcgtcgatcaacggctggcgggtcaccctgcccagcagtgtgaacacgaacaacctgtggaacggcgtcctgtccggtggcgtggtgaccaacgccccgtacaacggctcggtcggtgccggccagtcgacgaccttcgggttcgtcggcaacggcagcgccccgggcgcaggcaacctgacctgcgcctga

SEQ ID NO: 48 - DNA, 1374 bp, Micromonospora carbonacea
  source 1..1374: mol_type = genomic DNA
atgagacgcaagagagccctcctgacgacggtgaccctcgcggtcaccggcgcactcaccgccggcgtgctggtgacgatggcccccgccgccagcgccgggacgaccctgcgggctgccgcggccgagaagggccgctacttcggcgccgcggtcgcgacgggcaaactctccaacagcacgtacacgacgattctcaaccgcgagttcaacagcgtcgtggccgagaacgagatgaagtgggacgccaccgagccgcagcagggccagttcaactacagcggcggcgaccgcctcgtcagccacgcccgggccaacgggatgagcgtgcggggccacgccctgctctggcaccagcaggagccgggctgggcgcagggcatgtccggcagcgccctgcgcagcgcgatgatcaaccacgtcacccaggtcgccacccacttcaaggggcagatctacgcctgggacgtggtgaacgaggcgttcgccgacggcaacagcggcggccggcgtgactcgaacctccagcgcaccggcaacgactggatcgaggcggcgttccgcgccgcgcgggccgccgacccgggcgcgaagctctgctacaacgactacaacaccgacggggtcaacgcgaagtcgaccggcatctacaacatggtgcgcgacttcaagtcccggggcgtgccgatcgactgcgtcggcttccagtcccacctgggcaccacgctggccagcgactaccaggccaacctccagcgcttcgccgacctcggcgtcgacgtgcagatcaccgagctggacgtcatgaccggcggcaaccaggcgaacatcttcggcgcggtgacccgggcgtgcatgaacgtgtcgcgctgcaccggcatcacggtgtggggcgtgcgggactgcgactcgtggcgggggtccgacaacgccctgctgttcgactgcaacggcaacaagaagccggcgtacgactccgtcctcaacgccctcaatgccggcaccggcatccccaacccgacgaccaccccgccgaaccccaccaccactccgccgaacccgacgactccgccgccgggtggggccgggtgttcggcgacggtgtcggcgaattcgtggacgggtggtttcgtggccacggtgaaggtgaccgctggttctggtggtacccggggttggaacgtgagtgtgacgttgccgggtggtacgagtgtcacgggtacgtggtcggcgacggccagtggtagttcggggacggtgcggttcgccaacgtggactacaacggtcagctcgccgccggtcaggtgaccgagttcgggttccagggcaacggcaccgcgcccacccagacccccacctgcaccgccagctga

SEQ ID NO: 49 - protein, 469 aa, Cellulomonas palmilytica
MTPSSMSRRARFAAALAVVTLGATIAATIPAQAAGSTLKDAAAQSGRYFGTAIAGFKLSDSTYSSIANREFNMITAENEMKMDATEPSQNNFNFSSGDQILNWAVQNGKRVRGHALAWHSQQPSWMQGMSGSALRSAMLNHVTKVAEHYKGKVYAWDVVNEAFDDSNGGRRDSNLQRTGNDWIEAAFKAARAADPNAKLCYNDYNTDNWTWAKTQGVYNMVKDFKARGVPIDCVGFQSHFNAQSAYNSNYRTTLSSFAALGVEVQITELDIEGSGSQQADTYRRVVEDCLAVKACTGITVWGVRDSDSWRSYGTPLLFDNNGGKKAAYTSVLNALNAADPTDPTTDPTQDPTDDPTQDPTDDPTDDPTDPETTNPPDPTGKCSAALTIVNSWPGGYQATVTVKAGSSSINGWRVTLPSSVNTNNLWNGVLSGGVVTNAPYNGSVGAGQSTTFGFVGNGSAPGAGNLTCA

SEQ ID NO: 50 - protein, 437 aa, Promicromonospora iranensis
MTRTLTSPRHRRSLRAISLATLTAVVLAGGMAATTAQAAGSTLQAAATEKGRYFGTAIAANKLSDSTYTTIANREFNMVTAENEMKIDALEPNQNQFNWTNGDRIVSWARSNGKQVRGHTLAWHSQQPGWMQNMSGTALRNAMLNHVTQVATHYRGQIHSWDVVNEAFQDGSSGARRDSNLQRTGNDWIEAAFRAARAADPNAKLCYNDYNTDDWTHAKTQAVYNLVRDFKARGVPIDCVGLQSHFNAQSPVPSNYQTTISSFAALGVDVQITELDIEGSGSAQADNYRKVVQACLAVSRCTGISVWGVRDTDSWRASGTPLLFDGNGNKKPAYTATLDTLNGGTSNPQPGGCTVAVTRSTDWSDRFNVSLAVSGSSTWRVSIQLQGGQTLQNSWNATVSGSSGTLTATPNGAGNNFGITVYKNGNTNLPTATCSTT

SEQ ID NO: 51 - protein, 480 aa, Micromonospora ferruginea
MDKVFARTSHSTAARPRTRAAVVSMLAGAAAVAATVAVATSASAGTTLGASAAEQGRYFGTAVAANKLSDATYVGILNREFNMVTPENEMKWDATEPSQNQFTYSSSDRIVAHAQANGMRVRGHALAWHSQQPGWAQSLSGTALRNAMVNHITQVATHFKGKIYAWDVVNEAFDDGNGGRRDSNLQRTGNDWIEVAFRTARAADPGAKLCYNDYNTDNWTWAKTQGVYNMVKDFKARGVPIDCVGFQSHFNSGSPYPGNYRTTLQNFADLGVDVQITELDIEGSGSAQATTYGNVVKDCLAVARCNGITVWGIRDSDSWRASGTPLLFDGSGNKKAAYTSTLNALNAGGTTPPTSTPPTSTPPTSTPPTTPPPTTTPPTTPPPAGACTASLTTNQWQGGFVTTVRVTAGGSALNGWSVSLTLPSGSSVTNTWSAQASGAGGAVTFRNVDYNRQVGAGGSTEFGFQGTGTAPSGTVSCTAG

SEQ ID NO: 52 - protein, 444 aa, Actinoplanes friuliensis
MLSAASAGVVLAASIAVVGTAEAASTLGASAAQTGRYYGAAIAAGRLGDSTYTRILNTEFNSVTPENEMKWDATEPSQGRFTYTNGDRILNQGLSNGSKVRGHALLWHAQQPGWAQALSGSALRNAAINHVTQVATHYKGKIYAWDVVNEAFADGGSGGRRDSNLQRTGNDWIEAAFRAARAADPAAKLCYNDYNTDGINAKSTGIFNMVRDFKSRGVPIDCVGFQSHLGTGIDSTYQANLKRFADLGVDVQITELDIEQGGNQANIYSTVTKACLAVSRCTGITVWGIRDTDSWRTGANPLLFDGSGNKKPAYTAVLNALNAGGTTNPTTPPVTSPPASPTTPPPSGSGCTATVSLNSWSGGYVATVKVTAGSSAVTGWTVSATLPSGGALTGVWSATNTGTTGAVSFRNVEYNGRIPAGGSTEFGFQGTGTGPSAAPACRVG

SEQ ID NO: 53 - protein, 759 aa, Actinoplanes ianthinogenes
MIVAGIAGLVAAGLGVVYTQTADAATTLSASANQTGRYFGTAIPVSKLGDSAYTTILKTEFNAVTPENEMKWDATEPSQGSFNYTNGDKILNQGRAQGAKVRGHALLWHSQQPTWAQSLSGSALRTAAINHVTQVATHYKGKIYAWDVVNEAFADGGSGGRRDSNLQRTGNDWIEAAFKAARAADPAAKLCYNDYNTDGVNAKSTGVYTMVKDFKARGVPIDCVGFQSHLGTTVPADYQANLQRFADLGVDVQITELDVAQGGNQANIYASVTKACLAVSRCTGITVWGIRDSDSWRTGENPLLFDNSGTKKAAYTSVLNALNAGGTKPATSATTTPPGTAAPSAGAGCTAKLTAGEVWGDRYNSTITVSGASTWTVVVTITAPQKVSTVWNGTATYGGGSGQIMTVKPNGNGNSFGFTTMNNGNSTARPTITSCVAGGSTTSATAAPTTVPASAPSSCALPSKYRWTSTGALATPKSGWASLKDFTVVPYNGKHLVYATTHDTGSSWGSMNFSPFTNFSDMASAGQNAMSSGAVAPTLFYFAPKKIWVLTYQWGGSAFSYRTSSDPTNANGWSAAKPLFTGSISGSGTGPIDQTIIGDGTNMYLFFAGDNGKIYRASMPIGNFPGNFGSSYTTIMSDTTNNLFEAPQVYKVQGQNQYLMIVEAIGSNGRYFRSFTATSLSGSWTPQAATESNPFAGKANSGATWTNDISHGELVRVTADQTMTVDPCHLQLLYQGRSPSSGGDYGLLPYRPGVLTLQR

SEQ ID NO: 54 - protein, 475 aa, Antribacter gilvus
MKDLSPHRAGKFPWRSLAGAAGALAVAASLAVGVSTSAQAAGTTLQAAAAESGRYFGTAIAANKLSDSTYTTIANREFNMITAENEMKLDATEPSQNQFNYTNGDRIVNWATSNGKQVRGHTLAWHSQQPGWMQNMSGTALRSAMLNHVTQVATHYRGKIHSWDVVNEAFQDGSSGARRDSNLQRTGNDWIEAAFRAARAADPNAKLCYNDYNTDDWTHAKTQAVYNLVRDFKARGVPIDCVGLQSHFNSQSPVPSNYQTTLSSFAALGVDVQITELDIEGSGTAQADNYRRVVQACLAVSRCTGITVWGVRDTDSWRSSGTPLLFDGSGNKKAAYTSTLNALNAGGTTNPPTTPPPTTPPPTTPPPTTAPPTTPPPTQPGACTAAYRLVNSWQGGFQAEVVVTAGSARTGWTTSFTLPGGTSISQLWSGTLTSSGSTHTVRNVSWNGNLGAGQSTTYGFTGTGSAPSSATVSCS

SEQ ID NO: 55 - protein, 653 aa, Nonomuraea jiangxiensis
MRTNALPPPRTRRSFGGFRRAVAVGVLALAGTIAPLALSVPADAAETTLGAAAMQSNRYFGAAIAAGKLNESAYTTIANREFNMVTPENEMKIDATEPNRGQFTFTNADRIYNWAVQNGKRVRGHTLAWHSQQPGWMQSLSGSTLRQAMIDHINGVMAHYRGNIYAWDVVNEAFADGNSGGRRDSNLQRTGNDWIEVAFRTARAADPAAKLCYNDYNIENWTWAKTQGVYNMVRDFKSRGVPIDCVGLQAHFNSGSPYNSNFRTTLSSFAALGVDVQITELDIQGASATTYANVTNDCLAVPRCTGITVWGVRDSDSWRSGDTPLLFDGSGNKKPAYTSVLNALNSVNPNPDTTPPSTPGTPAASNVTSSGATLTWAASTDTGGSGLAGYNVYREQGTTDPQLGQSATNSITLTGLTAGTQYQVYVRARDGAGNLSGNSQLVTFTTQTGGGTDTTPPSTPGTPAASNVTASGATLTWTASTDTGGSGLAGYDVYREQGATDPLLGQSATNSITLTGLTAGTQYQVYVRARDGAGNLSGNSQPATFTTTGGGTGGSCTVTPTTQTQWPSGYVIDPVRVTAGTSAISGWTVTFTLPAGHTVTGSWNTQLTVSGQTVTARNAAHNGNLGPGASTAFGFQVSRPNGNTSLPSGYTCA

SEQ ID NO: 56 - protein, 634 aa, Kibdelosporangium aridum
MSRTVGRHAALLALLGTFLLPGTADAGTTLGASAAEKGRYFGAAVAAHKLGDSTYVGILNREFNMVTPENEMKIDATEPNQNQFSFGNADRIVNHAVSQGMRVRGHTLAWHSQQPGWMQNMSGSALRQAMLNHVTRVASYYRGKIYAWDVVNEAFADGSSGARRDSNLQRTGNDWIEAAFRAARAADPNAKLCYNDYNTDDWSHAKTQAVYRMVQDFKSRGVPIDCVGLQSHFNNNSPYPSNYRTTLSSFAALGVDVQITELDIEGAPPTTYGNVVRDCLAVARCNGITVWGIRDSDSWRASQTPLLFNNSGGKKPAYDAVLSALNSGSIPPPGGSNCSAGYVGLTFDDGPNSSTTPQLLNALRSAGVRATFFNVGQRVQQNPALTRSQIDAGMWVGNHSWTHPHLTQMTAAQITSELSQTQQALQQATGQTPRLFRPPYGETNSTVRQVQAQLGLTEVMWTVDSQDWNNASTAQIVQAASTLQPGGIILMHDGYQTTVNAIPQIVANLSSRNLCAGMISTNGQVVAPGTEPGNGSCTASYRTTQQWGDRFNGEVTIKAGSAAITSWTSTVTITAPQRVSTTWNGTASWDSSGTVMTMKPNGNGNLAAGASTTFGFTVMANGQWAQPSVSCGSP

SEQ ID NO: 57 - protein, 472 aa, Actinomadura madurae
MRANVIPKSRIRIRKRAILAGALGVLATAALVAPSPAAAAESTLGAAAAQNGRYFGTAIASGKLNDSVYTTIANREFNSVTAENEMKIDATEPQRGRFDFSAGDRVYNWAVQNGKQVRGHTLAWHSQQPGWMQSLEGGTLRQAMIDHINGVMAHYTGKIVQWDVVNEAFADGSSGARRDSNLQRTGNDWIEVAFRTARAADPAAKLCYNDYNIENWTWAKTQAVYSMVRDFKQRGVPIDCVGFQSHFNSGSPYNGNFRTTLQSFAALGVDVAITELDIQGASATTYANVTNDCLAVSRCLGITVWGVRDTDSWRSEQTPLLFDGNGNKKAAYTAVLNALNGGTTTPPDGAGTIKGDGSGRCLDVPNASTTDGTQVQLWDCHGGTNQQWTYTDASELRVYGDKCLDAAGTGNGARVQIYSCWGGDNQKWRLNSDGSVVGVQSGLCLDAAGTANGSAIQLYSCWNGGNQRWTRT

SEQ ID NO: 58 - protein, 484 aa, Nonomuraea indica
MRVDAMSPPTARRPLGARRRTLITGVLGALGLITALAPAIPADAAASTLGAAAAQSGRYFGTAIASGKLGDSAYTTIAGREFDMVTAENEMKIDATEPNRGQFTFTAGDRVYNWAVQNGKRVRGHTLAWHNQQPGWMQSLSGSSLRQAMINHINGVMGHYKGKIYAWDVVNEAYADGNSGGRRDSNLQRTGNDWIEVAFRTARAADPAAKLCYNDYNIDNWTWAKTQGVYNMVRDFRARGVPIDCVGLQSHFNANSPYNSNYRTTIQSFAALGVDVQITELDIQGGSATTYANVVRDCLAVPRCTGISVWGVRDSDSWLGAGATPLLFDGNGNKKAAYTAVLDVLNGGSTTPPPGDAGQIRNTASGRCVDVPNSATADGTAVQLWDCNGQSNQRWTRTAAGELKYGDKCLDAGGTGNGARIQIYSCWGGDNQKWRLNSDGTIVGVQSGLCLDAVGGGTGNGTGLQLYGCWGGGNQRWDYNPGTA

SEQ ID NO: 59 - protein, 680 aa, Saccharothrix syringae
MSRTAVTAAGRSEARGRSRRAAVVVGAIGLLSAAAVVLPNVATAGTTLGASAAESGRYFGTAVAANKLSDSTYVGILNREFDMVTAENEMKMDATEPNQNQFSFGNGDRIVNHARNQGKRVRGHALAWHSQQPGWMQNMSGTALRNAMLNHVTQVATYYKGKIYAWDVVNEAYADGSSGGRRDSNLQRTGNDWIEAAFRAARAADPNAKLCYNDYNTDNWSHAKTQGVYRMVQDFKSRGVPIDCVGFQAHFNSGNPVPSNYHTTLQNFADLGVDVQITELDIEGSGSTQAQQYQGVVQACLAVTRCTGITVWGIRDSDSWRSSGTPLLFDGSGNKKAAYTSVLNALNAGSTAPPSTTTTTTPPNTSDCRAGYVGLTYDDGPNGSTTTQLLNALRSAGLRATFFNQGNRVQQNPGLAKAQRDAGMWVGNHSWSHPHMTQLSQSQMASEISQTQQAIQSATGEAPKLFRPPYGETNSTLKSVEAQYGLTEVLWSVDSQDWNNASTAQIVQAASTLQNGGVILMHDGYQTTINAIPQIAANLASRGLCAGMISTSTGQAVAPNDNPPTTGPTTTTTTTSQQPGGSCTATYRTTQQWGDRFNGEVTVRAGASAITSWTATVTVTSPQKVSATWNGTPSWDSSGNVMTMKPNGNGNLAAGASTTFGFTVMTNGQWAAPTVSCRTP

SEQ ID NO: 60 - protein, 471 aa, Cellulomonas cellasea
MNRSGLRPPSSTHRRRTGAVLVAALAVTLAGATLPAQGAGSTLQAAAAESGRYYGTAIAANKLSDSTYTTIANREFNMITAENEMKMDATEPSQNQFNYSSGDRIVSWARSNGKRVRGHALAWHSQQPGWMQNMSGTALRNAMLNHVTQVATHYKGQIYAWDVVNEAYADGSSGARRDSNLQRTGNDWIEAAFRAARAADPAAKLCYNDYNTDNWSHAKTQGVYTMVRDFKSRGVPIDCVGFQAHFNSGNPVPSNYDVTLRNFAALGVDVQITELDIEGSGSSQAQQYAGVHQACLSVARCTGVTVWGVRDTDSWRASGTPLLFDGSGNKKAAYTSTLNALNAGGTTTPNPTPNPTTPQPTPTTTTPPVTGTGSCTATYSEGQKWGDRFNGVVTIRANSAITSWTSTVTVSQAQRITSTWSGTPSWDSSGKVMTMRPAGNGTLAAGQTTSFGFTVLHGGDWTWPRVTCSAS

SEQ ID NO: 61 - protein, 457 aa, Couchioplanes caeruleus
MHKPRTPLRALGVAAAAAVVAAGSVVALTSTAEAATTLGASAAATGRYFGTAVAANKLSDSTYVGILDREFNAVTPENEMKWDATEPNQNQFNYSSADRIVSHAQAQNMRIRGHALAWHQQQPGWAQNLSGTALRNAMLNHVTTVAAHYKGQIYAWDVVNEAFDDGGNGARRDSNLQRTGNDWIEAAFRAARAADPGAKLCYNDYNTDGQNAKSNAVYAMVQDFKSRGVPIDCVGFQSHFNAQSPVPSDYQANLQRFANLGVDVQITELDIEGSGQTQADNFGRVVKACLAVSRCTGITVWGIRDSDSWRASGTPLLFDSSGNKKAAYTSTLNALNSGGTTPPSDPPTSPSTPPSTPTDPGTGACTATISLNQWNGGFVATVRVSAGSSALSGWSVSTTLPSGAAVTNSWSSQSSGSSGTVRFANVSYNGSVAAGGSTEFGFQGTGTGPSSATCSAS

SEQ ID NO: 62 - protein, 473 aa, Glycomyces tritici
MITSTPRRRRRVWSTIAAVATASLAATTALVMLPGTAQAGTTLGASAAESGRYFGAAVSTQYLSESAYANTLGAEFNSVVAENAMKWDATEPNPGQFNFSGGDQLVNWAQARGMKVRGHTLVWHAQQPGWAQNLTGQNLRNAMLNHIAGVANHYEGDVFAWDVVNEAFEWDGSRRQSNLQRQLGNGWIEEAFRAADAADPTATLCYNDYGTDSINAKSTAIYNMVRDFKSRGVPIDCVGFQTHIAHNENLSSYQANLQRFADLGVDVEITELDVGQGSGQAATYGTVTNACMAVARCKGITVWGITDKYSWRDDNPLLFDGNYQKKQAYHTVLAALNNGNPGGGGGKHITSAASGRCLDVPNQSTTNGTQLQIYDCWTGANQQWTVANGEISVYSGGSKKCLDASGGGTANGTAAIIWSCHGGANQRWNVNSNGTITNAASGLCLDVAAAATANGSKVQLWSCTGGSNQRWTV

SEQ ID NO: 63 - protein, 475 aa, Actinoplanes auranticolor
MTVIAARARLLLGAACAVVIAVSSVAVATLAHADLTGPPGTTLKAAAERSGRYFGAAMGRDRLTDNGFLTIANREFDMMTAVNEMKPDATEPNRGQFDFRAGDAIYNWATQRGMRFRGHTLAWHAQQPRFWGSLSGSALRQAMIDHINGVMAHYKGKLYAWDVVNEAFAENGSRRSSNLQATGNDWIEAAFRAARAADPGVQLCYNDYNIENWTYAKTQGVYNMIRDFKARGVPIDCVGLQTHFTGGSSLPGNFQQTLSSFAALGVDVALTEADVTNASSSQYQGLTQACMNVPRCVGITTWGIRDSDSWRGNENPLLFDRNSNPKPAYTSVLNALNAASTTVPGTSTPPSTPPSTPPSTPPSTPPSTPPVTGQPGACTATYRTASTWNGGYQGEVTVANNGSAALTGWTVQLTLAGGQTVANVWNGINTGTSGTISVRNAAYNGSVGANASTSFGFLVNGSTGTAPGSLTCTSP

SEQ ID NO: 64 - protein, 456 aa, Sphaerisporangium rubeum
MVVIGTAVVVGAGVCAGAGAADAAGTLGAAAAGSGRDFGTAVQAGRLGEAAYVETLDREFTSVTPENEMKWDAVEPARGTFVFTAADRIVEHARARGMKIRGHTLVWHPGLPGWLTNLSSAEFRIAVNNHIATVMGHWRGQIDSWDVVNEAFQDGSGALRSTIFRSRLGDGYIEEAFRAARAADPAAKLCYNDYNIEDADAAKTRAVYAMVRDFKERGVPIDCVGVQSHFNASMPFPANYRRTIEQFAALGVDVQITQLDVDGGAAQAATYRAAVEACVAVPRCTGITVWGVPDHYSWRAPNTPLLFDRDYQKKPAYFAVLEALNAAGGEPTPAPTPTPSPTPTPTPTVTPVPVSCRVTAELWARSRTGYVIKPVTVRNTGRAAVSGWTVTFTLPQGHVVTGSWDAVLTVAGGTVTARNAGHNGTLAPGATATFGFQVRRASGGTALPSGYACRAG

SEQ ID NO: 65 - protein, 557 aa, Phytoactinopolyspora halotolerans
MITRHRRQAILASMALVGATLVVPIGAAQAQDTLRGAADAQGLRMGAAVANGPLSSDAQYRNILGTEFNSVTAENSMKWESLQPSQGQFTFSQGDAIVDFAQANNQAVYGHTLVWHNQTPSWVQNLSGQQLDDAMQTHITTVLDHYEGQVEAWDVANEVIDDGANLRNSFWLQGLGEGYIADAFRYADAADPNAKLYINDYNIDGINAKSNAYYDLVSGLLNQGVPIDGIGLQAHMILGQVPSSLEDNIRRFAQLGLEVRITELDIRMDLPVTQAKLEQQRQDYAAVVDACMSVDGCVGVTTWGFTDAHSWVPDQFGGQGAALPFDENYNKKPAYYGILDTLDGGTPNDTTPPTQPGAPQISDVTSNSASLTWSASSDSGGSGLAGYSVYREQSGNDQLLASPSTNSVTLTGLDPQTQYSVYVVARDGAGNTSSPSTAASFTTQEGPGGGGACDVEYTANNWGGSEGFTASVTITNTGSSQLNGWTLGFTFPGDQSVREGWSATWSQSGADVTAESIGWNDALAPGGSTTVGFNGTYSGSNPEPTEFTLNGEPCSVS

SEQ ID NO: 66 - protein, 466 aa, Glycomyces paridis
MHTRKRRRALIAGVVTVATAALGIGLSTQFASAQTTLRGAADEAGIDIGIAVDANQLQNNATYRNLVATEFNSLTAENAMKWDATEPSDNSWNFSGADAIVDFAEDNDQTVHGHTFVWHSQTPQWVQNLSASAMQAAMVDHINTLANRYEGRVDSWDVVNEVVSDNNGAMRDSFWRNTLGDGYITTAFQTARAADPNADLYINDYSIEGDNAKSDRIYTIAQGLNSQNLIDGVGFQSHLILGQVPSTMEANLQRFIDLGLKVRITELDIRIQTPADANELQQQANDYERVVSICAELADCSGVTVWGLRDADSWVPGVFPGYGAPLLFDDNFGKKPAYNAVLEALGGDGSEEPTTTDPGTTTPPPTGDGDCTAEIDVVNDWGSGWQGNVLITADNGAVNGWTLTWTWPSGQSITSSWNATVTSSGSSVTASDVGWNANIAAGQTMNAWGFVGSGSSAAPQVTCTAD

SEQ ID NO: 67 - protein, 486 aa, Streptomonospora alba
MRLVRHGGHETPRRTRAGSARRVAGAALATFLGAAVLAPAGPAAADSPLRDHAAQQGFEIGAALGVDHLQNDSQFADLAATEFNAATAENSMKWESTEPSRGQFDFSGADAFMDFAQQNNQKVRGHTLVWHSQLPSWVENGNFSQSELRSVMEDHIDEVAGRYAGEVAYWDVANEIFEGDGSWRNSVFYDTLGPDFVADALRMTAEADPNAELWLNDYSIDGINAKSDAYYNLIQDLQAQGVPIDGIGLQAHLINGQVPGDLQQNIQRFADLGIKVAITELDVRIDMPASQSELQQQAQDYRAVMDACLAVNGCVGVTVWGIVDKYSWVPDTFEGEGAPLLFNDDYQPKPAYDAVHEALGGSSDDDDDDDNGGGEDPPPTGPCEVSYSVANEWNSGFTGQVTVTNGGSSALNGWDLQFDFSGGQQITNGWNADWSQSGSTVTASNTEWNGSVPAGGSVDIGFNASHSGSNPEPGSFALNGESCSVS

SEQ ID NO: 68 - DNA, 1413 bp, Synthetic construct
  source 1..1413: mol_type = other DNA
atgacgccaagttcaatgtcccgtcgggcccgcgttgcgtcagctcttgccatcgtgacgttgggcgctactatcgctactacgattcctgcccaagccgccggctctacattacaagcggctgcttctgaaagtggtcgttatttcggtacagcgattgctgcgtttaaattgaatgattcaacgtacagctccattgcgaatcgggagttcaatatgattacagcagaaaatgaaatgaaaatggacgcgactgagccgtctcagaataatttttcgtattcgagtggcgatcagattcttaattgggcacggtctaatggtaagcgtgttcgtggccatgcattggcctggcattcgcagcaaccggggtggatgcaaaatatgtccggtacacagttacgcaatgccatgttgaatcatgtgacgcaggttgctacgcattataaaggcaagatttatgcatgggatgtcgttaatgaagcctatgcggactctggtggggggcgtcgtgacagtaatcttcaacggaccggtgatgattggattgaagctgcttttcgcgctgcgcgggcggcagatccaggtgccaaactttgctataatgattataatacagacaattggacgtgggctaaaacacagggtgtctacaacatggttaaagattttaaagctcgtggggtgccgattgattgcgttggctttcaatctcatttcaattccgggtcgccgtatccgtcaaattatcgtacaaccttacaaaatttcgccgccctgggtgtggaagttcagattaccgaattagatatcgaaggttcggggcaacaacaggcacagacttatgccaatgtcgtcgctgattgcttggcagtgaaggcttgcaccggtatcacagtttggggcgtcagagactcagactcttggcgcagctccgggacgccgttgttgtttgatggcagcggtaataaaaaggctgcgtatacctctaccttggacgctcttaaccggggtggagtcccgactgatccgaccactccgccgacagaccccacgacaccacccactgatcccacgacaccgcctaccgatcctaccactccgcctacggatcctacaaccccacccacggatccaactggccgttgcaccgcgagccttgcgattgccaacgcctggccgggtggatatcaggccacggtcacggtcaaagcagggtccagtagtatcaatggatggcgcgttacattaccttctggcgtttccactagtaacctgtggaatggcgtcctggcgaacggagtggtcacgaatgccccttataatggctctgttggcgcggggcaatccacgacctttggctttgtcggcaatggctcggcgcccagtgctggtagcgtgacttgtgcctga